(19) United States
(12) Patent Application Publication
(10) Pub. No.: US 2023/0194554 A1
(43) Pub. Date: Jun. 22, 2023

(54) USE OF BIOMARKER IN PREPARATION OF LUNG CANCER DETECTION REAGENT AND RELATED METHOD

(71) Applicant: Sun Yat-sen University Cancer Center (The Affiliated Cancer Hospital of Sun Yat-sen University, The, Guangzhou (CN)

(72) Inventors: Yumin HU, Guangzhou (CN); Yao YAO, Guangzhou (CN)

(21) Appl. No.: 18/073,905

(22) Filed: Dec. 2, 2022

Related U.S. Application Data
(63) Continuation-in-part of application No. PCT/CN2021/122812, filed on Oct. 9, 2021.

(30) Foreign Application Priority Data
Oct. 10, 2020 (CN) 202011077018.3

(51) Int. Cl. G01N 33/68

(52) U.S. Cl. CPC G01N 33/6893

(57) ABSTRACT

A medical diagnosis screens a biomarker for lung cancer detection by utilizing serum metabonomics. The medical diagnosis includes a biomarker for differential diagnosis between patients with lung cancer and healthy people, and between patients with lung cancer and patients with benign pulmonary nodules, and a biomarker for differential diagnosis between patients with lung cancer and healthy people, and between patients with lung cancer and patients with benign pulmonary nodules according to gender differences between a man and a woman. The biomarker is of great significance especially in the differential diagnosis of whether a patient with nodules in the lung has lung cancer.

### CROSS-REFERENCE TO RELATED APPLICATIONS

The present application claims the priority of the prior Chinese application No. 2020110770183 filed on Oct. 10, 2020, all contents of which are incorporated as a part of the present application, and which is a partial continuation of PCT application No. PCT/CN2021/122812 filed on Oct. 9, 2021.

### TECHNICAL FIELD

The invention relates to the field of medical diagnosis, and in particular to the diagnosis of lung cancer by screening a biomarker by utilizing serum metabonomics, especially the differential diagnosis between benign pulmonary nodules and lung cancer.

### BACKGROUND OF THE INVENTION

Lung cancer is one of the most common malignant tumors in the world, and one of the cancers with the highest mortality. According to the latest data released by China in 2018, there are 2.1 million new cases of lung cancer in China, ranking first among malignant tumors, accounting for 18.4% (ranking first) of all tumor deaths, and the number of deaths is 1.8 million (ranking first), accounting for more than a quarter of malignant tumor deaths. Early diagnosis is of great significance to improve the treatment prognosis and survival rate of tumor patients. Currently, the confirmed diagnosis of lung cancer mainly depends on pathological examination of tissues or cells obtained through invasive paracentesis and bronchoscopy. CT imageological examination is a main auxiliary diagnostic means, but there are still some challenges in the differential diagnosis of benign or malignant pulmonary nodules. Serological examination of lung cancer, such as a carcinoembryonic antigen, a keratin fragment, a squamous cell carcinoma antigen, etc., can be used as an auxiliary diagnosis or follow-up monitoring of lung cancer, but its current sensitivity and specificity still need to be improved.

In recent years, with the rapid development of mass spectrometry, the study on application of Metabolomics in disease diagnosis has gradually attracted widespread attention. Metabonomics is a new discipline to qualitatively and quantitatively analyze a small-molecule metabolite with a relative molecular weight less than 1,000 in an organism. Metabolome refers to all of the low-molecular-weight metabolites of a certain organism or cell in a specific physiological period, and many life activities in the cell take place at the level of metabolites. Therefore, the detection and identification of metabolome can judge the pathophysiological state of the organism, and it is possible to find out a marker related to its pathogenesis. Therefore, the metabonomics has a wide application prospect in the field of clinical medicine. Metabolites in serum are stable and quantifiable, which provides a possibility of non-invasive diagnosis for clinical application.

Currently, there are no metabolic markers that can be used for diagnosis of lung cancer or for differential diagnosis between lung tumors and benign nodules. However, it is of great clinical significance to differentially diagnose whether a patient with lung nodules is a patient with lung cancer.

Meanwhile, it is worth paying attention to that male and female patients have their own characteristics in the pathogenesis, etiology, diagnosis, pathology, molecular biology, treatment and prognosis of some tumors (including lung cancer), while the prior art does not distinguish the serum metabolic markers of tumors (including lung cancer) according to gender.

It is necessary to improve the traditional technology, hoping to have a method and reagent for diagnosing or predicting lung cancer.

### SUMMARY OF THE INVENTION

In the invention, serum samples of healthy people, a patient with benign pulmonary nodules and a patient with lung cancer (lung malignant tumor) are collected, and subjected to metabonomics analysis and typing of profiling by utilizing liquid chromatography-high resolution mass spectrometry (LC-HRMS), so as to screen out a biomarker among healthy people, the patient with benign pulmonary nodules and the patient with lung cancer, and the biomarker is further distinguished according to gender to find out a biomarker among healthy people, the patient with benign pulmonary nodules and the patient with lung cancer of the same gender.

An objective of the present invention is to find a metabolic biomarker between healthy people and a patient with lung cancer, and between a patient with benign pulmonary nodules and the patient with lung cancer, so as to be used for the diagnosis of lung cancer, especially for the early differential diagnosis of whether a patient with nodules has lung cancer. Moreover, considering the effect of gender differences, the invention differentiates according to gender, looking for a biomarker for diagnosis of lung cancer in a man or woman.

In an aspect, the invention provides a method for screening a lung cancer biomarker based on serum metabolomics, comprising the specific steps of:

(1) collecting serum samples of a patient with lung cancer, a patient with benign pulmonary nodules and healthy people;

(2) extracting serum metabolites;

(3) conducting detection of the extracted serum metabolites by liquid chromatography-mass spectrometry (LC-MS) and preprocessing the data;

(4) grouping the samples by partial least squares discriminant analysis, so as to screen out differential metabolites or differential biomarkers in different groups in connection with significance analysis; and

(5) mining biomarkers of lung cancer and applications thereof according to the screened differential metabolites, for example how to use these markers to diagnose or predict a patient with lung cancer, or to differentially diagnose a patient with lung cancer from healthy people or a patient with nodules.

In some embodiments, the specific implementation of the step (1) is: the serum samples are derived from patients with lung cancer, patients with benign pulmonary nodules and healthy people of different genders and age groups. Here, the so-called population with lung cancer, population with benign pulmonary nodules and healthy population have been diagnosed and confirmed, such as the population with lung cancer, population with (benign) pulmonary nodules or healthy population (without nodules) confirmed by histology or later symptoms.

In some embodiments, the specific implementation of the step (2) is: the serum metabolites are extracted by employing a three-phase extraction method of methyl tert-butyl ether:methanol:water (10:3:2.5, v/v/v), wherein methanol and methyl tert-butyl ether are sequentially added into 50 μL of serum, the mixture is incubated with shaking on ice for 1 hour, then added with water, subjected to vortex shaking, and subsequently centrifuged, the subnatant is taken and spin-dried in a low-temperature vacuum dryer, and the obtained dry extract of the serum metabolites is stored in a refrigerator at −80° C.

Considering the batch effect of sample pretreatment, in this study, processing of each batch of experimental samples is conducted simultaneously with the processing of one Reference serum for subsequent data correction.

The specific implementation of the step (3) is: the dry extract of the serum metabolites is reconstituted and centrifuged, then the supernatant is taken to make samples to be tested, and all of samples are detected by liquid chromatography-high resolution mass spectrometry (LC-HRMS). The m/z ion, retention time and peak area are extracted from the original data, subjected to data normalization, and finally searched in a database for identification, so as to obtain a data matrix for subsequent analysis.

Further, the specific implementation of the step (4) is: data filtering is performed on the data matrix of liquid chromatography-high resolution mass spectrometry, and the remaining data is subjected to partial least squares discriminant analysis for sample grouping, and thus three groups, a lung cancer group, a benign pulmonary nodules group and a healthy group, can be obviously clustered.

In some embodiments, the specific implementation of the step (5) is: a compound with an FDR value less than 0.05 and meanwhile with a VIP greater than 1 is screened out as a differential metabolite, and the fold change is calculated. Furthermore, combined with biological significance, the differential metabolic markers of the patient with lung cancer, the patient with benign pulmonary nodules and healthy people are mined, and metabolic pathways are analyzed.

In some embodiments, the differential metabolic markers for the patient with lung cancer, the patient with benign pulmonary nodules and healthy people of the same gender are screened according to steps (4) and (5) according to gender.

In a second aspect of the present invention, provided is a method for detecting whether an individual has lung cancer, which includes the steps of: detecting a marker in a blood or serum sample of the individual, wherein the biomarker is selected from one or more of: 1-Methylnicotinamide, 2-Ketobutyric acid, 2-Octenoylcarnitine, 2−Pyrrolidone, 2-trans,4-cis-Decadienoylcarnitine, 3b,16a-Dihydroxyandrostenone sulfate, 3-Chlorotyrosine, 3-hydroxybutyryl carnitine, 3-hydroxydecanoyl carnitine, 3-hydroxydodecanoyl carnitine, 3-hydroxyoctanoyl carnitine, 4-oxo-Retinoic acid, 7-Methylguanine, Acetophenone, Acetylcarnitine, Alanine, alpha-Eleostearic acid, Aminoadipic acid, Arabinosylhypoxanthine, Asparagine, Bilirubin, Carnitine, Choline Sulfate, cis-5-Tetradecenoylcarnitine, Citrulline, Creatinine, Cyclohexaneacetic acid, Diethylamine, Dihydrothymine, Dihydroxybenzoic acid, Docosahexaenoic acid, Ecgonine, Ergothioneine, Ethyl 3-oxohexanoate, Glutamine, Hexanoylcarnitine, Hippuric acid, Homo-L-arginine, Hydroxybutyric acid, Hypoxanthine, Inosine, Isoleucine, Kynurenine, Lactic acid, Leucine, Linoleyl carnitine, Lysine, Methylacetoacetic acid, N6,N6,N6-Trimethylysine, N-Acetyl-L-alanine, Nicotine, Octanoylcarnitine, 5-Oxoproline, Phenylalanine, Pilocarpine, Propionylcarnitine, Pyruvic acid, Serotonin, Succinic acid semialdehyde, Trimethylamine N-oxide, Tyrosine, Uridine, Urocanic acid, Xanthine, 4-Hydroxyphenylacetic acid, Dehydroepiandrosterone sulfate, Androsterone sulfate, Dihydrotestosterone sulfate, Epiandrosterone sulfate, Citric acid, Uric acid, Pantothenic acid, Indole-3-acetic acid, gamma-Butyrobetaine, Calcitriol, all-trans-retinal, 3,4-dihydroxyphenylacetic acid, Caprylic acid, Arachidic acid, Hydrocortisone Valerate, Dopamine, Tryptophan, 3-Hydroxybutyric acid, Arachidonic acid.

In some embodiments, the biomarker for diagnosing lung cancer is one of or a combination of several ones of: alpha-Eleostearic acid, 2-Ketobutyric acid, 2-Octenoylcarnitine, 2-trans,4-cis-Decadienoylcarnitine, 3-Chlorotyrosine, 3-hydroxydecanoyl carnitine, 3-hydroxydodecanoyl carnitine, 3-hydroxyoctanoyl carnitine, Acetophenone, Arabinosylhypoxanthine, Cyclohexaneacetic acid, Dihydroxybenzoic acid, Docosahexaenoic acid, Ecgonine, Ethyl 3-oxohexanoate, Hexanoylcarnitine, Hippuric acid, Homo-L-arginine, Hypoxanthine, Lactic acid, N-Acetyl-L-alanine, Octanoylcarnitine, 5-Oxoproline, Pyruvic acid, Serotonin, Succinic acid semialdehyde, Xanthine. The aforementioned biomarkers have significant differences both between the patient with lung cancer and healthy people, and between the patient with lung cancer and the patient with benign pulmonary nodules, indicating that they are closely related to lung cancer and are not affected by whether there are benign pulmonary nodules. Therefore, they can be used for differential diagnosis of lung cancer and benign pulmonary nodules, and can also be used for differential diagnosis of the patient with lung cancer and healthy people (without nodules). In some embodiments, when 2-Ketobutyric acid, Hypoxanthine, Lactic acid, N-Acetyl-L-alanine, 5-Oxoproline, Pyruvic acid, Xanthine, Succinic acid semialdehyde among the aforementioned markers in the serum of an individual (including those with and without pulmonary nodules) are increased, it indicates that the individual has high possibility of suffering from lung cancer. In some embodiments, similarly, if other biomarkers are decreased at the same time, it further indicates a large possibility of suffering from lung cancer.

In some embodiments, during a process of comparing differential metabolites between the patient with lung cancer and healthy people, between the patient with lung cancer and the patient with benign pulmonary nodules, between the patient with lung cancer and healthy people in men and women, and between the patient with lung cancer and the patient with benign pulmonary nodules in men and women, it is found that: 3-hydroxydecanoyl carnitine, 3-hydroxyoctanoyl carnitine, Arabinosylhypoxanthine, Cyclohexaneacetic acid, Ecgonine, Ethyl 3-oxohexanoate, Hippuric acid, Homo-L-arginine, Hypoxanthine, Octanoylcarnitine, 5-Oxoproline have significant differences between the patient with lung cancer and healthy people or the patient with benign pulmonary nodules (including if they are distinguished according to gender), which indicates that these differential metabolites are more closely related to lung cancer, and are not affected by benign pulmonary nodules and gender. Therefore, they can be used for differential diagnosis between the patient with lung cancer and healthy people and between the patient with lung cancer and the patient with benign pulmonary nodules, and can also be used for differential diagnosis between the patient with lung cancer and healthy people and between the patient with lung cancer and the patient with benign pulmonary nodules in men or women.

When in the serum of an individual (including men and women, with and without nodules), Hypoxanthine and 5-Oxoproline are increased, while 3-hydroxydecanoyl carnitine, 3-hydroxyoctanoyl carnitine, Arabinosylhypoxanthine, Cyclohexaneacetic acid, Ecgonine, Ethyl 3-oxohexanoate, Hippuric acid, Homo-L-arginine, and Octanoylcarnitine are decreased, it indicates that the individual has high possibility of suffering from lung cancer. In some embodiments, similarly, if other biomarkers are decreased at the same time, it further indicates a large possibility of suffering from lung cancer.

In some embodiments, the biomarker is selected from one or more of the following Table 2 when used for diagnosing whether an individual without pulmonary nodules has lung cancer. The increase of one or more of Hypoxanthine, Lactic acid, Xanthine, N-Acetyl-L-alanine, Succinic acid semialdehyde, Pyruvic acid, 2-Ketobutyric acid, Methylacetoacetic acid, and 5-Oxoproline, or the decrease of other markers, indicates a large possibility of suffering from lung cancer. In some embodiments, similarly, if other biomarkers are decreased at the same time, it further indicates a large possibility of suffering from lung cancer.

In some embodiments, when it is clinically known that there is a mass or nodules in the lung of a patient, the biomarkers are selected from one or more of those in Table 3 when used for differential diagnosis between lung cancer and benign lung nodules. In some embodiments, the increase of one or more of the following markers: Hypoxanthine, Lactic acid, Xanthine, Dihydrothymine, N-Acetyl-L-alanine, 5-Oxoproline, 2−Pyrrolidone, Hydroxybutyric acid, Succinic acid semialdehyde, Pyruvic acid, 2-Ketobutyric acid, or alternatively the decrease of other markers, indicates a high possibility of suffering from lung cancer.

In some embodiments, the biomarker used for differential diagnosis between lung cancer and benign pulmonary nodules is one of or a combination of several ones of: 1-Methylnicotinamide, 2−Pyrrolidone, 4-oxo-Retinoic acid, 7-Methylguanine, Acetylcarnitine, Bilirubin, Choline Sulfate, cis-5-Tetradecenoylcarnitine, Citrulline, Creatinine, Diethylamine, Dihydrothymine, Glutamine, Hydroxybutyric acid, Inosine, Kynurenine, Linoleyl carnitine, Lysine, Trimethylamine N-oxide. These biomarkers have significant differences between the patient with lung cancer and the patient with benign pulmonary nodules, but there is no significant difference between the patient with lung cancer and healthy people, which indicates that these biomarkers are preferred and specific biomarkers for distinguishing the patient with lung cancer from the patient with benign pulmonary nodules, and cannot distinguish the patient with lung cancer from healthy population (without nodules). These biomarkers have more practical significance. The possibility of further detection of canceration is only possible when nodules are generally found in the process of physical examination or diagnosis. At this time, apart from routine puncture biopsy, an effective way of preliminary screening is to conduct preliminary screening by detecting whether there are changes or abnormalities, such as significant changes, in one or more of the aforementioned markers in a blood sample.

In some embodiments, the method for differential diagnosis of lung cancer and benign pulmonary nodules through a blood sample includes: detecting a biomarker in a blood sample, wherein the biomarker is selected from one of or a combination of several ones of: 1-Methylnicotinamide, 2-Octenoylcarnitine, 3-hydroxydecanoyl carnitine, 3-hydroxyoctanoyl carnitine, 4-oxo-Retinoic acid, 7-Methylguanine, Arabinosylhypoxanthine, Cyclohexaneacetic acid, Ecgonine, Ethyl 3-oxohexanoate, Hippuric acid, Homo-L-arginine, Hypoxanthine, Inosine, Lactic acid, Octanoylcarnitine, 5-Oxoproline, Trimethylamine N-oxide. These biomarkers have significant differences between the patient with lung cancer and the patient with benign pulmonary nodules (including men and women), and have significant differences between the patient with lung cancer and the patient with benign pulmonary nodules in men or women, which indicates that these biomarkers are not affected by gender and can effectively distinguish lung cancer from benign pulmonary nodules.

In some embodiments, the biomarker is selected from one or more of those in Table 4 when used for determining whether a man without pulmonary nodules has lung cancer. The increase of one or more of Hypoxanthine, N-Acetyl-L-alanine, Pyruvic acid, and 5-Oxoproline, or the decrease of one or more of other biomarkers, indicates that the man has a high possibility of suffering from lung cancer.

In some embodiments, when it is clinically known that there is a mass or nodules in the lung of the male patient, the biomarker is selected from one or more of those in Table 5 when used for determine whether the mass or nodules is lung cancer or benign pulmonary nodules. The increase of one or more of Hypoxanthine, N-Acetyl-L-alanine, Pyruvic acid, 5-Oxoproline, Lactic acid, Dihydrothymine, Aminoadipic acid, and N6,N6,N6-Trimethylysine, or the decrease of one or more of other markers, indicates that the man has a large possibility of suffering from lung cancer.

In some embodiments, the biomarker used for determining lung cancer and benign pulmonary nodules in men is one of or a combination of several ones of: 1-Methylnicotinamide, 2-trans,4-cis-Decadienoylcarnitine, 3-hydroxydecanoyl carnitine, 3-hydroxydodecanoyl carnitine, 3-hydroxyoctanoyl carnitine, 4-oxo-Retinoic acid, 7-Methylguanine, Acetylcarnitine, alpha-Eleostearic acid, Arabinosylhypoxanthine, Cyclohexaneacetic acid, Diethylamine, Docosahexaenoic acid, Ecgonine, Ethyl 3-oxohexanoate, Glutamine, Hippuric acid, Homo-L-arginine, Hypoxanthine, Inosine, Linoleyl carnitine, N-Acetyl-L-alanine, Octanoylcarnitine, 5-Oxoproline, Pyruvic acid, and Trimethylamine N-oxide. These biomarkers have significant differences both between the patient with lung cancer and the patient with benign pulmonary nodules in men and between the patient with lung cancer and healthy people in men, which indicates that these biomarkers are closely related to lung cancer in men, and they can be used for not only distinguishing the patient with lung cancer from the patient with benign pulmonary nodules in men, but also distinguishing the patient with lung cancer from healthy people (without nodules) in men.

In some embodiments, the biomarker used for determining lung cancer and benign pulmonary nodules in men is one of or a combination of several ones of: 2-Octenoylcarnitine, 3-hydroxybutyryl carnitine, Aminoadipic acid, Bilirubin, Dihydrothymine, Ergothioneine, Lactic acid, N6,N6,N6-Trimethylysine, Nicotine. These biomarkers have significant differences both between the patient with lung cancer and the patient with benign pulmonary nodules in men and between the patient with lung cancer and healthy people in men, which indicates that these biomarkers can be used for distinguishing the patient with lung cancer from the patient with benign pulmonary nodules in men, but cannot be used for distinguishing the patient with lung cancer from healthy people (without nodules) in men.

In some embodiments, the biomarker used for determining lung cancer and benign pulmonary nodules in men is one of or a combination of several ones of: alpha-Eleostearic acid, 2-trans,4-cis-Decadienoylcarnitine, 3-hydroxydodecanoyl carnitine, Acetylcarnitine, Bilirubin, Diethylamine, Dihydrothymine, Docosahexaenoic acid, Glutamine, Linoleyl carnitine, N-Acetyl-L-alanine, Pyruvic acid, 3-hydroxybutyryl carnitine, Aminoadipic acid, Ergothioneine, N6,N6,N6-Trimethylysine, Nicotine. These biomarkers have significant differences between the patient with lung cancer and the patient with benign pulmonary nodules in men, but have no significant difference the patient with lung cancer and the patient with benign pulmonary nodules in women, which indicates that these biomarkers are related to gender and can be used for distinguishing lung cancer from benign pulmonary nodules in men, but not in women.

In some embodiments, the biomarker used for determining lung cancer and benign pulmonary nodules in men is one of or a combination of several ones of: 3-hydroxybutyryl carnitine, Aminoadipic acid, Ergothioneine, Nicotine. These biomarkers only have significant differences between the patient with lung cancer and the patient with benign pulmonary nodules in men, but have no significant differences between the patient with lung cancer and healthy people (including men and women), between the patient with lung cancer and the patient with pulmonary nodules (including men and women), between the patient with lung cancer and healthy people in men, between the patient with lung cancer and healthy people in women, and between the patient with lung cancer and the patient with pulmonary nodules in women, which indicates that these compounds are specific biomarkers for lung cancer and benign pulmonary nodules in men, and can only be used for distinguishing lung cancer from pulmonary nodules in men, but not for distinguishing lung cancer from pulmonary nodules or distinguishing lung cancer from healthy people (without nodules) in women.

In some embodiments, the biomarker is selected from one or more of those in Table 6 when used for determining whether a woman with pulmonary nodules has lung cancer. The increase of one or more of Alanine, Linoleyl carnitine, Pyruvic acid, Methylacetoacetic acid, Hypoxanthine, Lactic acid, Xanthine, 2−Pyrrolidone, Succinic acid semialdehyde, 2-Ketobutyric acid, and 5-Oxoproline, or the decrease of one or more of other markers, indicates that the woman has a high possibility of suffering from lung cancer.

In some embodiments, the biomarker used for determining lung cancer and benign pulmonary nodules in women is one of or a combination of several ones of: 1-Methylnicotinamide, 2-Ketobutyric acid, 2−Pyrrolidone, 3-Chlorotyrosine, 3-hydroxydecanoyl carnitine, 3-hydroxyoctanoyl carnitine, 4-oxo-Retinoic acid, 7-Methylguanine, Acetophenone, Arabinosylhypoxanthine, Choline Sulfate, Citrulline, Creatinine, Cyclohexaneacetic acid, Ecgonine, Ethyl 3-oxohexanoate, Hexanoylcarnitine, Hippuric acid, Homo-L-arginine, Hypoxanthine, Inosine, Lactic acid, Lysine, Octanoylcarnitine, 5-Oxoproline, Serotonin, Succinic acid semialdehyde, Trimethylamine N-oxide, Xanthine. These biomarkers have significant differences both between the patient with lung cancer and the patient with benign pulmonary nodules in women and between the patient with lung cancer and healthy people in women, which indicates that these biomarkers are closely related to lung cancer in women, and they can be used for not only distinguishing the patient with lung cancer from the patient with benign pulmonary nodules in women, but also distinguishing the patient with lung cancer from healthy people (without nodules) in women.

In some embodiments, the biomarker used for determining lung cancer and benign pulmonary nodules in women is one of or a combination of several ones of: 2-Octenoylcarnitine, cis-5-Tetradecenoylcarnitine, Kynurenine, Phenylalanine. These biomarkers have significant differences both between the patient with lung cancer and the patient with benign pulmonary nodules in women and between the patient with lung cancer and healthy people in women, which indicates that these biomarkers can be used for distinguishing the patient with lung cancer from the patient with benign pulmonary nodules in women, but cannot be used for distinguishing the patient with lung cancer from healthy people (without nodules) in women.

In some embodiments, the biomarker used for determining lung cancer and benign pulmonary nodules in women is one of or a combination of several ones of: 2-Ketobutyric acid, 2−Pyrrolidone, 3-Chlorotyrosine, Acetophenone, Choline Sulfate, cis-5-Tetradecenoylcarnitine, Citrulline, Creatinine, Hexanoylcarnitine, Kynurenine, Lysine, Serotonin, Succinic acid semialdehyde, Xanthine, Phenylalanine. These biomarkers have significant differences between the patient with lung cancer and the patient with benign pulmonary nodules in women, but have no significant difference the patient with lung cancer and the patient with benign pulmonary nodules in men, which indicates that these biomarkers are related to gender and can be used for distinguishing lung cancer from benign pulmonary nodules in women, but not in men.

In some embodiments, the biomarker used for determining lung cancer and benign pulmonary nodules in women is Phenylalanine. This biomarker only has significant differences between the patient with lung cancer and the patient with benign pulmonary nodules in women, but have no significant differences between the patient with lung cancer and healthy people (including men and women), between the patient with lung cancer and the patient with pulmonary nodules (including men and women), between the patient with lung cancer and healthy people in women, between the patient with lung cancer and healthy people in men, and between the patient with lung cancer and the patient with pulmonary nodules in men, which indicates that this compound is a specific biomarker for lung cancer and benign pulmonary nodules in women, and can only be used for distinguishing lung cancer from pulmonary nodules in women, but not for distinguishing lung cancer from benign pulmonary nodules or distinguishing lung cancer from healthy people (without nodules) in men.

In a third aspect of the invention, a model for identifying lung cancer and benign pulmonary nodules (including men and women) by a combination of various differential metabolites is established. The model parameters are the optimum model parameters. The model has an AUC of 0.955, sensitivity and specificity of 0.913 and 0.876 respectively as obtained by ROC analysis, which indicates that the model has high diagnostic accuracy.

In some embodiments, these models can be input into a computer system in advance, and when a biomarker is obtained, a diagnosis result is obtained through automatic calculation by the computer system. Therefore, the invention can provide a diagnosis system for lung cancer, which includes an operation module, wherein the operation or calculation module includes the following model equations. In some embodiments, the computer system further includes an output module for outputting the calculation result. In some embodiments, the computer system further includes an input module for inputting one or more detection results of the aforementioned biomarkers, and such detection results can be quantitative detection results or qualitative results. The established model here is not a finite model recited in the invention, as long as the biomarkers within the scope of the invention are applied to establish a model for lung cancer diagnosis, the model is within the scope of the invention. In some embodiments, the computer system further includes a negative control or reference data module.

In some embodiments, The model equation can be: Logit(P)=In[P/((1−P)]=5.553×VO4+2.92×V05+2.713×V06−0.332×V07−1.798×V10−7.922×V13−0.593×V14+0.643×V17−2.187×V19−0.992×V20−2.352×V33−1.441×V38+7.214×V39−1.22×V40−1.235×V42+1.61, wherein V04, V05, V06, V07, V10, V13, V14, V17, V19, V20, V33, V38, V39, V40, V42 are respectively the 5-Oxoproline, N-Acetyl-L-alanine, Hypoxanthine, Cyclohexaneacetic acid, Ethyl 3-oxohexanoate, Arabinosylhypoxanthine, Docosahexaenoic acid, Hydroxybutyric acid, Serotonin, Ecgonine, Lysine, Kynurenine, Inosine, 4-oxo-Retinoic acid, Linoleylcarnitine. In some embodiments, a cut-off value of P is 0.424, and when P>0.424, the possibility of being diagnosed with or suffering from lung cancer is very high.

In some embodiments, a model for identifying benign pulmonary nodules and lung cancer in men by a combination of multiple differential metabolites is established. As obtained by ROC analysis, the model has an AUC of 0.968, and sensitivity and specificity of 0.870 and 0.988 respectively, indicating that the model had high diagnostic accuracy. The model equation of the model is: Logit(P)=In[P/((1−P)]=6.283×MV02−0.646×MV10−2.758×MV13+1.864×MV15−1.126×MV19−1.145×MV27−3.918×MV30+1.494, wherein MV02, MV10, MV13, MV15, MV19, MV27, MV30 are respectively the 5-Oxoproline, Nicotine, Ecgonine, N6,N6,N6-Trimethylysine, Arabinosylhypoxanthine, Docosahexaenoic acid, Linoleyl carnitine. In some embodiments, a cut-off value of P is 0.701, and when P>0.701, the possibility of being diagnosed with or suffering from lung cancer is very high.

Moreover, a model for identifying benign pulmonary nodules and lung cancer in women by a combination of multiple differential metabolites is established. The model parameters are the optimum model parameters. As obtained by ROC analysis, the model has an AUC of 0.969, and sensitivity and specificity of 0.870 and 0.953 respectively, indicating that the model had high diagnostic accuracy. The model equation of the model is: Logit(P)=In[P/((1−P)]=10.742×FV05−1.031×FV08−7.442×FV09+11.839×FV13−2.617×FV15−3.030×FV20−1.413×FV23−2.278×FV29−6.905, wherein FV05, FV08, FV09, FV13, FV15, FV20, FV23, FV29 are respectively the 5-Oxoproline, Cyclohexaneacetic acid, Lysine, Phenylalanine, Serotonin, Kynurenine, Arabinosylhypoxanthine, 3-hydroxydecanoyl carnitine. In some embodiments, a cut-off value of P is 0.629, and when P>0.629, the possibility of being diagnosed with or suffering from lung cancer is very high.

In some embodiments, an ROC curve is established for each metabolic compound, and those compounds with large areas under the curve can be found, so that a batch of compounds can be chosen to establish a diagnostic model or obtain more reliable diagnostic results. It is generally understood that the reliability of the established model may be higher when more biomarkers are selected. For example, the sensitivity is higher when the accuracy is higher and the specificity is stronger. However, a single compound or several important compounds can also be selected for diagnosis, or for preliminary screening and detection. This detection method can be of a variety, for example, by utilizing the liquid-phase mass spectrometry combined detection of the invention, or by detecting one or more biomarkers of the invention at one time in a high-throughput manner, and of course, the detection of a small amount of several biomarkers is not excluded. Of course, an immune method can also be adopted to detect a small amount of several important compounds, such as the joint detection of 1, 2, 3, 4 or 5 biomarkers, which can also explain some problems.

Therefore, in some schemes, the biomarker used for determining whether a patient with pulmonary nodules (including men and women) has lung cancer is one of or a combination of two or three of 5-Oxoproline, Arabinosylhypoxanthine and Inosine. A model for identifying benign pulmonary nodules and lung cancer by a single differential metabolite is established, and it is found by establishing an ROC curve of each differential metabolite that, the AUCs (areas under curve) of 5-Oxoproline, Arabinosylhypoxanthine and Inosine are 0.736, 0.784 and 0.747, respectively, which are higher than those of other differential metabolites, indicating that the three differential metabolites have higher differential diagnosis values.

Moreover, when the model for identifying benign pulmonary nodules and lung cancer by a combination of various differential metabolites is established, it is found that 5-Oxoproline, Arabinosylhypoxanthine, and Inosine have the largest absolute values of model coefficients in the model, and the ORs (odds ratios) of 5-Oxoproline and Inosine are much higher than those of other differential metabolites, while the OR of Arabinosylhypoxanthine is much smaller than those of other differential metabolites, which indicates that 5-Oxoproline, Arabinosylhypoxanthine, and Inosine account for a higher proportion in the model, and thus their values in differential diagnosis of lung cancer and benign pulmonary nodules are also higher, and this finding is consistent with the results obtained in the established model for identifying benign pulmonary nodules and malignant tumors through a single differential metabolite.

In some examples, the biomarker used for determining whether a male patient with pulmonary nodules has lung cancer is Linoleyl carnitine. Similarly, when a model for identifying benign pulmonary nodules and malignant tumors in men through a single differential metabolite is established, it is found that the AUC value of Linoleyl carnitine is 0.867, which is much higher than those of other differential metabolites; and when a model for identifying benign pulmonary nodules and lung cancer in men through a combination of multiple differential metabolites is established, it is found that the absolute value of the model coefficient of Linoleyl carnitine is larger, and its OR is much lower than those of other differential metabolites, which indicates that Linoleyl carnitine has a higher diagnostic value.

In some examples, the biomarker used for determining whether a female patient with pulmonary nodules has lung cancer is one or a combination of both of 5-Oxoproline and Phenylalanine. Similarly, when a model for identifying benign pulmonary nodules and malignant tumors in women through a single differential metabolite is established, it is found that the AUC values of 5-Oxoproline and Phenylalanine are 0.823 and 0.702, which are relatively larger; and when a model for identifying benign pulmonary nodules and lung cancer in men through a combination of multiple differential metabolites is established, it is found that the model coefficients and OR values of 5-Oxoproline and Phenylalanine are much higher than those of other differential metabolites, which indicates that 5-Oxoproline and Phenylalanine have higher diagnostic values.

The invention provides a detection method for diagnosing lung cancer, which includes detecting a biomarker in a blood or serum sample, wherein the biomarker is selected from one or more of: 2-trans,4-cis-Decadienoylcarnitine, Octanoylcarnitine, Decanoylcarnitine, 2-Octenoylcarnitine, Hexanoylcarnitine, 3-hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine, N6,N6,N6-Trimethylysine, Kynurenine, cis-5-Tetradecenoylcarnitine, Docosahexaenoic acid, Choline Sulfate, Dihydrothymine, 17-Hydroxypregnenolone sulfate, Pregnenolone sulfate, Tiglylcarnitine, Propionylcarnitine, 3-hydroxybutyrylcarnitine, Oxindole, Nicotine, Ergothioneine, Phenylacetylglutamine, Citrulline, Lysine, Aminocaproic acid, Methylimidazoleacetic acid.

In some embodiments, the biomarker is selected from one or more of: Decanoylcarnitine, 17-Hydroxypregnenolone sulfate, Pregnenolone sulfate, Tiglylcarnitine, Oxindole, Phenylacetylglutamine, Aminocaproic acid, Methylimidazoleacetic acid.

In some embodiments, the biomarker is selected from one or more of: 2-trans,4-cis-Decadienoylcarnitine, Octanoylcarnitine, Decanoylcarnitine, 2-Octenoylcarnitine, Hexanoylcarnitine, 3-hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine. These biomarkers have significant differences between the patient with lung cancer and healthy people and between the patient with lung cancer and the patient with benign pulmonary nodules, which indicates that they are closely related to lung cancer and are not affected by the presence of benign pulmonary nodules. Therefore, they can be used for differential diagnosis of or distinguishing between lung cancer and benign pulmonary nodules, and can also be used for differential diagnosis of or distinguishing between individuals with lung cancer and healthy individuals (without nodules).

In some embodiments, when it is diagnosed whether an individual without pulmonary nodules has lung cancer, the biomarker is selected from one or more of: 2-trans,4-cis-Decadienoylcarnitine, Octanoylcarnitine, Decanoylcarnitine, 2-Octenoylcarnitine, Hexanoylcarnitine, 3-hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine, N6,N6,N6-Trimethylysine, Tiglylcarnitine, Propionylcarnitine, 3-hydroxybutyrylcarnitine, Oxindole, Nicotine, Ergothioneine, Phenylacetylglutamine, Citrulline, Lysine, Aminocaproic acid, Methylimidazoleacetic acid, wherein the biomarkers N6,N6,N6-Trimethylysine, Tiglylcarnitine, Propionylcarnitine, 3-hydroxybutyrylcarnitine, Oxindole, Nicotine, Ergothioneine, Phenylacetylglutamine, Citrulline, Lysine, Aminocaproic acid, Methylimidazoleacetic acid have significant differences between the patient with lung cancer and healthy people (without nodules), but have no significant difference between the patient with lung cancer and the patient with benign pulmonary nodules, which indicates that these biomarkers are preferred and specific biomarkers to distinguish the patient with lung cancer from healthy people (without nodules).

In some embodiments, when it is diagnosed whether an individual with pulmonary nodules has lung cancer, the biomarker is selected from one or more of: 2-trans,4-cis-Decadienoylcarnitine, Octanoylcarnitine, Decanoylcarnitine, 2-Octenoylcarnitine, Hexanoylcarnitine, 3-hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine, Kynurenine, cis-5-Tetradecenoylcarnitine, Docosahexaenoic acid, Choline Sulfate, Dihydrothymine, 17-Hydroxypregnenolone sulfate, Pregnenolone sulfate, wherein the biomarkers Kynurenine, cis-5-Tetradecenoylcarnitine, Docosahexaenoic acid, Choline Sulfate, Dihydrothymine, 17-Hydroxypregnenolone sulfate, Pregnenolone sulfate.

These biomarkers have significant differences between lung cancer and benign pulmonary nodules, but have no significant difference between the patient with lung cancer and healthy people (without nodules), which indicates that these biomarkers are preferred and specific biomarkers to distinguish lung cancer from benign pulmonary nodules. These biomarkers have more practical significance. The possibility of further detection of canceration is only possible when nodules are generally found in the process of physical examination or diagnosis. At this time, apart from routine puncture biopsy, an effective way of preliminary screening is to conduct preliminary screening by detecting whether there are changes or abnormalities, such as significant changes, in one or more of the aforementioned markers in a blood sample.

The invention establishes a model for identifying between the patient with lung cancer and healthy (without nodules) people or between the patient with lung cancer and the patient with benign pulmonary nodules by a combination of multiple biomarkers.

In some embodiments, when the model is used for distinguishing or identifying the patient with lung cancer from the patient with benign pulmonary nodules, or for identifying whether a patient with pulmonary nodules has lung cancer, the model includes one or more of the following:

model A: In[P/(1−P)]=2.29×M1+1.02×M2+0.64×M3+0.62×M4+0.47×M5+0.42×M6+0.38×M7+0.26×M8+0.05×M9+0.03×M10−0.05×M11−0.12×M12−0.16×M13−0.17×M14−0.36×M15−0.4×M16−0.45×M17−0.46×M18−0.47×M19−0.53×M20−0.55×M21−0.79×M22−0.95×M23−1.02×M24−1.19×M25−1.52×M26−1.88×M27+4.01, wherein M1-M27 are respectively relative abundances of Hypoxanthine, Alanine, 2-Ketobutyric acid, 2-trans,4-cis-Decadienoylcarnitine, Xanthine, 17-Hydroxypregnenolone sulfate, Dihydrothymine, Octanoylcarnitine, Lactic acid, Pregnenolone sulfate, 3-Chlorotyrosine, Cyclohexaneacetic acid, Choline Sulfate, Trimethylamine N-oxide, 2-Octenoylcarnitine, 1-Methylnicotinamide, Serotonin, Docosahexaenoic acid, Decanoylcarnitine, alpha-Eleostearic acid, Homo-L-arginine, Pyruvic acid, 3-hydroxydecanoylcarnitine, Ecgonine, Kynurenine, Ethyl 3-oxohexanoate, Arabinosylhypoxanthine;

model B: In[P/(1−P)]=1.11×M1+0.25×M2+0.13×M3+0.09×M4+0.05×M5−0.01×M6−0.02×M7−0.02×M8−0.04×M9−0.11×M10−0.12×M11−0.19×M12−0.3×M13−0.34×M14−0.45×M15−0.46×M16−0.64×M17−0.68×M18−0.95×M19+2.17, wherein M1-M19 are respectively relative abundances of Hypoxanthine, Alanine, 2-Ketobutyric acid, 17-Hydroxypregnenolone sulfate, Dihydrothymine, Trimethylamine N-oxide, Serotonin, 3-Chlorotyrosine, Hippuric acid, Docosahexaenoic acid, 2-Octenoylcarnitine, 1-Methylnicotinamide, Homo-L-arginine, alpha-Eleostearic acid, Kynurenine, 3-hydroxydecanoyl carnitine, Ecgonine, Ethyl 3-oxohexanoate, Arabinosylhypoxanthine;

model C: In[P/(1−P)]=1.73×M1+0.72×M2+0.31×M3+0.29×M4+0.23×M5+0.15×M6−0.05×M7−0.07×M8−0.07×M9−0.09×M10−0.09×M11−0.16×M12−0.26×M13−0.26×M14−0.27×M15−0.29×M16−0.43×M17−0.45×M18−0.56×M19−0.75×M20−0.87×M21−1.15×M22−1.41×M23+3.07, wherein M1-M23 are respectively relative abundances of Hypoxanthine, Alanine, 2-Ketobutyric acid, 17-Hydroxypregnenolone sulfate, Dihydrothymine, Xanthine, 3-Chlorotyrosine, Cyclohexaneacetic acid, Choline Sulfate, Decanoylcarnitine, Trimethylamine N-oxide, 2-Octenoylcarnitine, PyruMic acid, Docosahexaenoic acid, 1-Methylnicotinamide, Serotonin, Homo-L-arginine, alpha-Eleostearic acid, 3-hydroxydecanoyl carnitine, Ecgonine, Kynurenine, Ethyl 3-oxohexanoate, Arabinosylhypoxanthine;

model D: In[P/(1−P)]=2.35×M1+1.03×M2+0.71×M3+0.68×M4+0.48×M5+0.45×M6+0.42×M7+0.39×M8+0.16×M9+0.03×M10−0.05×M11−0.12×M12−0.17×M13−0.17×M14−0.22×M15−0.39×M16−0.43×M17−0.46×M18−0.49×M19−0.54×M20−0.54×M21−0.57×M22−0.89×M23−0.97×M24−1.07×M25−1.23×M26−1.56×M27−1.93×M28+4.16, wherein M1-M28 are respectively relative abundances of Hypoxanthine, Alanine, 2-Ketobutyric acid, 2-trans,4-cis-Decadienoylcarnitine, Xanthine, Octanoylcarnitine, 17-Hydroxypregnenolone sulfate, Dihydrothymine, Lactic acid, Pregnenolone sulfate, 3-Chlorotyrosine, Cyclohexaneacetic acid, Choline Sulfate, Trimethylamine N-oxide, Hexanoylcarnitine, 2-Octenoylcarnitine, 1-Methylnicotinamide, Serotonin, Docosahexaenoic acid, Decanoylcarnitine, alpha-Eleostearic acid, Homo-L-arginine, Pyruvic acid, 3-hydroxydecanoyl carnitine, Ecgonine, Kynurenine, Ethyl 3-oxohexanoate, Arabinosylhypoxanthine;

wherein, the cut-off values of models A to D are 0.455, 0.511, 0.452 and 0.458, respectively. The measured relative abundance of each biomarker in the blood sample is substituted into the model equation to calculate a P value. When the P value is greater than the cut-off value, it means that the possibility of the blood sample coming from a patient with lung cancer is high; and when the P value is less than the cut-off value, it means that the possibility of the blood sample from an individual with benign pulmonary nodules is high.

In some embodiments, when the model is used for distinguishing or identifying the patient with lung cancer from the healthy (without nodules) individual, the model includes one or more of the following:

model E: In[P/(1−P)]=1.41×V1+0.26×V2+0.04×V3−0.01×V4−0.05×V5−0.09×V6−0.19×V7−0.2×V8−0.32×V9−0.34×V10−0.4×V11−0.48×V12−0.55×V13−0.64×V14−1.07×V15−1.58×V16+3.44, V1-V16 are respectively relative abundances of Hypoxanthine, Alanine, 2-Ketobutyric acid, 3-hydroxybutyrylcarnitine, Nicotine, Hippuric acid, Citrulline, Trimethylamine N-oxide, alpha-Eleostearic acid, 1-Methylnicotinamide, 3-hydroxydecanoylcarnitine, Ecgonine, Ethyl 3-oxohexanoate, 2-trans,4-cis-Decadienoylcarnitine, Arabinosylhypoxanthine, Lysine;

model F: In[P/(1−P)]=1.51×V1+0.29×V2+0.06×V3−0.03×V4−0.03×V5−0.03×V6−0.07×V7−0.1×V8−0.21×V9−0.22×V10−0.33×V11−0.35×V12−0.39×V13−0.51×V14−0.59×V15−0.63×V16−1.12×V17−1.69×V18+3.65, wherein V1-V18 are respectively relative abundances of

Hypoxanthine, Alanine, 2-Ketobutyric acid, 3-hydroxybutyryl carnitine, Decanoylcarnitine, Ergothioneine, Nicotine, Hippuric acid, Trimethylamine N-oxide, Citrulline, alpha-Eleostearic acid, 1-Methylnicotinamide, 3-hydroxydecanoyl carnitine, Ecgonine, Ethyl 3-oxohexanoate, 2-trans,4-cis-Decadienoylcarnitine, Arabinosylhypoxanthine, Lysine;

model G: In[P/(1−P)]=1.67×V1+0.34×V2+0.1×V3+0.01×V4−0.08×V5−0.08×V6−0.09×V7−0.1×V8−0.12×V9−0.23×V10−0.27×V11−0.36×V12−0.36×V13−0.38×V14−0.56×V15−0.61×V16−0.66×V17−1.2×V18−1.89×V19+4, wherein V1-V19 are respectively relative abundances of

Hypoxanthine, Alanine, 2-Ketobutyric acid, Aminocaproic acid, 3-hydroxybutyryl carnitine, Ergothioneine, Decanoylcarnitine, Nicotine, Hippuric acid, Trimethylamine N-oxide, Citrulline, 3-hydroxydecanoyl carnitine, alpha-Eleostearic acid, 1-Methylnicotinamide, Ecgonine, 2-trans,4-cis-Decadienoylcarnitine, Ethyl 3-oxohexanoate, Arabinosylhypoxanthine, Lysine;

model H: In[P/(1−P)]=2.03×V1+0.47×V2+0.15×V3+0.09×V4+0.04×V5+0.03×V6+0.01×V7−0.12×V8−0.12×V9−0.13×V10−0.14×V11−0.14×V12−0.27×V13−0.36×V14−0.37×V15−0.37×V16−0.4×V17−0.43×V18−0.59×V19−0.63×V20−0.75×V21−1.37×V22−2.18×V23+4.56, wherein V1-V22 are respectively relative abundances of Hypoxanthine, Alanine, 2-Ketobutyric acid, Tiglylcarnitine, N6,N6,N6-Trimethylysine, Aminocaproic acid, Oxindole, Decanoylcarnitine, Nicotine, Ergothioneine, 3-hydroxybutyryl carnitine, Hippuric acid, Trimethylamine N-oxide, Lactic acid, Citrulline, alpha-Eleostearic acid, 3-hydroxydecanoyl carnitine, 1-Methylnicotinamide, 2-trans,4-cis-Decadienoylcarnitine, Ecgonine, Ethyl 3-oxohexanoate, Arabinosylhypoxanthine, Lysine; or

model I: In[P/(1−P)]=3.08×V1+1.26×V2+0.7×V3+0.64×V4+0.41×V5+0.4×V6+0.38×V7+0.31×V8+0.31×V9+0.1×V10+0.09×V11+0.09×V12+0.04×V13−0.04×V14−0.04×V15−0.07×V16−0.12×V17−0.17×V18−0.24×V19−0.24×V20−0.26×V21−0.31×V22−0.32×V23−0.44×V24−0.44×V25−0.49×V26−0.53×V27−0.63×V28−0.73×V29−0.79×V30−0.81×V31−0.81×V32−0.85×V33−1.2×V34−1.62×V35−1.72×V36−3.68×V37+7.11, wherein V1-V37 are respectively relative abundances of Hypoxanthine, Octanoylcarnitine, Alanine, 3-hydroxydodecanoyl carnitine, Xanthine, 2-Ketobutyric acid, Oxindole, Tiglylcarnitine, N6,N6,N6-Trimethylysine, Cyclohexaneacetic acid, Aminocaproic acid, Methylimidazoleacetic acid, Homo-L-arginine, Pyruvic acid, 2-Octenoylcarnitine, Propionylcarnitine, Nicotine, Serotonin, Phenylacetylglutamine, Hippuric acid, Ergothioneine, 3-hydroxybutyryl carnitine, alpha-Eleostearic acid, Inosine, Citrulline, Trimethylamine N-oxide, 3-hydroxyoctanoyl carnitine, 1-Methylnicotinamide, 3-hydroxydecanoyl carnitine, Hexanoylcarnitine, Decanoylcarnitine, Ecgonine, 2-trans,4-cis-Decadienoylcarnitine, Ethyl 3-oxohexanoate, Lactic acid, Arabinosylhypoxanthine, Lysine.

The cut-off values of models E to I are 0.520, 0.527, 0.450, 0.466 and 0.456, respectively. The measured relative abundance of each biomarker in the blood sample is substituted into the model equation to calculate a P value. When the P value is greater than the cut-off value, it means that the possibility of the blood sample coming from a patient with lung cancer is high; and when the P value is less than the cut-off value, it means that the possibility of the blood sample from a healthy individual without nodules is high.

Preferably, the relative abundance of the biomarker in the aforementioned model is the measured relative abundance of the biomarker in serum, such as the relative abundance values of these biomarkers in serum as detected by precision analytical instruments including LC-UV, LC-MS, GC-MS, etc; and the relative abundance of these biomarkers in serum as detected by an immune kit, etc.

In some embodiments, these models can be input into the computer system in advance, and when a detection value of a biomarker is obtained, a diagnosis result is obtained through automatic calculation by the computer system. Therefore, the invention can provide a system for distinguishing the patient with lung cancer from the patient with benign pulmonary nodules or healthy people without nodules, which includes an operation module, wherein the operation or calculation module includes a model or model equation established by one or a combination of several ones of the biomarkers.

In some embodiments, the system further includes an output module for outputting the calculation result.

In some embodiments, the system further includes an input module for inputting one or more detection results of the aforementioned biomarkers, and such detection results can be quantitative detection results or qualitative results. The established model here is not a finite model recited in the invention, as long as the biomarkers within the scope of the invention are applied to establish a model for lung cancer diagnosis, the model is within the scope of the invention. In some embodiments, the system further includes a negative control or reference data module.

In some embodiments, an ROC curve is established for each biomarker, and those biomarkers with large areas under the curve (AUC values) can be found, so that one or more biomarkers are chosen to establish a diagnostic model, so as to obtain more reliable diagnostic results. It is generally understood that the reliability of the established model may be higher when more biomarkers are selected. For example, the sensitivity is higher when the accuracy is higher and the specificity is stronger. However, a single biomarker or several important biomarkers can also be selected for diagnosis, or for preliminary screening and detection, which can also provide a reference for diagnosis to a certain extent. In particular, the diagnostic value of a single biomarker may be low, but the diagnostic value of multiple biomarkers newly discovered in the present application or the model established by a combination of the novel biomarkers and known biomarkers may be extremely high. At the same time, it is not that when the diagnostic value of a single biomarker is lower, if it is used for establishing a model of a combination of multiple biomarkers, its weight in the model is lower. For example, in identifying of the patient with lung cancer and healthy people, the AUC value of a single alanine is 0.591 and that of a single 2-trans,4-cis-Decadienoylcarnitine is 0.746, indicating that the diagnostic value of the single 2-trans,4-cis-Decadienoylcarnitine is higher than that of the single alanine. However, in the model A established from multiple biomarkers, the weight coefficients of alanine and 2-trans,4-cis-Decadienoylcarnitine are 1.02 (OR of 2.77) and 0.62 (OR of 1.86), respectively, which means that the value of alanine in the model A is greater than that of 2-trans, 4-cis-Decadienoylcarnitine. That is, when the patient with lung cancer and healthy people are distinguished by the model A, the effect of alanine on the results is greater than that of 2-trans, 4-cis-Decadienoylcarnitine. Similarly, the weight coefficients (or OR) of single biomarkers with small AUC values such as serotonin and 2-butanoic acid in the model established from multiple biomarkers may be higher than those of single biomarkers with large AUC values, which will not be listed one by one here. This further shows that: the diagnostic value of even a biomarker with a very small AUC value cannot be denied, especially that it can be used in combination with other biomarkers and may play a major role in the combined model.

A fourth aspect of the invention provides a kit for screening lung cancer. The kit contains a reagent capable of detecting one or more of the aforementioned biomarkers, which can be a blood treatment reagent, such as a reagent for filtering and extracting the aforementioned biomarkers, and also includes a reagent directly used for directly detecting the presence or present amount of the biomarkers, such as antibodies, antigens or labeling substances.

The invention is advantageous in that:

The method for screening a biomarker for lung cancer is disclosed, a biomarker for distinguishing or identifying the patient with lung cancer from healthy (without nodules) people or the patient with benign pulmonary nodules is provided, and a model for identifying or distinguishing of lung cancer is established. Particularly, the biomarker and model provided by the invention have important clinical diagnostic significance for the differential diagnosis of whether a patient with nodules in the lung has lung cancer.

The invention is advantageous in that: in the invention, small molecular differential metabolites are screened out by utilizing the method of serum metabonomics, and used as biomarkers for the differential diagnosis of lung cancer, which can be used for distinguishing a population with lung cancer from a healthy population and distinguishing a patient with lung cancer from a patient with benign pulmonary nodules, and biomarkers suitable for diagnosis of lung cancer in different genders are further selected and used according to gender. Furthermore, the invention further provides a model for accurate differential diagnosis between lung cancer and benign pulmonary nodules.

Diagnostic Method

A fourth aspect of the invention provides a method for diagnosing lung cancer, which includes detecting the presence or quantity of the aforementioned biomarker in a blood sample, so as to determine the presence of lung cancer or the possibility of suffering from lung cancer.

In some embodiments, the present number is a result obtained by comparing with that in the negative blood sample. In some embodiments, the blood sample is a serum sample.

In some embodiments, the method of diagnosing lung cancer includes a method of screening patients with lung cancer from a healthy population; or alternatively, screening patients with lung cancer from patients with pulmonary nodules; or alternatively screening patients with lung cancer from male healthy people without pulmonary nodules, or alternatively screening patients with lung cancer from male patients with pulmonary nodules; or alternatively screening patients with lung cancer from healthy female people without pulmonary nodules, or alternatively screening patients with lung cancer from female patients with pulmonary nodules. One or more of the biomarkers targeted by these different methods can be selected from the aforementioned markers of the invention.

Such specific diagnosis or detection methods all can adopt existing conventional methods, e.g., a liquid phase detection method, a mass spectrometry method, a gas phase or liquid phase combined with mass spectrometry method, or an immunization method. The immune method includes an enzyme-linked immunosorbent assay, a dry chemical method, a dry test strip method, or an electrochemical method.

Diagnostic Device or Kit

In another aspect of the present invention, provided is a kit for detecting lung cancer, which includes a reagent capable of detecting one or more of the aforementioned biomarkers, which may be a blood treatment reagent, such as a reagent for filtering and extracting the aforementioned biomarkers; and further includes a reagent directly used for detecting the presence or present quantity of biomarkers, e.g., an antibody, antigen or labeling substance.

Metabolic Pathway

The metabolic pathways in which the aforementioned metabolites involve include glycolysis, and cycles of fatty acids, carnitine, amino acids, purine, nicotine, heme, sex hormone, vitamins and tricarboxylic acids.

Therefore, on the other hand, the invention provides use of a biomarker in preparation of a reagent for diagnosing lung cancer, wherein the described biomarkers are derived from one or more of the following metabolic pathways including: glycolysis, and cycles of fatty acids, carnitine, amino acids, purines, nicotine, heme, sex hormones, vitamins and tricarboxylic acid. it is shown through the invention that, the changes of the substances involved in the aforementioned metabolic pathways are closely related to the occurrence of lung cancer, and the degree of this correlation shows a significant difference. The changes of the metabolic pathway substances may be an increase relative to normal, or a decrease relative to normal. Here, the normal refers to a healthy population without nodules or people with benign nodules. Although the changes of some specific compounds found in the invention are presented with relevance to the occurrence of lung cancer, it does not mean that other specific compounds produced by abnormalities in these metabolic pathways are not presented with relevance to the occurrence of lung cancer. In other words, when it is necessary to diagnose or prevent lung cancer, we can first start with a metabolic pathway, and then look for specific compounds or substances involved in the metabolic pathway to find novel compounds, which thus can be used for diagnosing whether lung cancer occurs or for prevention and treatment.

In some embodiments, one of or a combination of several biomarkers mentioned above can be used for diagnosis of lung cancer, and when two or more of the biomarkers are used, the diagnostic effect will be better than that of a single serum marker.

### BRIEF DESCRIPTION OF DRAWINGS

FIG. 1 is an analysis flow chart;

FIG. 2A is a total ion current diagram and FIG. 2B is a mass spectrum diagram;

FIG. 3 is a diagram showing PLS-DA statistical results of three groups, a group of a population with lung cancer, a group of a population with benign pulmonary nodules and a group of healthy population (−ESI: negative spectrum; and +ESI: normal spectrum);

FIG. 4 is a diagram showing PLS-DA statistical results of the group of a population with lung cancer and the group of healthy population (-ESI: negative spectrum; and +ESI: normal spectrum);

FIG. 5 is a diagram showing PLS-DA statistical results of the group of a population with lung cancer and the a group of the population with benign pulmonary nodules (−ESI: negative spectrum; and +ESI: normal spectrum);

FIG. 6 shows a differential metabolite that is common in the patient with lung cancer and the patient with benign pulmonary nodules in men and women, and differential metabolites specific to the patient with lung cancer and the patient with benign pulmonary nodules in men or women.

FIG. 7 shows an ROC curve of model A-1;

FIG. 8 shows an ROC curve of model B-2;

FIG. 9 shows an ROC curve of model C-3;

FIG. 10 shows an ROC curve of model D-4;

FIG. 11 shows the prediction results for lung cancer and benign pulmonary nodules in model A-1;

FIG. 12 is a diagram showing PLS-DA statistical results of normal spectrum (+ESI) and negative spectrum (−ESI) of three groups, i.e., the group of a population with lung cancer, the group of the population with benign pulmonary nodules and the group of healthy people (−ESL negative spectrum; and +ESI: normal spectrum);

FIG. 13 shows an ROC curve of model A;

FIG. 14 shows an ROC curve of model B;

FIG. 15 shows an ROC curve of model C;

FIG. 16 shows an ROC curve of model D;

FIG. 17 shows an ROC curve of model E;

FIG. 18 shows an ROC curve of model F;

FIG. 19 shows an ROC curve of model G;

FIG. 20 shows an ROC curve of model H; and

FIG. 21 shows an ROC curve of model I;

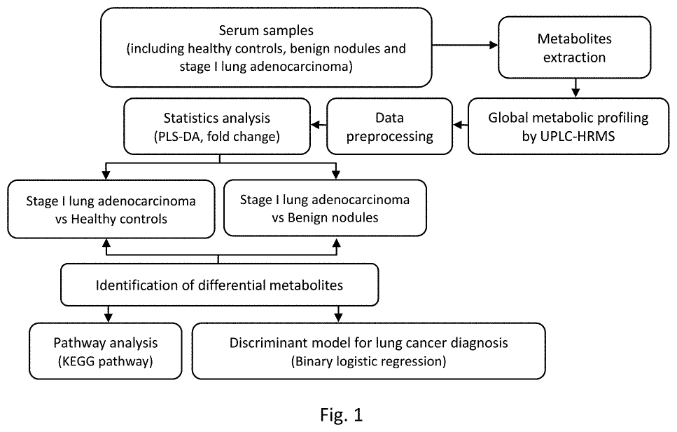

### DETAILED DESCRIPTION OF THE INVENTION

(1) Diagnosis or Detection

Here diagnosis or detection refers to detecting or assaying a biomarker in a sample, or detecting the content of a target biomarker, e.g., absolute content or relative content, and then explaining whether an individual from which the sample is provided has a certain disease or the possibility of suffering from a certain disease through the presence or quantity of the target biomarker.

Here the meanings of diagnosis and detection can be used interchangeably. The result of this detection or diagnosis cannot be directly used as the direct result of suffering from the disease, but an intermediate result. If it is wanted to obtain a direct result, a certain disease can only be confirmed further by means of other auxiliary means such as pathology or anatomy. For example, the invention provides a variety of novel biomarkers with relevance to lung cancer, and changes in the contents of these biomarkers are directly related to whether suffering from lung cancer.

(2) Relationship Between Marker and Lung Cancer

Here the relationship means that the appearance of a certain biomarker in a sample or the change of its content is directly related to a specific disease. For example the relative increase or decrease of its content indicates that the possibility of suffering from this disease is higher than that of healthy people.

If multiple different markers are found in a sample at the same time or their contents changes relatively, it indicates that the possibility of suffering from this disease is higher than that of healthy people. That is, among the species of markers, some markers have a stronger correlation with suffering from the disease, while some markers have a weaker correlation with suffering from the disease, or even some markers have no correlation with a specific disease. One or more of those markers with strong correlation can be used as biomarkers for diagnosing the disease, and those with weak correlation can be combined with strong markers to diagnose certain diseases, thereby increasing the accuracy of detection results.

For the plurality of biomarkers in the serum found by the invention, these biomarkers all can be used for distinguishing the population with lung cancer from healthy people or population with pulmonary nodules. Here, the marker can be used as a single marker for direct detection or diagnosis. Selection of such a marker indicates that the relative change in the content of the marker has a strong correlation with lung cancer. Of course, it can be understood that one or more markers with strong correlation with lung cancer can be selected for simultaneous detection. The normal understanding is that in some embodiments, the selection of highly correlated biomarkers for detection or diagnosis can achieve certain standard accuracy, such as the accuracy of 60%, 65%, 70%, 80%, 85%, 90% or 95%, then it can show that these biomarkers can obtain an intermediate value for diagnosis of a certain disease, but it does not mean that it can be directly confirmed to have the disease. For example, in the invention, among the biomarkers in Tables 2-9, the biomarker with higher VIP value can be selected as a marker for diagnosing whether suffering from lung cancer, or as a marker for screening out the population with lung cancer from healthy population or the population with pulmonary nodules, and here the population includes both people without gender differences and people with gender differences.

Of course, the one with the higher ROC value can also be selected as a diagnostic marker. The so-called strong and weak are generally confirmed by some algorithms, such as the contribution rate of the marker to lung cancer or weight analysis thereof. Such calculation methods can be significance analysis (p value or FDR value) and Fold change. Multivariate statistical analysis mainly includes principal component analysis (PCA), partial least squares discriminant analysis (PLS-DA) and orthogonal partial least squares discriminant analysis (OPLS-DA). Of course, it also includes other methods, e.g., ROC analysis and so on. Of course, other model prediction methods are also possible. During specific selection of a biomarker, the marker disclosed by the invention can be selected, or other well-known markers can be selected or used in combination.

In order to describe the invention more specifically, the technical solution of the invention will be described in detail below with reference to the accompanying drawings and specific examples. These explanations only show how the invention is implemented, but do not define the specific scope of the invention. The scope of the invention is defined in the claims.

### EXAMPLE 1

### Collection of Serum Samples

Serum samples were collected from patients of different genders and ages and healthy people. In this study, samples of men and women aged between 38-78 were collected, including three groups of serum samples from patients with lung cancer (138 cases), patients with benign pulmonary nodules (170 cases) and healthy people (174 cases), which were matched according to gender and age.

### EXAMPLE 2

### Extraction of Serum Metabolites

Serum metabolites were extracted by a three-phase extraction method of methyl tert-butyl ether:methanol:water (10:3:2.5, v/v/v). The specific operation was as follows: (1) the serum sample was placed on ice and completely thawed, 50 uL of the sample was taken into a 1.5 mL EP tube, added with 225 μL of frozen methanol, and subjected to vortex for 30 seconds; (2) it was added with 750 μL of frozen MTBE, subjected to vortex for 30 seconds, and shaken on ice at 400 rpm for 1 hour; (3) it was then added with 188 μL of pure water and subjected to vortex for 1 minute; (4) it was centrifuged at 15,000 rcf for 10 minutes at 4° C.; and (5) upon centrifugation, 125 μL of the subnatant was taken into an EP tube and spin-dried with a vacuum freeze dryer, and all the dry samples of serum metabolites were stored in a refrigerator at −80° C. until testing.

Considering that there might be batch errors in sample pretreatment, in this study, processing of each batch of experimental samples was conducted simultaneously with the processing of one Reference serum for subsequent data correction. The Reference serum sample was prepared by mixing sera from 100 healthy people (healthy people referred to the people whose blood pressure, blood sugar and blood routine were all normal and that had no hepatitis B virus, and of which the physical examination results showed no obvious diseases, so that they did not need to see a doctor for treatment currently). The men and women from which the sera of 100 healthy people were derived were of the equal number, and were aged between 40-55. The subjects needed to fast overnight and forbid taking drugs 72 hours before blood collection, and individuals with past disease history and body mass indexes (BM's) outside the 95th percentile were excluded. The mixed serum was sub-packaged in 50 μL per portion and stored in a refrigerator at −80° C.

### EXAMPLE 3

### Detection of Extracted Serum Metabolites and Data Preprocessing

(1) Reconstitution of serum metabolites: the dry extract of serum metabolites was added with 120 μL of a reconstitution solvent (acetonitrile:water=4:1), subjected to vortex for 5 minutes, and then centrifuges at 4° C. for 15,000×g for 10 minutes, and 100 μL of the supernatant was taken into a liner tube to prepare a sample to be tested.

(2) QC sample: each 10 μL of the serum samples to be tested from patients with lung cancer, patients with benign pulmonary nodules and healthy people was taken, subjected to vortex, and mixed evenly with shaking to prepare a QC sample.

(3) Sample detection method: detection was conducted with liquid chromatography-high resolution mass spectrometry (LC-HRMS).

I. Liquid Chromatography Conditions

Chromatographic Column: BEH Amide (100×2.1 mm, 1.7 μm).

Mobile phase: in positive mode, phase A was acetonitrile; water=95:5 (10 mM ammonium acetate, 0.1% formic acid), and phase B was acetonitrile:water=50:50 (10 mM ammonium acetate, 0.1% formic acid); and in negative mode, phase A was acetonitrile:water=95:5 (10 mM ammonium acetate, pH=9.0, adjusted by aqueous ammonia), and phase B was acetonitrile: water=50:50 (10 mM ammonium acetate, pH=9.0, adjusted by aqueous ammonia).

The elution gradient was shown in Table 1 below:

TABLE 1
Elution gradient of LC-HRMS mobile phase

| Time (min) | Flow rate (mL/min) | Phase A | Phase B |
|---|---|---|---|
| 0.0 | 0.30 | 98 | 2 |
| 0.50 | 0.30 | 98 | 2 |
| 12.0 | 0.30 | 50 | 50 |
| 14.0 | 0.30 | 2 | 98 |
| 16.0 | 0.30 | 2 | 98 |
| 16.1 | 0.30 | 98 | 2 |
| 20.0 | 0.30 | 98 | 2 |

II. Mass Spectrometry Conditions

The model of a mass spectrometer was Q Exactive (Thermo Fisher Scientific Company, USA), and qualitative analysis was carried out by employing an electrospray ion source (ESI), a positive and negative Fullscan mode (Full Scan) and a data dependent scan mode (ddMS2). The spray voltage was+3,800/-3,200 V; the atomization temperature was 350° C.; high-purity nitrogen was used as sheath gas and auxiliary gas, and the parameters were set to 40 arb and 10 arb; respectively; the temperature of ion transfer tube was 320° C.; the mass scanning range was 70-1,050 m/z; the primary scan resolution was 70,000 FWHM, and the secondary scan resolution was 35,000 FWHM.

III. Injecting Method

Before each detection, six syringe volumes of the QC sample were injected to stabilize the detection system. The serum sample was injected in a random manner, in which testing of one syringe volume of the QC sample was inserted every injection of 10 syringe volumes of the serum samples. The first syringe and last syringe in the detection sequence were both the QC sample. Finally, the QC sample was subjected to full scanning and segmented scanning by ddMS2 for compound identification.

(4) Data Preprocessing

I. Raw Data Matrix

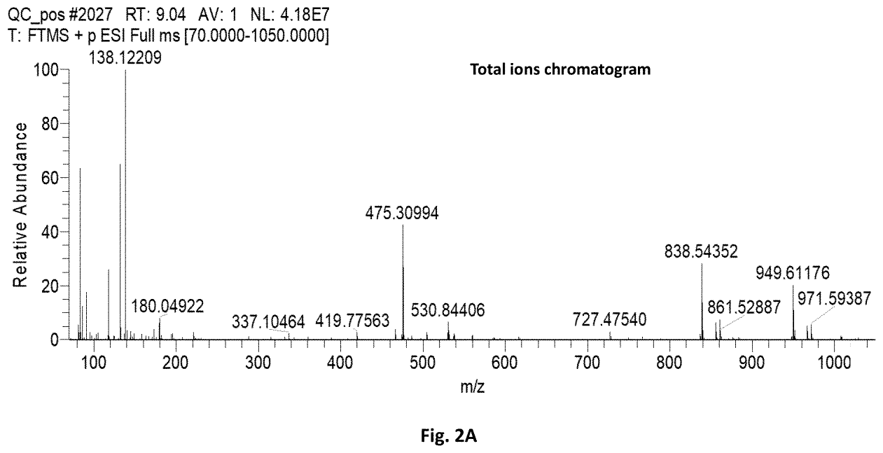

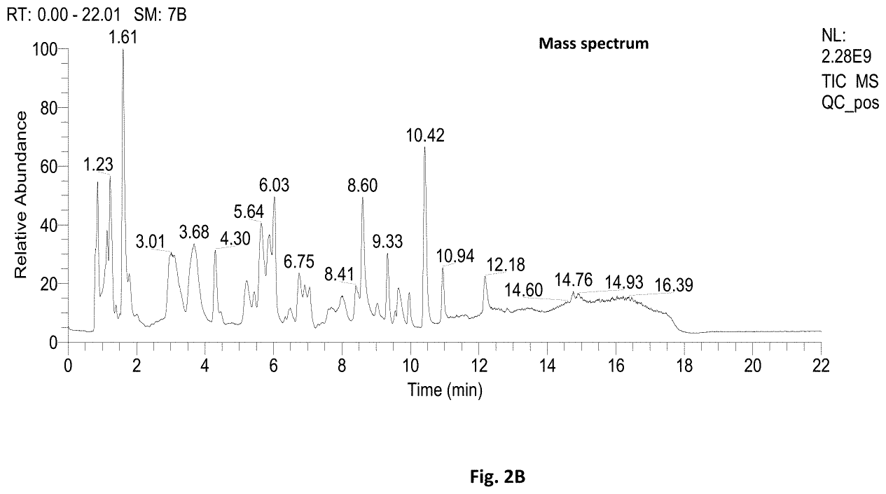

The raw data of each sample included total ion current data and mass spectrum data (as shown in FIG. 2). All the raw data of the sample was imported into Compound Discovery software to get the information of m/z ions and retention time, and the database (mzCloud and Chemspider) was searched to get the identification results of the compound. Further, according to the information of m/z ions and retention time, the chromatographic integration of each sample was carried out by using Tracefinder software, so as to obtain more accurate peak area information. Finally, a two-dimensional data matrix containing characteristic ions (a combination of m/z ions and retention time) and contents thereof (peak area) was obtained for each sample.

II. Excision and Interpolation of Data Missing Values

There were often data missing values in the original data matrix of metabonomics, which were mainly related to the detection of background noise, peak extraction and peak alignment methods of mass spectrometry, etc. Too many zero or missing values would bring difficulties to downstream analysis. Therefore, characteristic ions with missing values greater than 50% in all samples were generally excised, and the missing values of other compounds were interpolated. In this study, MetaboAnalyst 5.0 analysis software was used for processing the missing values, and a K-Nearest Neighbours (KNN) manner was selected for interpolation.

III. Data Correction and Filtering

A large amount of sample pretreatment was inevitably limited by the throughput of experimental treatment, so it was necessary to carry out sample pretreatment in batches. However, due to the multifarious types of metabolites, large differences in physical and chemical properties, and expensive isotope internal standards, it was difficult to choose an appropriate isotope internal standard that could meet the full coverage. Aiming at this problem, this study selected a reference serum that is processed simultaneously with the batch processing as a natural “like internal standard” to correct batch errors caused by pretreatment. That was, the original data of the experimental samples of each pretreatment batch was normalized based on the data of the Reference serum of the corresponding batch to obtain the relative abundance of each characteristic ion, and the characteristic ion with RSD>30% in the QC sample was deleted to obtain the final analysis data matrix.

### EXAMPLE 4

### Samples were Grouped by Partial Least Squares Discriminant Analysis, So as to Screen Out Differential Metabolites in Different Groups in Connection with Significance Analysis

Metabonomics generally adopted the combination of univariate analysis and multivariate statistical analysis to screen differential metabolites, in which the univariate analysis mainly included significance analysis (p value or FDR value) and Fold change of characteristic ions in different groups, while the multivariate statistical analysis mainly included principal component analysis (PCA), partial least squares discriminant analysis (PLS-DA) and orthogonal partial least squares discriminant analysis (OPLS-DA).

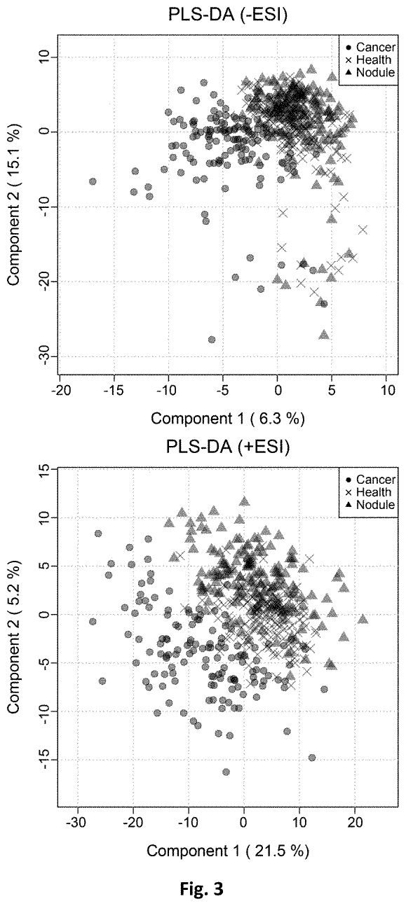

Before statistical analysis, the data should be properly normalized, transformed and scaled. In this study, MetaboAnalyst 5.0 analysis software was used for statistical analysis, and data normalization by the sum, Log transformation and Auto scaling were carried out. Partial least squares discriminant analysis (PLS-DA) was performed on the three groups of lung cancer, benign pulmonary nodules and healthy people (as shown in FIG. 3), and obvious grouping results were obtained.

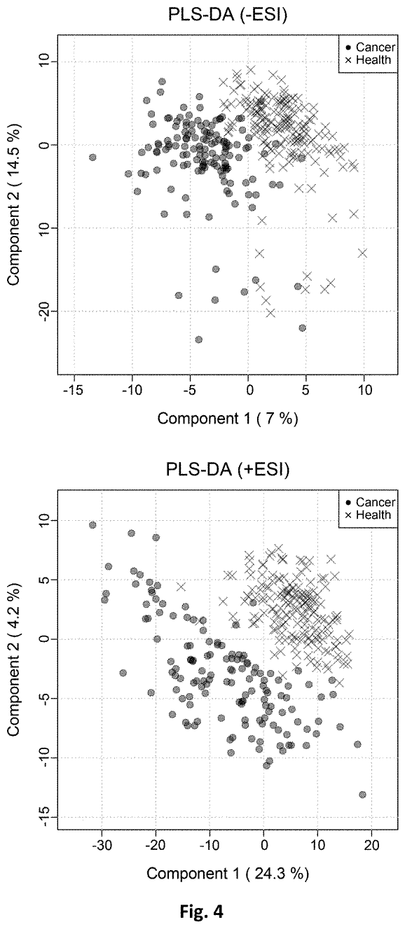

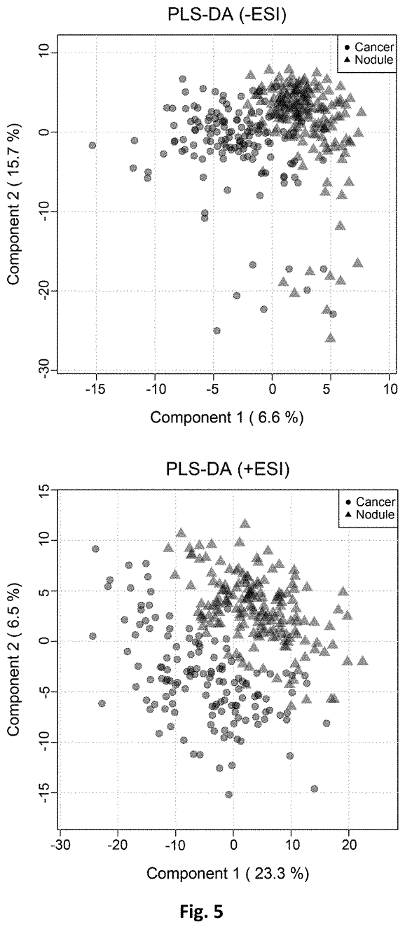

Further, PLS-DA analysis between two groups of patients with lung cancer and healthy people, patients with lung cancer and patients with benign pulmonary nodules was carried out (as shown in FIGS. 4 and 5), variable importance for the projection (VIP) was calculated to measure the influence strength and explanatory power of the expression pattern of each metabolite on the discrimination of classification of each group of samples, a Wilcoxon rank sum test was further carried out to obtain the corrected p value (FDR), and the fold change (FC) between two groups was calculated according to the intra-group average.

According to the screening criteria of the differential metabolite: (1) VIP>1; (2) when FDR<0.05, i.e. VIP>1 and FDR<0.05, it was judged that there was a significant difference in the metabolite between the two groups, and the metabolite was the differential metabolite between the two groups. Moreover, in the process of screening the differential metabolites, it was found that the differential metabolites for different genders were different, so the differential metabolites were further differentiated according to gender.

The main significant differential metabolites found by the invention were:

1. differential metabolites between the group of patients with lung cancer and the group of healthy people were shown in Table 2 below.

TABLE 2
Differential metabolites between lung cancer samples and healthy
samples (without nodules)

| | | | | | Related metabolic |
|---|---|---|---|---|---|
| No. | Name | FDR | VIP | FC | pathway |
| 1 | Hippuric acid | 4.02E−07 | 1.78 | 0.49 | Phenylalanine metabolism |
| 2 | Hypoxanthine | 1.16E−05 | 1.43 | 1.17 | Purine Metabolism |
| 3 | Serotonin | 1.47E−06 | 1.01 | 0.80 | Tryptophan Metabolism |
| 4 | Lactic acid | 2.77E−03 | 1.15 | 1.11 | Gluconeogenesis, Pyruvate |
| | | | | | Metabolism |
| 5 | 2-Octenoyl- | 1.24E−05 | 1.47 | 0.72 | Lipid metabolism pathway |
| | carnitine | | | | |
| 6 | 2-trans,4-cis- | 2.74E−13 | 1.93 | 0.64 | Lipid metabolism pathway |
| | Decadienoyl- | | | | |
| | carnitine | | | | |
| 7 | 3-hydroxy- | 2.58E−11 | 1.49 | 0.74 | Lipid metabolism pathway |
| | decanoyl | | | | |
| | carnitine | | | | |
| 8 | 3-hydroxy- | 1.73E−09 | 1.96 | 0.70 | Lipid metabolism pathway |
| | dodecanoyl- | | | | |
| | carnitine | | | | |
| 9 | 3-hydroxy- | 1.86E−09 | 1.26 | 0.78 | Lipid metabolism pathway |
| | octanoyl | | | | |
| | carnitine | | | | |
| 10 | Hexanoyl- | 1.14E−06 | 1.16 | 0.75 | Lipid metabolism pathway |
| | carnitine | | | | |
| 11 | Octanoyl- | 1.09E−09 | 1.60 | 0.67 | Lipid metabolism pathway |
| | carnitine | | | | |
| 12 | Xanthine | 3.86E−04 | 1.23 | 1.18 | Purine Metabolism |
| 13 | Arabinosyl- | 2.89E−12 | 2.28 | 0.40 | N/A |
| | hypoxanthine | | | | |
| 14 | Uridine | 5.78E−03 | 1.06 | 0.87 | Pyrimidine Metabolism |
| 15 | Ecgonine | 1.28E−10 | 1.53 | 0.69 | N/A |
| 16 | N-Acetyl- | 1.43E−04 | 1.21 | 1.09 | N/A |
| | L-alanine | | | | |
| 17 | Acetophenone | 5.65E−04 | 1.38 | 0.67 | N/A |
| 18 | Succinic acid | 6.80E−03 | 1.07 | 1.24 | Alanine, aspartate and |
| | semialdehyde | | | | glutamate metabolism, |
| | | | | | Butanoate metabolism |
| 19 | Cyclohexane- | 2.56E−03 | 1.15 | 0.71 | N/A |
| | acetic acid | | | | |
| 20 | Dihydroxy- | 1.42E−03 | 1.26 | 0.74 | N/A |
| | benzoic acid | | | | |
| 21 | Pyruvic acid | 1.15E−04 | 1.37 | 1.26 | Citrate cycle (TCA cycle), |
| | | | | | Pyruvate metabolism |
| 22 | Ethyl 3- | 7.74E−05 | 1.43 | 0.76 | N/A |
| | oxohexanoate | | | | |
| 23 | 2-Ketobutyric | 7.53E−03 | 1.01 | 1.24 | Methionine Metabolism, |
| | acid | | | | Glycine and Serine |
| | | | | | Metabolism, |
| | | | | | Selenoamino Acid |
| | | | | | Metabolism |
| 24 | Methylaceto- | 3.48E−03 | 1.02 | 1.07 | N/A |
| | acetic acid | | | | |
| 25 | Homo-L- | 8.31E−09 | 1.17 | 0.78 | N/A |
| | arginine | | | | |
| 26 | 5-Oxoproline | 5.19E−11 | 1.92 | 1.17 | Glutathione metabolism |
| 27 | 3- | 5.67E−05 | 1.49 | 0.48 | N/A |
| | Chlorotyrosine | | | | |
| 28 | Docosa- | 9.94E−04 | 1.03 | 0.84 | Biosynthesis |
| | hexaenoic | | | | of unsaturated |
| | acid | | | | fatty acids |
| 29 | alpha- | 2.31E−09 | 1.47 | 0.75 | N/A |
| | Eleostearic acid | | | | |

Note:
FC in the table was the multiple ratio of the lung cancer samples to the healthy samples;
and N/A indicated that no relevant metabolic pathway had been found.

2. Different metabolites between group of patients with lung cancer and the group of patients with benign pulmonary nodules were shown in Table 3 below.

TABLE 3
Differential metabolites between the lung cancer (lung malignant tumor)
samples and the samples with benign lung nodules

| | | | | | Related metabolic |
|---|---|---|---|---|---|
| No. | Name | FDR | VIP | FC | pathway |
| 1 | Hippuric acid | 3.55E−05 | 1.64 | 0.59 | Phenylalanine |
| | | | | | metabolism |
| 2 | Hypoxanthine | 7.34E−06 | 1.67 | 1.15 | Purine Metabolism |
| 3 | Trimethylamine | 7.66E−06 | 1.43 | 0.73 | N/A |
| | N-oxide | | | | |
| 4 | 1-Methyl- | 2.85E−07 | 1.70 | 0.71 | Nicotinate and |
| | nicotinamide | | | | Nicotinamide |
| | | | | | Metabolism |
| 5 | Lactic acid | 1.14E−05 | 1.69 | 1.13 | Gluconeogenesis, |
| | | | | | Pyruvate |
| | | | | | Metabolism |
| 6 | Kynurenine | 7.90E−05 | 1.12 | 0.82 | Tryptophan Metabolism |
| 7 | Serotonin | 4.24E−06 | 1.50 | 0.79 | Tryptophan Metabolism |
| 8 | Linoleyl carnitine | 1.66E−05 | 1.23 | 0.78 | Lipid metabolism |
| | | | | | pathway |
| 9 | Acetylcarnitine | 7.11E−04 | 1.10 | 0.92 | Beta Oxidation of |
| | | | | | Very Long Chain |
| | | | | | Fatty Acids |
| 10 | 2-Octenoyl- | 3.91E−06 | 1.41 | 0.70 | Lipid metabolism |
| | carnitine | | | | pathway |
| 11 | 2-trans,4-cis- | 6.96E−05 | 1.39 | 0.77 | Lipid metabolism |
| | Decadienoyl- | | | | pathway |
| | carnitine | | | | |
| 12 | 3-hydroxy- | 8.74E−09 | 1.85 | 0.69 | Lipid metabolism |
| | decanoyl | | | | pathway |
| | carnitine | | | | |
| 13 | 3-hydroxydo- | 2.46E−06 | 1.36 | 0.65 | Lipid metabolism |
| | decanoyl carnitine | | | | pathway |
| 14 | 3-hydroxy- | 1.30E−08 | 1.78 | 0.72 | Lipid metabolism |
| | octanoyl carnitine | | | | pathway |
| 15 | Hexanoyl- | 1.17E−03 | 1.12 | 0.86 | Lipid metabolism |
| | carnitine | | | | pathway |
| 16 | cis-5-Tetra- | 6.74E−03 | 1.08 | 0.82 | Lipid metabolism |
| | decenoylcarnitine | | | | pathway |
| 17 | Octanoylcarnitine | 7.72E−06 | 1.40 | 0.77 | Lipid metabolism |
| | | | | | pathway |
| 18 | Xanthine | 1.03E−03 | 1.36 | 1.14 | Purine Metabolism |
| 19 | Arabinosyl- | 1.44E−13 | 2.66 | 0.34 | N/A |
| | hypoxanthine | | | | |
| 20 | Inosine | 2.94E−12 | 1.84 | 0.39 | Purine Metabolism |
| 21 | Dihydrothymine | 2.99E−03 | 1.05 | 1.17 | Pyrimidine Metabolism |
| 22 | Creatinine | 4.75E−03 | 1.04 | 0.97 | Arginine and |
| | | | | | proline metabolism |
| 23 | Bilirubin | 1.21E−03 | 1.20 | 0.84 | Porphyrin Metabolism |
| 24 | Ecgonine | 8.06E−10 | 1.90 | 0.66 | N/A |
| 25 | Choline Sulfate | 1.04E−06 | 1.20 | 0.78 | N/A |
| 26 | 4-oxo- | 2.40E−05 | 1.42 | 0.70 | Retinol Metabolism |
| | Retinoic acid | | | | |
| 27 | Acetophenone | 5.21E−03 | 1.14 | 0.70 | N/A |
| 28 | Diethylamine | 5.77E−04 | 1.16 | 0.96 | N/A |
| 29 | 7-Methylguanine | 9.88E−05 | 1.35 | 0.92 | N/A |
| 30 | Homo-L-arginine | 1.98E−07 | 1.49 | 0.78 | N/A |
| 31 | N-Acetyl- | 1.26E−04 | 1.37 | 1.06 | N/A |
| | L-alanine | | | | |
| 32 | 5-Oxoproline | 1.44E−13 | 2.40 | 1.17 | Glutathione metabolism |
| 33 | Citrulline | 2.78E−04 | 1.14 | 0.93 | Arginine and |
| | | | | | Proline Metabolism, |
| | | | | | Aspartate Metabolism, |
| | | | | | Urea Cycle |
| 34 | Glutamine | 2.33E−04 | 1.14 | 0.95 | Pyrimidine Metabolism, |
| | | | | | Glutamate Metabolism |
| 35 | Lysine | 3.04E−04 | 1.04 | 0.95 | Lysine Degradation, |
| | | | | | Biotin Metabolism |
| 36 | 3-Chlorotyrosine | 1.06E−03 | 1.41 | 0.61 | N/A |
| 37 | 2-Pyrrolidone | 2.00E−05 | 1.34 | 1.21 | N/A |
| 38 | Hydroxybutyric | 1.18E−03 | 1.33 | 1.14 | Ketone Body |
| | acid | | | | Metabolism |
| 39 | Succinic acid | 5.96E−03 | 1.15 | 1.21 | Alanine, aspartate |
| | semialdehyde | | | | and glutamate |
| | | | | | metabolism, Butanoate |
| | | | | | metabolism |
| 40 | Cyclohexane- | 2.24E−05 | 1.56 | 0.71 | N/A |
| | acetic acid | | | | |
| 41 | Dihydroxy- | 5.76E−04 | 1.44 | 0.65 | N/A |
| | benzoic acid | | | | |
| 42 | Pyruvic acid | 8.28E−04 | 1.31 | 1.20 | Citrate cycle (TCA |
| | | | | | cycle), Pyruvate |
| | | | | | metabolism |
| 43 | Ethyl | 3.55E−05 | 1.53 | 0.71 | N/A |
| | 3-oxohexanoate | | | | |
| 44 | 2-Ketobutyric | 8.55E−03 | 1.07 | 1.21 | Methionine |
| | acid | | | | Metabolism, Glycine |
| | | | | | and Serine Metabolism, |
| | | | | | Selenoamino Acid |
| | | | | | Metabolism |
| 45 | Docosahexaenoic | 1.14E−05 | 1.52 | 0.74 | Biosynthesis of |
| | acid | | | | unsaturated fatty |
| | | | | | acids |
| 46 | alpha-Eleostearic | 9.95E−07 | 1.45 | 0.74 | N/A |
| | acid | | | | |

Note:
FC in the table was the multiple ratio of the lung cancer samples to the samples with benign pulmonary nodules;
and N/A indicated that no relevant metabolic pathway had been found.

3. differential metabolites between the group of patients with lung cancer and the group of healthy people in men were shown in Table 4 below.

TABLE 4
Differential metabolites between lung cancer samples and healthy
samples (without nodules) in men

| | | | | | Related metabolic |
|---|---|---|---|---|---|
| No. | Name | FDR | VIP | FC | pathway |
| 1 | Carnitine | 6.65E−05 | 1.12 | 0.95 | Beta Oxidation |
| | | | | | of Very Long |
| | | | | | Chain Fatty Acids, |
| | | | | | Carnitine Synthesis |
| 2 | 3b,16a- | 1.35E−02 | 1.13 | 0.75 | Lipid metabolism |
| | Dihydroxy- | | | | pathway |
| | androstenone | | | | |
| | sulfate | | | | |
| 3 | Tyrosine | 1.70E−04 | 1.04 | 0.93 | Tyrosine Metabolism, |
| | | | | | Phenylalanine and |
| | | | | | TyrosineMetabolism, |
| | | | | | Catecholamine |
| | | | | | Biosynthesis |
| 4 | Isoleucine | 2.58E−05 | 1.20 | 0.92 | Valine, leucine |
| | | | | | and isoleucine |
| | | | | | biosynthesis; |
| | | | | | Valine, leucine |
| | | | | | and isoleucine |
| | | | | | degradation |
| 5 | Leucine | 1.80E−05 | 1.11 | 0.95 | Valine, leucine |
| | | | | | and isoleucine |
| | | | | | biosynthesis; |
| | | | | | Valine, leucine |
| | | | | | and isoleucine |
| | | | | | degradation |
| 6 | Lysine | 3.08E−05 | 1.10 | 0.91 | Lysine Degradation, |
| | | | | | Biotin Metabolism |
| 7 | 3-Chloro- | 8.19E−03 | 1.37 | 0.61 | N/A |
| | tyrosine | | | | |
| 8 | Hippuric acid | 1.58E−03 | 1.52 | 0.60 | Phenylalanine |
| | | | | | metabolism |
| 9 | Hypoxanthine | 5.24E−03 | 1.13 | 1.11 | Purine Metabolism |
| 10 | Trimethyl- | 6.11E−06 | 1.21 | 0.62 | N/A |
| | amine N- | | | | |
| | oxide | | | | |
| 11 | 1-Methyl- | 5.22E−04 | 1.02 | 0.80 | Nicotinate and |
| | nicotinamide | | | | Nicotinamide |
| | | | | | Metabolism |
| 12 | Linoleyl | 1.47E−05 | 1.18 | 0.79 | Lipid metabolism |
| | carnitine | | | | pathway |
| 13 | 2-trans,4-cis- | 6.14E−08 | 1.53 | 0.61 | Lipid metabolism |
| | Decadienoyl- | | | | pathway |
| | carnitine | | | | |
| 14 | 3-hydroxy- | 9.50E−06 | 1.29 | 0.74 | Lipid metabolism |
| | decanoyl | | | | pathway |
| | carnitine | | | | |
| 15 | 3-hydroxy- | 4.70E−04 | 1.01 | 0.77 | Lipid metabolism |
| | dodecanoyl | | | | pathway |
| | carnitine | | | | |
| 16 | 3-hydroxy- | 1.72E−05 | 1.23 | 0.77 | Lipid metabolism |
| | octanoyl | | | | pathway |
| | carnitine | | | | |
| 17 | Acetyl- | 9.47E−06 | 1.14 | 0.88 | Beta Oxidation of |
| | carnitine | | | | Very Long Chain |
| | | | | | Fatty Acids |
| 18 | Octanoyl- | 2.70E−05 | 1.13 | 0.71 | Lipid metabolism |
| | carnitine | | | | pathway |
| 19 | Arabinosyl- | 3.64E−06 | 2.11 | 0.38 | N/A |
| | hypoxanthine | | | | |
| 20 | Inosine | 1.81E−05 | 1.14 | 0.47 | Purine Metabolism |
| 21 | Ecgonine | 1.45E−07 | 1.37 | 0.63 | N/A |
| 22 | N-Acetyl- | 8.16E−04 | 1.35 | 1.10 | N/A |
| | L-alanine | | | | |
| 23 | 4-oxo- | 1.66E−06 | 1.26 | 0.52 | Retinol Metabolism |
| | Retinoic acid | | | | |
| 24 | Diethylamine | 8.34E−05 | 1.11 | 0.95 | N/A |
| 25 | 7-Methyl- | 1.81E−05 | 1.23 | 0.89 | N/A |
| | guanine | | | | |
| 26 | Cyclohexane- | 4.77E−02 | 1.07 | 0.78 | N/A |
| | acetic acid | | | | |
| 27 | Pyruvic acid | 4.31E−03 | 1.37 | 1.27 | Citrate cycle |
| | | | | | (TCA cycle), |
| | | | | | Pyruvate metabolism |
| 28 | Ethyl 3- | 1.54E−02 | 1.28 | 0.73 | N/A |
| | oxohexanoate | | | | |
| 29 | Homo-L- | 5.99E−06 | 1.03 | 0.78 | N/A |
| | arginine | | | | |
| 30 | 5-Oxoproline | 5.78E−06 | 1.74 | 1.14 | Glutathione |
| | | | | | metabolism |
| 31 | Glutamine | 1.83E−04 | 1.11 | 0.94 | Pyrimidine |
| | | | | | Metabolism, |
| | | | | | Glutamate Metabolism |
| 32 | Pilocarpine | 3.19E−07 | 1.39 | 0.89 | N/A |
| 33 | Docosa- | 8.12E−04 | 1.43 | 0.67 | Biosynthesis of |
| | hexaenoic | | | | unsaturated |
| | acid | | | | fatty acids |
| 34 | alpha- | 1.27E−08 | 1.47 | 0.66 | N/A |
| | Eleostearic | | | | |
| | acid | | | | |

Note:
FC in the table was the multiple ratio of the lung cancer samples to the healthy samples in men;
and N/A indicated that no relevant metabolic pathway had been found

4. Different metabolites between group of patients with lung cancer and the group of patients with benign pulmonary nodules in men were shown in Table 5 below.

TABLE 5
Differential metabolites between the lung cancer samples and
the samples with benign lung nodules in men

| | | | | | Related metabolic |
|---|---|---|---|---|---|
| No. | Name | FDR | VIP | FC | pathway |
| 1 | Hippuric acid | 2.15E−02 | 1.20 | 0.68 | Phenylalanine metabolism |
| 2 | Hypoxanthine | 2.96E−04 | 1.46 | 1.09 | Purine Metabolism |
| 3 | Trimethyl- | 6.67E−05 | 1.45 | 0.65 | N/A |
| | amine | | | | |
| | N-oxide | | | | |
| 4 | 1-Methyl- | 1.82E−03 | 1.25 | 0.80 | Nicotinate and |
| | nicotinamide | | | | Nicotinamide |
| | | | | | Metabolism |
| 5 | Linoleyl | 2.09E−11 | 2.33 | 0.57 | Lipid metabolism pathway |
| | carnitine | | | | |
| 6 | 2-trans,4-cis- | 2.25E−05 | 1.62 | 0.68 | Lipid metabolism pathway |
| | Decadienoyl- | | | | |
| | carnitine | | | | |
| 7 | 3-hydroxy- | 3.90E−06 | 1.73 | 0.63 | Lipid metabolism pathway |
| | decanoyl | | | | |
| | carnitine | | | | |
| 8 | 3-hydroxy- | 3.80E−05 | 1.51 | 0.60 | Lipid metabolism pathway |
| | dodecanoyl | | | | |
| | carnitine | | | | |
| 9 | 3-hydroxy- | 1.56E−06 | 1.75 | 0.65 | Lipid metabolism pathway |
| | octanoyl | | | | |
| | carnitine | | | | |
| 10 | Acetyl- | 3.66E−04 | 1.23 | 0.89 | Beta Oxidation |
| | carnitine | | | | of Very Long |
| | | | | | Chain Fatty Acids |
| 11 | Octanoyl- | 1.32E−03 | 1.15 | 0.83 | Lipid metabolism pathway |
| | carnitine | | | | |
| 12 | Arabinosyl- | 8.00E−07 | 2.26 | 0.28 | N/A |
| | hypoxanthine | | | | |
| 13 | Inosine | 2.20E−05 | 1.31 | 0.38 | Purine Metabolism |
| 14 | Ecgonine | 3.74E−08 | 1.96 | 0.59 | N/A |
| 15 | N-Acetyl-L- | 8.00E−06 | 1.86 | 1.09 | N/A |
| | alanine | | | | |
| 16 | 4-oxo- | 4.78E−04 | 1.22 | 0.68 | Retinol Metabolism |
| | Retinoic | | | | |
| | acid | | | | |
| 17 | Diethylamine | 1.86E−03 | 1.11 | 0.96 | N/A |
| 18 | 7-Methyl- | 4.37E−03 | 1.20 | 0.92 | N/A |
| | guanine | | | | |
| 19 | Cyclohexane- | 5.25E−04 | 1.52 | 0.65 | N/A |
| | acetic acid | | | | |
| 20 | Pyruvic acid | 5.04E−04 | 1.46 | 1.20 | Citrate cycle (TCA cycle); |
| | | | | | Pyruvate metabolism |
| 21 | Ethyl 3- | 3.57E−04 | 1.64 | 0.64 | N/A |
| | oxohexanoate | | | | |
| 22 | Homo-L- | 6.23E−04 | 1.12 | 0.78 | N/A |
| | arginine | | | | |
| 23 | 5-Oxoproline | 4.52E−07 | 2.04 | 1.11 | Glutathione metabolism |
| 24 | Glutamine | 1.04E−02 | 1.09 | 0.95 | Pyrimidine Metabolism; |
| | | | | | Glutamate Metabolism |
| 25 | Docosa- | 1.88E−05 | 1.77 | 0.57 | Biosynthesis |
| | hexaenoic | | | | of unsaturated |
| | acid | | | | fatty acids |
| 26 | alpha- | 4.29E−07 | 1.69 | 0.63 | N/A |
| | Eleostearic | | | | |
| | acid | | | | |
| 27 | Lactic acid | 9.78E−04 | 1.48 | 1.09 | Gluconeogenesis, Pyruvate |
| | | | | | Metabolism |
| 28 | 2-Octenoyl- | 6.82E−05 | 1.41 | 0.63 | Lipid metabolism pathway |
| | carnitine | | | | |
| 29 | 3-hydroxy- | 3.80E−04 | 1.11 | 0.83 | Lipid metabolism pathway |
| | butyryl | | | | |
| | carnitine | | | | |
| 30 | Dihydro- | 1.64E−03 | 1.16 | 1.18 | Pyrimidine Metabolism |
| | thymine | | | | |
| 31 | Bilirubin | 1.08E−03 | 1.29 | 0.77 | Porphyrin Metabolism |
| 32 | Nicotine | 1.78E−04 | 1.26 | 0.66 | Nicotine Metabolism |
| | | | | | Pathway |
| 33 | Ergothioneine | 1.82E−03 | 1.02 | 0.74 | Histidine metabolism |
| 34 | Aminoadipic | 6.99E−04 | 1.44 | 1.05 | Lysine Degradation |
| | acid | | | | |
| 35 | N6,N6,N6- | 2.02E−03 | 1.08 | 1.39 | Carnitine Synthesis |
| | Tri- | | | | |
| | methylysine | | | | |

Note:
FC in the table was the multiple ratio of the lung cancer samples to the samples with benign pulmonary nodules in men;
and N/A indicated that no relevant metabolic pathway had been found.

5. differential metabolites between patients with lung cancer and healthy people in women were shown in Table 6 below.

TABLE 6
Differential metabolites between lung cancer samples and healthy
samples (without nodules) in women

| | | | | | Related metabolic |
|---|---|---|---|---|---|
| No. | Name | FDR | VIP | FC | pathway |
| 1 | Carnitine | 1.32E−03 | 1.03 | 0.96 | Beta Oxidation |
| | | | | | of Very Long |
| | | | | | Chain Fatty |
| | | | | | Acids, Carnitine |
| | | | | | Synthesis |
| 2 | Alanine | 1.35E−03 | 1.39 | 1.23 | Alanine Metabolism, Urea |
| | | | | | Cycle, Selenoamino Acid |
| | | | | | Metabolism |
| 3 | Linoleyl | 6.64E−04 | 1.18 | 1.58 | Lipid metabolism pathway |
| | carnitine | | | | |
| 4 | Propionyl- | 5.10E−04 | 1.08 | 0.89 | Oxidation of |
| | carnitine | | | | Branched Chain |
| | | | | | Fatty Acids |
| 5 | 2-trans,4-cis- | 3.06E−06 | 1.47 | 0.66 | Lipid metabolism pathway |
| | Decadienoyl- | | | | |
| | carnitine | | | | |
| 6 | 3-hydroxy- | 2.01E−06 | 1.29 | 0.62 | Lipid metabolism pathway |
| | dodecanoyl | | | | |
| | carnitine | | | | |
| 7 | Uridine | 2.46E−02 | 1.07 | 0.88 | Pyrimidine Metabolism |
| 8 | Diethylamine | 4.61E−03 | 1.05 | 0.99 | N/A |
| 9 | Pyruvic acid | 2.60E−02 | 1.12 | 1.24 | Citrate cycle (TCA cycle), |
| | | | | | Pyruvate metabolism |
| 10 | Methylaceto- | 3.27E−02 | 1.14 | 1.11 | N/A |
| | acetic acid | | | | |
| 11 | Asparagine | 8.53E−04 | 1.08 | 0.94 | Aspartate Metabolism, |
| | | | | | Ammonia Recycling |
| 12 | Urocanic acid | 6.75E−04 | 1.06 | 0.83 | Histidine Metabolism, |
| | | | | | Ammonia |
| | | | | | Recycling |
| 13 | N6,N6,N6- | 4.81E−06 | 1.40 | 0.68 | Carnitine Synthesis |
| | Tri- | | | | |
| | methylysine | | | | |
| 14 | Hippuric acid | 3.45E−04 | 1.68 | 0.40 | Phenylalanine metabolism |
| 15 | Hypoxanthine | 1.61E−03 | 1.44 | 1.24 | Purine Metabolism |
| 16 | Trimethyl- | 6.81E−04 | 1.09 | 0.54 | N/A |
| | amine | | | | |
| | N-oxide | | | | |
| 17 | 1-Methyl- | 2.43E−07 | 1.59 | 0.71 | Nicotinate and |
| | nicotinamide | | | | Nicotinamide |
| | | | | | Metabolism |
| 18 | Serotonin | 5.94E−05 | 1.23 | 0.77 | Tryptophan Metabolism |
| 19 | Lactic acid | 4.60E−03 | 1.40 | 1.19 | Gluconeogenesis, Pyruvate |
| | | | | | Metabolism |
| 20 | 3-hydroxy- | 2.54E−06 | 1.36 | 0.74 | Lipid metabolism pathway |
| | decanoyl | | | | |
| | carnitine | | | | |
| 21 | 3-hydroxy- | 8.46E−05 | 1.17 | 0.80 | Lipid metabolism pathway |
| | octanoyl | | | | |
| | carnitine | | | | |
| 22 | Hexanoyl- | 6.64E−04 | 1.08 | 0.70 | Lipid metabolism pathway |
| | carnitine | | | | |
| 23 | Octanoyl- | 5.05E−05 | 1.24 | 0.62 | Lipid metabolism pathway |
| | carnitine | | | | |
| 24 | Xanthine | 1.00E−03 | 1.51 | 1.23 | Purine Metabolism |
| 25 | Arabinosyl- | 4.59E−07 | 2.17 | 0.42 | N/A |
| | hypoxanthine | | | | |
| 26 | Inosine | 3.11E−08 | 1.51 | 0.42 | Purine Metabolism |
| 27 | Creatinine | 6.04E−04 | 1.15 | 0.96 | Arginine and proline |
| | | | | | metabolism |
| 28 | Ecgonine | 1.78E−04 | 1.20 | 0.74 | N/A |
| 29 | 4-oxo- | 2.04E−05 | 1.43 | 0.67 | Retinol Metabolism |
| | Retinoic acid | | | | |
| 30 | Acetophenone | 1.19E−03 | 1.58 | 0.64 | N/A |
| 31 | 7-Methyl- | 1.94E−04 | 1.27 | 0.92 | N/A |
| | guanine | | | | |
| 32 | 2-Pyrrolidone | 1.76E−08 | 1.70 | 1.32 | N/A |
| 33 | Succinic acid | 3.45E−04 | 1.63 | 1.47 | Alanine, aspartate and |
| | semialdehyde | | | | glutamate metabolism; |
| | | | | | Butanoate metabolism |
| 34 | Cyclohexane- | 3.46E−02 | 1.01 | 0.66 | N/A |
| | acetic acid | | | | |
| 35 | Ethyl | 6.11E−03 | 1.29 | 0.80 | N/A |
| | 3-oxo- | | | | |
| | hexanoate | | | | |
| 36 | 2-Ketobutyric | 4.77E−04 | 1.60 | 1.47 | Methionine Metabolism; |
| | acid | | | | Glycine and |
| | | | | | Serine Metabolism; |
| | | | | | Selenoamino Acid |
| | | | | | Metabolism |
| 37 | Homo-L- | 4.63E−04 | 1.13 | 0.78 | N/A |
| | arginine | | | | |
| 38 | 5-Oxoproline | 1.44E−05 | 1.72 | 1.21 | Glutathione metabolism |
| 39 | Citrulline | 1.92E−04 | 1.10 | 0.90 | Arginine and Proline |
| | | | | | Metabolism; Aspartate |
| | | | | | Metabolism; Urea Cycle |
| 40 | Pilocarpine | 5.52E−05 | 1.37 | 0.95 | N/A |
| 41 | Lysine | 2.83E−06 | 1.43 | 0.86 | Lysine Degradation; |
| | | | | | Biotin |
| | | | | | Metabolism |
| 42 | 3-Chloro- | 7.21E−03 | 1.33 | 0.38 | N/A |
| | tyrosine | | | | |
| 43 | Choline | 5.98E−05 | 1.16 | 0.77 | N/A |
| | Sulfate | | | | |

Note:
FC in the table was the multiple ratio of the lung cancer samples to the healthy samples in women;
and N/A indicated that no relevant metabolic pathway had been found

6. Different metabolites between group of patients with lung cancer and the group of patients with benign pulmonary nodules in women were shown in Table 7 below.

TABLE 7
Differential metabolites between the lung cancer samples and
the samples with benign lung nodules in women

| | | | | | Related metabolic |
|---|---|---|---|---|---|
| No. | Name | FDR | VIP | FC | pathway |
| 1 | Hippuric acid | 1.34E−03 | 1.75 | 0.51 | Phenylalanine metabolism |
| 2 | Hypoxanthine | 2.05E−02 | 1.51 | 1.22 | Purine Metabolism |
| 3 | Trimethylamine | 4.24E−02 | 1.05 | 0.85 | N/A |
| | N-oxide | | | | |
| 4 | 1-Methyl- | 1.36E−04 | 1.92 | 0.63 | Nicotinate and |
| | nicotinamide | | | | Nicotinamide |
| | | | | | Metabolism |
| 5 | Lactic acid | 1.22E−02 | 1.51 | 1.18 | Gluconeogenesis; Pyruvate |
| | | | | | Metabolism |
| 6 | Serotonin | 1.92E−04 | 1.89 | 0.72 | Tryptophan Metabolism |
| 7 | 3-hydroxy- | 2.80E−03 | 1.61 | 0.78 | Lipid metabolism pathway |
| | decanoyl | | | | |
| | carnitine | | | | |
| 8 | 3-hydroxy- | 6.31E−03 | 1.44 | 0.81 | Lipid metabolism pathway |
| | octanoyl | | | | |
| | carnitine | | | | |
| 9 | Hexanoyl- | 3.05E−02 | 1.13 | 0.82 | Lipid metabolism pathway |
| | carnitine | | | | |
| 10 | Octanoyl- | 6.01E−03 | 1.43 | 0.72 | Lipid metabolism pathway |
| | carnitine | | | | |
| 11 | Xanthine | 7.55E−03 | 1.69 | 1.23 | Purine Metabolism |
| 12 | Arabinosyl- | 1.91E−07 | 2.69 | 0.41 | N/A |
| | hypoxanthine | | | | |
| 13 | Inosine | 5.44E−08 | 2.34 | 0.41 | Purine Metabolism |
| 14 | Creatinine | 4.65E−02 | 1.24 | 0.95 | Arginine and proline |
| | | | | | metabolism |
| 15 | Ecgonine | 2.25E−03 | 1.47 | 0.72 | N/A |
| 16 | 4-oxo- | 2.22E−02 | 1.39 | 0.71 | Retinol Metabolism |
| | Retinoic acid | | | | |
| 17 | Acetophenone | 2.50E−02 | 1.22 | 0.56 | N/A |
| 18 | 7-Methyl- | 2.43E−02 | 1.26 | 0.92 | N/A |
| | guanine | | | | |
| 19 | 2-Pyrrolidone | 4.47E−02 | 1.08 | 1.13 | N/A |
| 20 | Succinic acid | 2.48E−02 | 1.31 | 1.28 | Alanine, aspartate and |
| | semialdehyde | | | | glutamate metabolism; |
| | | | | | Butanoate metabolism |
| 21 | Cyclohexane- | 2.87E−02 | 1.22 | 0.78 | N/A |
| | acetic acid | | | | |
| 22 | Ethyl 3- | 3.16E−02 | 1.15 | 0.81 | N/A |
| | oxohexanoate | | | | |
| 23 | 2-Ketobutyric | 3.16E−02 | 1.28 | 1.27 | Methionine Metabolism; |
| | acid | | | | Glycine and |
| | | | | | Serine Metabolism; |
| | | | | | Selenoamino Acid |
| | | | | | Metabolism |
| 24 | Homo-L- | 3.33E−04 | 1.68 | 0.78 | N/A |
| | arginine | | | | |
| 25 | 5-Oxoproline | 2.19E−06 | 2.24 | 1.24 | Glutathione metabolism |
| 26 | Citrulline | 2.07E−03 | 1.49 | 0.88 | Arginine and Proline |
| | | | | | Metabolism; Aspartate |
| | | | | | Metabolism; Urea Cycle |
| 27 | Lysine | 5.40E−03 | 1.31 | 0.90 | Lysine Degradation; |
| | | | | | Biotin Metabolism |
| 28 | 3- | 8.73E−03 | 1.59 | 0.51 | N/A |
| | Chlorotyrosine | | | | |
| 29 | Choline Sulfate | 1.11E−03 | 1.55 | 0.74 | N/A |
| 30 | Kynurenine | 5.73E−04 | 1.62 | 0.71 | Tryptophan Metabolism |
| 31 | 2-Octenoyl- | 2.47E−02 | 1.09 | 0.77 | Lipid metabolism pathway |
| | carnitine | | | | |
| 32 | cis-5-Tetra- | 1.39E−02 | 1.41 | 0.71 | Lipid metabolism pathway |
| | decenoyl- | | | | |
| | carnitine | | | | |
| 33 | Phenylalanine | 4.24E−02 | 1.14 | 1.15 | Phenylalanine, |
| | | | | | tyrosine and |
| | | | | | tryptophan biosynthesis; |
| | | | | | Phenylalanine metabolism |

Note:
FC in the table was the multiple ratio of the lung cancer samples to the samples with benign pulmonary nodules in men;
and N/A indicated that no relevant metabolic pathway had been found.

It could be seen from comparison of Tables 2 and 3 that:

(1) The following metabolites had significant differences between patients with lung cancer and patients with benign pulmonary nodules and between patients with lung cancer and healthy people: alpha-Eleostearic acid, 2-Ketobutyric acid, 2-Octenoylcarnitine, 2-trans, 4-cis-Decadienoylcarnitine, 3-Chlorotyrosine, 3-hydroxydecanoyl carnitine, 3-hydroxydodecanoyl carnitine, 3-hydroxyoctanoyl carnitine, Acetophenone, Arabinosylhypoxanthine, Cyclohexaneacetic acid, Dihydroxybenzoic acid, Docosahexaenoic acid, Ecgonine, Ethyl 3-oxohexanoate, Hexanoylcarnitine, Hippuric acid, Homo-L-arginine, Hypoxanthine, Lactic acid, N-Acetyl-L-alanine, Octanoylcarnitine, 5-Oxoproline, Pyruvic acid, Serotonin, Succinic acid semialdehyde, Xanthine;

(2) The following metabolites had significant differences between patients with lung cancer and patients with benign pulmonary nodules, but not between patients with lung cancer and healthy people: 1-Methylnicotinamide, 2-Pyrrolidone, 4-oxo-Retinoic acid, 7-Methylguanine, Acetylcarnitine, Bilirubin, Choline Sulfate, cis-5-Tetradecenoylcarnitine, Citrulline, Creatinine, Diethylamine, Dihydrothymine, Glutamine, Hydroxybutyric acid, Inosine, Kynurenine, Linoleyl carnitine, Lysine, Trimethylamine N-oxide.

It could be seen from comparison of Tables 4 and 5 that:

(1) The following metabolites had significant differences both between patients with lung cancer and patients with benign pulmonary nodules in men and between patients with lung cancer and healthy people in men: 1-Methylnicotinamide, 2-trans,4-cis-Decadienoylcarnitine, 3-hydroxydecanoyl carnitine, 3-hydroxydodecanoyl carnitine, 3-hydroxyoctanoyl carnitine, 4-oxo-Retinoic acid, 7-Methylguanine, Acetylcarnitine, alpha-Eleostearic acid, Arabinosylhypoxanthine, Cyclohexaneacetic acid, Diethylamine, Docosahexaenoic acid, Ecgonine, Ethyl 3-oxohexanoate, Glutamine, Hippuric acid, Homo-L-arginine, Hypoxanthine, Inosine, Linoleyl carnitine, N-Acetyl-L-alanine, Octanoylcarnitine, 5-Oxoproline, Pyruvic acid, Trimethylamine N-oxide;

(2) The following metabolites had significant differences between patients with lung cancer and patients with benign pulmonary nodules in men, but not between patients with lung cancer and healthy people in men: 2-Octenoylcarnitine, 3-hydroxybutyryl carnitine, Aminoadipic acid, Bilirubin, Dihydrothymine, Ergothioneine, Lactic acid, N6,N6,N6-Trimethylysine, Nicotine.

It could be seen from comparison of Tables 6 and 7 that:

(1) The following metabolites had significant differences both between patients with lung cancer and patients with benign pulmonary nodules in women and between patients with lung cancer and healthy people in women: 1-Methylnicotinamide, 2-Ketobutyric acid, 2-Pyrrolidone, 3-Chlorotyrosine, 3-hydroxydecanoyl carnitine, 3-hydroxyoctanoyl carnitine, 4-oxo-Retinoic acid, 7-Methylguanine, Acetophenone, Arabinosylhypoxanthine, Choline Sulfate, Citrulline, Creatinine, Cyclohexaneacetic acid, Ecgonine, Ethyl 3-oxohexanoate, Hexanoylcarnitine, Hippuric acid, Homo-L-arginine, Hypoxanthine, Inosine, Lactic acid, Lysine, Octanoylcarnitine, 5-Oxoproline, Serotonin, Succinic acid semialdehyde, Trimethylamine N-oxide, Xanthine;

(2) The following metabolites had significant differences between patients with lung cancer and patients with benign pulmonary nodules in women, but not between patients with lung cancer and healthy people in women: 2-Octenoylcarnitine, cis-5-Tetradecenoylcarnitine, Kynurenine, Phenylalanine.

It could be seen from comparison of Tables 3, 5 and 7 that:

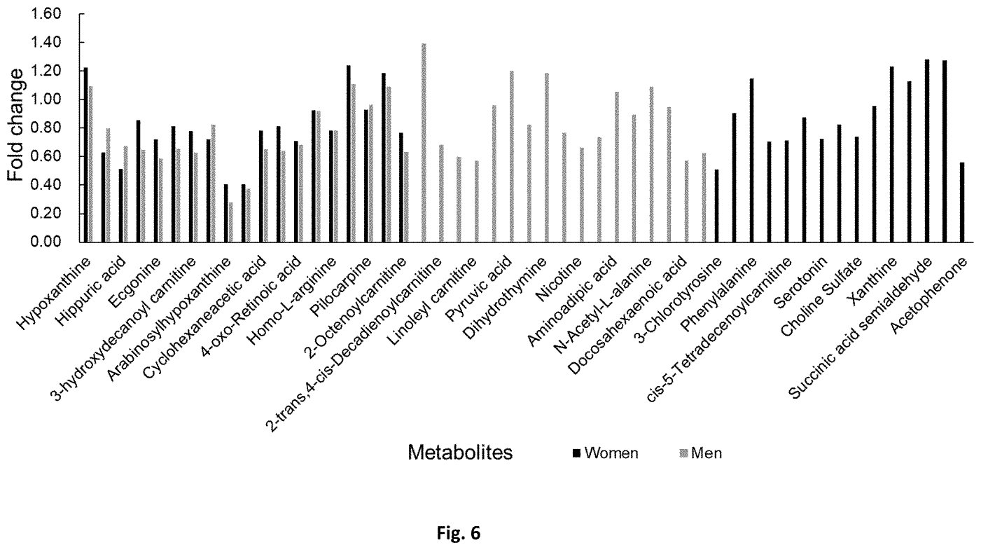

there were both common ones and different ones of the differential metabolites between patients with lung cancer and patients with benign pulmonary nodules in men and women. The differential metabolite that was common in the patients with lung cancer and the patients with benign pulmonary nodules in men and women, and differential metabolites specific to the patients with lung cancer and the patients with benign pulmonary nodules in men or women were shown in FIG. 6, wherein:

(1) The metabolite that was common between patients with lung cancer and patients with benign pulmonary nodules in men and women included: 1-Methylnicotinamide, 2-Octenoylcarnitine, 3-hydroxydecanoyl carnitine, 3-hydroxyoctanoyl carnitine, 4-oxo-Retinoic acid, 7-Methylguanine, Arabinosylhypoxanthine, Cyclohexaneacetic acid, Ecgonine, Ethyl 3-oxohexanoate, Hippuric acid, Homo-L-arginine, Hypoxanthine, Inosine, Lactic acid, Octanoylcarnitine, 5-Oxoproline, Trimethylamine N-oxide;

(2) The metabolite that had significant differences between patients with lung cancer and patients with benign pulmonary nodules in men, but not in women, included: alpha-Eleostearic acid, 2-trans,4-cis-Decadienoylcarnitine, 3-hydroxydodecanoyl carnitine, Acetylcarnitine, Bilirubin, Diethylamine, Dihydrothymine, Docosahexaenoic acid, Glutamine, Linoleyl carnitine, N-Acetyl-L-alanine, Pyruvic acid, 3-hydroxybutyryl carnitine, Aminoadipic acid, Ergothioneine, N6,N6,N6-Trimethylysine, Nicotine;

(3) The metabolite that had significant differences between patients with lung cancer and patients with benign pulmonary nodules in women, but not in men, included: 2-Ketobutyric acid, 2-Pyrrolidone, 3-Chlorotyrosine, Acetophenone, Choline Sulfate, cis-5-Tetradecenoylcarnitine, Citrulline, Creatinine, Hexanoylcarnitine, Kynurenine, Lysine, Serotonin, Succinic acid semialdehyde, Xanthine, Phenylalanine.

It could be seen from comparison of Tables 2 to 7 that:

(1) the specific metabolite between patients with lung cancer and healthy people in men included: 3b,16a-Dihydroxyandrostenone sulfate, Isoleucine, Leucine, Tyrosine;

(2) the specific metabolite between patients with lung cancer and patients with benign pulmonary nodules in men included: 3-hydroxybutyryl carnitine, Aminoadipic acid, Ergothioneine, Nicotine;

(3) the specific metabolite between patients with lung cancer and healthy people in women included: Alanine, Asparagine, Propionylcarnitine, Urocanic acid;

(4) the specific metabolite between patients with lung cancer and patients with benign pulmonary nodules in women included: Phenylalanine. Here, the specific differential metabolite referred to that these differential metabolites only had significant differences between two specific groups, but not between other groups.

### EXAMPLE 5

### Model for Differential Diagnosis of Lung Cancer and Benign Pulmonary Nodules and Establishment Thereof

1. The model for differential diagnosis of lung cancer and benign pulmonary nodules by a single differential metabolite and establishment thereof

An ROC curve of each metabolite was established, and the quality of the experimental results was judged by the area under the curve (AUC). The AUC of 0.5 indicated that the single metabolite had no diagnostic value; the AUC greater than 0.5 indicated that the single metabolite had a diagnostic value; and the diagnostic value of the single metabolite was higher when the AUC was larger.

The respective metabolites in Tables 3, 5 and 7 were analyzed by ROC curve, and the ROC values and related information of each metabolite were shown in Tables 8, 9 and 10, respectively:

TABLE 8
ROC values and related information of differential metabolites between lung cancer
samples and samples with benign pulmonary nodules as obtained by ROC Analysis

| | | | 95% confidence | | | Cut-off |
|---|---|---|---|---|---|---|
| No. | Metabolites | AUC | interval | Sensitivity | Specificity | value |
| V01 | 2-Ketobutyric acid | 0.568 | 0.508-0.642 | 0.6 | 0.5 | 1.130 |
| V02 | Succinic acid semialdehyde | 0.568 | 0.506-0.632 | 0.6 | 0.5 | 1.210 |
| V03 | Acetophenone | 0.617 | 0.546-0.677 | 0.7 | 0.6 | 0.993 |
| V04 | 5-Oxoproline | 0.736 | 0.682-0.786 | 0.6 | 0.7 | 1.030 |
| V05 | N-Acetyl-L-alanine | 0.561 | 0.487-0.622 | 0.7 | 0.5 | 1.040 |
| V06 | Hypoxanthine | 0.598 | 0.534-0.662 | 0.6 | 0.5 | 0.970 |
| V07 | Cyclohexaneacetic acid | 0.676 | 0.617-0.736 | 0.7 | 0.6 | 0.971 |
| V08 | Xanthine | 0.558 | 0.401-0.627 | 0.4 | 0.7 | 1.140 |
| V09 | Dihydroxybenzoic acid | 0.640 | 0.573-0.695 | 0.6 | 0.6 | 0.666 |
| V10 | Ethyl 3-oxohexanoate | 0.671 | 0.612-0.738 | 0.6 | 0.7 | 0.764 |
| V11 | Hippuric acid | 0.665 | 0.606-0.727 | 0.6 | 0.7 | 0.327 |
| V12 | 3-Chlorotyrosine | 0.636 | 0.584-0.699 | 0.6 | 0.6 | 0.417 |
| V13 | Arabinosylhypoxanthine | 0.784 | 0.731-0.836 | 0.8 | 0.7 | 0.505 |
| V14 | Docosahexaenoic acid | 0.683 | 0.621-0.738 | 0.7 | 0.7 | 0.952 |
| V15 | Pyruvic acid | 0.578 | 0.514-0.637 | 0.6 | 0.6 | 1.220 |
| V16 | Lactic acid | 0.599 | 0.534-0.667 | 0.6 | 0.5 | 1.010 |
| V17 | Hydroxybutyric acid | 0.585 | 0.521-0.644 | 0.6 | 0.6 | 1.370 |
| V18 | Dihydrothymine | 0.585 | 0.525-0.649 | 0.6 | 0.5 | 0.780 |
| V19 | Serotonin | 0.641 | 0.583-0.704 | 0.6 | 0.6 | 0.933 |
| V20 | Ecgonine | 0.710 | 0.651-0.769 | 0.7 | 0.7 | 0.783 |
| V21 | Homo-L-arginine | 0.654 | 0.591-0.715 | 0.6 | 0.6 | 0.856 |
| V22 | Hexanoylcarnitine | 0.604 | 0.545-0.674 | 0.7 | 0.5 | 0.997 |
| V23 | alpha-Eleostearic acid | 0.667 | 0.605-0.723 | 0.6 | 0.7 | 0.843 |
| V24 | 2-Octenoylcarnitine | 0.651 | 0.594-0.708 | 0.7 | 0.6 | 1.040 |
| V25 | Octanoylcarnitine | 0.656 | 0.594-0.718 | 0.7 | 0.6 | 0.856 |
| V26 | 3-hydroxyoctanoylcarnitine | 0.682 | 0.622-0.741 | 0.6 | 0.6 | 0.887 |
| V27 | 2-trans,4-cis-Decadienoylcarnitine | 0.637 | 0.570-0.694 | 0.5 | 0.7 | 0.761 |
| V28 | 3-hydroxydecanoyl carnitine | 0.690 | 0.631-0.745 | 0.6 | 0.7 | 0.897 |
| V29 | 3-hydroxydodecanoylcarnitine | 0.658 | 0.593-0.719 | 0.6 | 0.7 | 0.793 |
| V30 | Creatinine | 0.550 | 0.485-0.609 | 0.5 | 0.6 | 0.986 |
| V31 | 1-Methylnicotinamide | 0.660 | 0.598-0.724 | 0.5 | 0.7 | 0.839 |
| V32 | Glutamine | 0.592 | 0.523-0.653 | 0.5 | 0.7 | 1.040 |
| V33 | Lysine | 0.583 | 0.517-0.644 | 0.6 | 0.6 | 0.939 |
| V34 | 7-Methylguanine | 0.614 | 0.550-0.676 | 0.6 | 0.6 | 1.130 |
| V35 | Citrulline | 0.568 | 0.504-0.629 | 0.5 | 0.7 | 0.994 |
| V36 | Choline Sulfate | 0.661 | 0.593-0.720 | 0.7 | 0.6 | 0.739 |
| V37 | Acetyl carnitine | 0.595 | 0.525-0.661 | 0.5 | 0.6 | 0.981 |
| V38 | Kynurenine | 0.616 | 0.553-0.675 | 0.7 | 0.5 | 1.330 |
| V39 | Inosine | 0.747 | 0.684-0.798 | 0.7 | 0.7 | 0.400 |
| V40 | 4-oxo-Retinoic acid | 0.621 | 0.557-0.684 | 0.7 | 0.5 | 1.090 |
| V41 | cis-5-Tetradecenoylcarnitine | 0.579 | 0.522-0.643 | 0.5 | 0.6 | 0.906 |
| V42 | Linoleylcarnitine | 0.649 | 0.582-0.707 | 0.6 | 0.7 | 1.110 |
| V43 | Bilirubin | 0.603 | 0.538-0.664 | 0.6 | 0.6 | 1.110 |
| V44 | Diethylamine | 0.650 | 0.584-0.713 | 0.6 | 0.7 | 1.010 |
| V45 | Trimethylamine N-oxide | 0.643 | 0.576-0.697 | 0.6 | 0.6 | 0.520 |
| V46 | 2-Pyrrolidone | 0.643 | 0.583-0.699 | 0.6 | 0.7 | 1.320 |

TABLE 9
ROC values and related information of differential metabolites between lung cancer samples
and samples with benign pulmonary nodules in men as obtained by ROC Analysis

| | | | 95% confidence | | | Cut-off |
|---|---|---|---|---|---|---|
| No. | Metabolites | AUC | interval | Sensitivity | Specificity | value |
| MV01 | Dihydrothymine | 0.619 | 0.534-0.703 | 0.7 | 0.6 | 1.160 |
| MV02 | 5-Oxoproline | 0.643 | 0.558-0.734 | 0.6 | 0.6 | 1.030 |
| MV03 | N-Acetyl-L-alanine | 0.598 | 0.494-0.684 | 0.5 | 0.6 | 1.140 |
| MV04 | Hypoxanthine | 0.559 | 0.470-0.650 | 0.6 | 0.5 | 0.997 |
| MV05 | 1-Methylnicotinamide | 0.641 | 0.549-0.726 | 0.5 | 0.7 | 0.839 |
| MV06 | Cyclohexaneacetic acid | 0.725 | 0.633-0.806 | 0.7 | 0.6 | 1.110 |
| MV07 | Glutamine | 0.581 | 0.492-0.674 | 0.5 | 0.6 | 1.050 |
| MV08 | Ethyl 3-oxohexanoate | 0.752 | 0.670-0.829 | 0.6 | 0.8 | 0.792 |
| MV09 | Aminoadipic acid | 0.564 | 0.472-0.659 | 0.6 | 0.6 | 1.180 |
| MV10 | Nicotine | 0.672 | 0.584-0.750 | 0.7 | 0.6 | 0.950 |
| MV11 | 7-Methylguanine | 0.623 | 0.535-0.712 | 0.6 | 0.6 | 1.210 |
| MV12 | Hippuric acid | 0.655 | 0.571-0.750 | 0.7 | 0.5 | 0.492 |
| MV13 | Ecgonine | 0.774 | 0.699-0.843 | 0.8 | 0.7 | 0.781 |
| MV14 | Homo-L-arginine | 0.650 | 0.562-0.730 | 0.6 | 0.7 | 0.856 |
| MV15 | N6,N6,N6-Trimethylysine | 0.702 | 0.617-0.782 | 0.7 | 0.7 | 1.040 |
| MV16 | Acetylcarnitine | 0.662 | 0.572-0.743 | 0.5 | 0.7 | 0.911 |
| MV17 | Ergothioneine | 0.648 | 0.550-0.731 | 0.5 | 0.7 | 0.622 |
| MV18 | 3-hydroxybutyryl carnitine | 0.649 | 0.570-0.730 | 0.7 | 0.6 | 0.872 |
| MV19 | Arabinosylhypoxanthine | 0.784 | 0.713-0.848 | 0.8 | 0.7 | 0.442 |
| MV20 | Inosine | 0.716 | 0.631-0.801 | 0.7 | 0.7 | 0.325 |
| MV21 | alpha-Eleostearic acid | 0.757 | 0.666-0.832 | 0.7 | 0.7 | 0.868 |
| MV22 | 2-Octenoylcarnitine | 0.687 | 0.599-0.773 | 0.7 | 0.6 | 1.050 |
| MV23 | Octanoylcarnitine | 0.660 | 0.572-0.752 | 0.6 | 0.7 | 0.802 |
| MV24 | 3-hydroxyoctanoyl carnitine | 0.738 | 0.656-0.808 | 0.7 | 0.7 | 0.994 |
| MV25 | 2-trans,4-cis-Decadienoylcarnitine | 0.720 | 0.633-0.799 | 0.7 | 0.7 | 0.924 |
| MV26 | 4-oxo-Retinoic acid | 0.639 | 0.553-0.716 | 0.4 | 0.8 | 0.730 |
| MV27 | Docosahexaenoic acid | 0.762 | 0.688-0.835 | 0.7 | 0.7 | 0.932 |
| MV28 | 3-hydroxydecanoyl carnitine | 0.728 | 0.639-0.797 | 0.6 | 0.7 | 0.975 |
| MV29 | 3-hydroxydodecanoyl carnitine | 0.712 | 0.618-0.790 | 0.7 | 0.7 | 1.090 |
| MV30 | Linoleyl carnitine | 0.867 | 0.807-0.922 | 0.8 | 0.9 | 1.210 |
| MV31 | Bilirubin | 0.651 | 0.562-0.736 | 0.6 | 0.7 | 1.070 |
| MV32 | Diethylamine | 0.663 | 0.570-0.759 | 0.6 | 0.8 | 0.986 |
| MV33 | Trimethylamine N-oxide | 0.683 | 0.608-0.771 | 0.6 | 0.7 | 0.521 |
| MV34 | Pyruvic acid | 0.602 | 0.509-0.690 | 0.7 | 0.5 | 1.240 |
| MV35 | Lactic acid | 0.569 | 0.477-0.657 | 0.6 | 0.6 | 1.120 |

TABLE 10
ROC values and related information of differential metabolites between lung cancer samples
and samples with benign pulmonary nodules in women as obtained by ROC Analysis

| | | | 95% confidence | | | Cut-off |
|---|---|---|---|---|---|---|
| No. | Metabolites | AUC | interval | Sensitivity | Specificity | value |
| FV01 | 2-Ketobutyric acid | 0.614 | 0.528-0.705 | 0.6 | 0.5 | 1.070 |
| FV02 | Succinic acid semialdehyde | 0.622 | 0.529-0.704 | 0.7 | 0.5 | 1.060 |
| FV03 | Creatinine | 0.604 | 0.520-0.693 | 0.4 | 0.8 | 0.861 |
| FV04 | Acetophenone | 0.630 | 0.541-0.720 | 0.8 | 0.5 | 0.899 |
| FV05 | 5-Oxoproline | 0.823 | 0.748-0.887 | 0.8 | 0.7 | 0.987 |
| FV06 | Hypoxanthine | 0.646 | 0.558-0.727 | 0.6 | 0.6 | 0.970 |
| FV07 | 1-Methylnicotinamide | 0.679 | 0.592-0.758 | 0.6 | 0.7 | 0.931 |
| FV08 | Cyclohexaneacetic acid | 0.630 | 0.539-0.721 | 0.6 | 0.6 | 0.849 |
| FV09 | Lysine | 0.630 | 0.547-0.725 | 0.7 | 0.6 | 0.895 |
| FV10 | Xanthine | 0.649 | 0.565-0.737 | 0.5 | 0.8 | 1.130 |
| FV11 | Ethyl 3-oxohexanoate | 0.616 | 0.513-0.702 | 0.5 | 0.7 | 0.632 |
| FV12 | 7-Methylguanine | 0.606 | 0.522-0.685 | 0.7 | 0.6 | 1.110 |
| FV13 | Phenylalanine | 0.702 | 0.610-0.790 | 0.7 | 0.6 | 1.080 |
| FV14 | Citrulline | 0.642 | 0.547-0.720 | 0.6 | 0.6 | 0.994 |
| FV15 | Serotonin | 0.698 | 0.612-0.780 | 0.7 | 0.6 | 0.893 |
| FV16 | Hippuric acid | 0.687 | 0.594-0.761 | 0.7 | 0.7 | 0.327 |
| FV17 | Choline Sulfate | 0.674 | 0.594-0.758 | 0.7 | 0.6 | 0.713 |
| FV18 | Ecgonine | 0.656 | 0.559-0.742 | 0.7 | 0.6 | 0.915 |
| FV19 | Homo-L-arginine | 0.678 | 0.586-0.762 | 0.6 | 0.7 | 0.750 |
| FV20 | Kynurenine | 0.660 | 0.567-0.746 | 0.8 | 0.5 | 1.310 |
| FV21 | 3-Chlorotyrosine | 0.653 | 0.566-0.740 | 0.6 | 0.7 | 0.297 |
| FV22 | Hexanoylcarnitine | 0.595 | 0.507-0.682 | 0.7 | 0.5 | 0.997 |
| FV23 | Arabinosylhypoxanthine | 0.798 | 0.724-0.865 | 0.7 | 0.8 | 0.540 |
| FV24 | Inosine | 0.787 | 0.708-0.861 | 0.7 | 0.8 | 0.402 |
| FV25 | 2-Octenoylcarnitine | 0.618 | 0.534-0.708 | 0.6 | 0.7 | 0.794 |
| FV26 | Octanoylcarnitine | 0.641 | 0.542-0.724 | 0.7 | 0.6 | 0.867 |
| FV27 | 3-hydroxyoctanoylcarnitine | 0.632 | 0.551-0.720 | 0.6 | 0.6 | 0.858 |
| FV28 | 4-oxo-Retinoic acid | 0.607 | 0.507-0.687 | 0.6 | 0.5 | 1.090 |
| FV29 | 3-hydroxydecanoylcarnitine | 0.654 | 0.564-0.737 | 0.6 | 0.6 | 0.798 |
| FV30 | cis-5-Tetradecenoylcarnitine | 0.615 | 0.534-0.708 | 0.5 | 0.6 | 0.910 |
| FV31 | Trimethylamine N-oxide | 0.599 | 0.505-0.690 | 0.5 | 0.7 | 0.389 |
| FV32 | 2-Pyrrolidone | 0.611 | 0.523-0.712 | 0.6 | 0.6 | 1.160 |
| FV33 | Lactic acid | 0.629 | 0.526-0.719 | 0.6 | 0.6 | 1.020 |

2. The model for differential diagnosis of lung cancer and benign pulmonary nodules by a combination of multiple differential metabolites and establishment thereof

Based on the relative abundance of different metabolites between lung cancer and pulmonary nodules in Table 3, a model for differential diagnosis of lung cancer and benign pulmonary nodules was established by using binary logistic regression (SPSS software), and the forward maximum likelihood method (LR) was adopted to screen the optimum model parameters (SPSS software) for differential diagnosis of lung cancer and pulmonary nodules. As a result, a prediction model A (applicable to both men and women) was obtained.

The odds ratio (OR) referred to the ratio of occurrence and non-occurrence of lung cancer, which was an indicator of the correlation strength between lung cancer and a predictive variable. OR>1 indicated that with the increase of this variable, the probability of occurrence of lung cancer was increased, which was a “positive” correlation; OR<1 indicated that with the increase of this variable, the probability of occurrence of lung cancer was decreased, which was a “negative” correlation; and OR=1 indicated that there was no correlation between the disease and exposure. In logistic regression, the coefficient obtained by us was the logarithm of the OR value. p<0.05 in the table showed that this variable played a significant role in the model.

The variables and parameters of model A were listed in Table 11 below:

TABLE 11
List of variables and parameters of model A

| | | Model | | | Odds |
|---|---|---|---|---|---|
| | | co- | Standard | Significant | ratio |
| No. | Model variable | efficient | error | p | (OR) |
| / | Constant | 1.610 | 1.874 | 0.390 | / |
| V04 | 5-Oxoproline | 5.553 | 1.364 | 4.70E−05 | 258.105 |
| V05 | N-Acetyl- | 2.920 | 1.133 | 0.010 | 18.544 |
| | L-alanine | | | | |
| V06 | Hypoxanthine | 2.713 | 0.806 | 0.001 | 15.080 |
| V07 | Cyclohexane- | −0.332 | 0.134 | 0.013 | 0.718 |
| | acetic acid | | | | |
| V10 | Ethyl | −1.798 | 0.456 | 8.00E−05 | 0.166 |
| | 3-oxohexanoate | | | | |
| V13 | Arabinosyl- | −7.922 | 1.881 | 2.50E−05 | 3.63E−04 |
| | hypoxanthine | | | | |
| V14 | Docosahexaenoic | −0.593 | 0.204 | 0.004 | 0.553 |
| | acid | | | | |
| V17 | Hydroxybutyric | 0.643 | 0.270 | 0.017 | 1.902 |
| | acid | | | | |
| V19 | Serotonin | −2.187 | 0.537 | 0.000 | 0.112 |
| V20 | Ecgonine | −0.992 | 0.425 | 0.019 | 0.371 |
| V33 | Lysine | −2.352 | 0.980 | 0.016 | 0.095 |
| V38 | Kynurenine | −1.441 | 0.380 | 1.50E−04 | 0.237 |
| V39 | Inosine | 7.214 | 1.978 | 2.65E−04 | 1357.885 |
| V40 | 4-oxo- | −1.220 | 0.410 | 0.003 | 0.295 |
| | Retinoic acid | | | | |
| V42 | Linoleylcarnitine | −1.235 | 0.334 | 2.19E−04 | 0.291 |

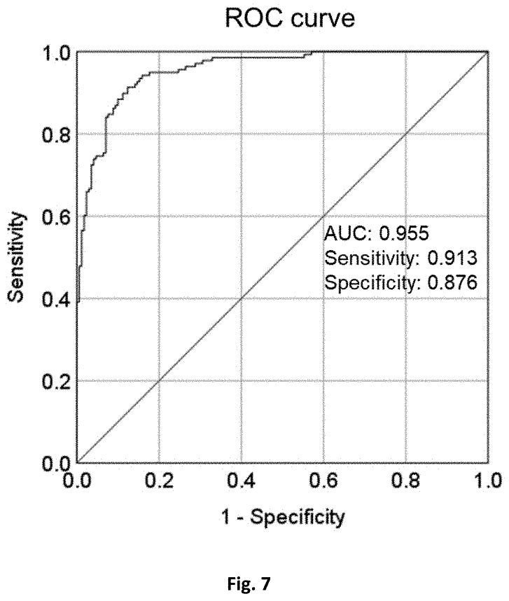

Finally, the resultant equation of model A was: Logit(P)=In[P/((1−P)]=5.553×V04+2.92×V05+2.713×V06−0.332×V07−1.798×V10−7.922×V13−0.593×V14+0.643×V17−2.187×V19−0.992×V20−2.352×V33−1.441×V38+7.214×V39−1.22×V40−1.235×V42+1.61, and the cut-off value of P was 0.424 (that is, when P>0.424, lung cancer was diagnosed). As shown in FIG. 7, ROC analysis was conducted, the AUC was 0.955, and the sensitivity and specificity were 0.913 and 0.876, respectively, indicating that the model A could be used for differential diagnosis of benign pulmonary nodules and malignant tumors.

Further, considering the gender factor, a model B for differential diagnosis of lung cancer and pulmonary nodules in men and a model C for differential diagnosis of lung cancer and benign pulmonary nodules in women were established according to Tables 5 and 7, respectively.

The variables and parameters of model B were listed in Table 12 below:

TABLE 12
List of variables and parameters of model B (men)

| | | | | | Odds |
|---|---|---|---|---|---|
| | Model | Model | Standard | Significant | ratio |
| No. | variable | coefficient | error | p | (OR) |
| / | Constant | 1.494 | 2.090 | 0.475 | / |
| MV02 | 5-Oxoproline | 6.283 | 1.945 | 0.001 | 535.214 |
| MV10 | Nicotine | −0.646 | 0.255 | 0.011 | 0.524 |
| MV13 | Ecgonine | −2.758 | 0.800 | 0.001 | 0.063 |
| MV15 | N6,N6,N6- | 1.864 | 0.822 | 0.023 | 6.453 |
| | Trimethylysine | | | | |
| MV19 | Arabinosyl- | −1.126 | 0.582 | 0.053 | 0.324 |
| | hypoxanthine | | | | |
| MV27 | Docosa- | −1.145 | 0.563 | 0.042 | 0.318 |
| | hexaenoic acid | | | | |
| MV30 | Linoleyl | −3.918 | 0.844 | 3.48E−06 | 0.020 |
| | carnitine | | | | |

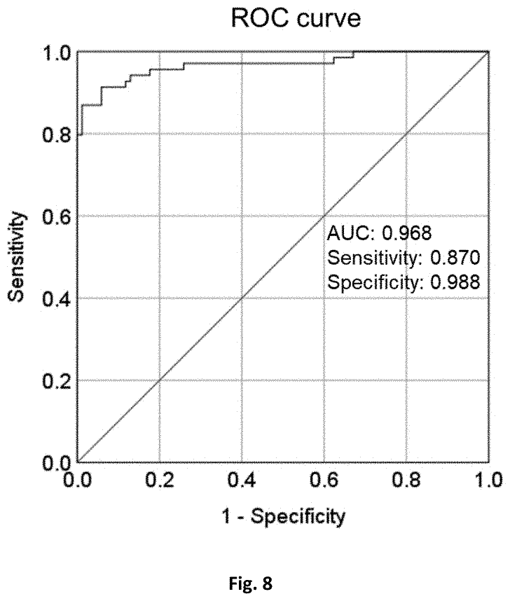

The equation of model B was: Logit(P)=In[P/((1−P)]=6.283×MV02−0.646×MV10−2.758×MV13+1.864×MV15−1.126×MV19−1.145×MV27−3.918×MV30+1.494, wherein the cut-off value of P was 0.701, and when P>0.701, it indicated that a man with nodules was a patient with lung cancer. As shown in FIG. 8, ROC analysis was conducted, the AUC was 0.968, and the sensitivity and specificity were 0.870 and 0.988, respectively, indicating that the model B could be used for differential diagnosis of benign pulmonary nodules and malignant tumors in men.

The variables and parameters of model C were listed in Table 13 below:

TABLE 13
List of variables and parameters of model C (women)

| | Model | Model | Standard | Significant | Odds ratio |
|---|---|---|---|---|---|
| No. | variable | coefficient | error | p | (OR) |
| / | Constant | −6.905 | 3.466 | 0.046 | / |
| FV05 | 5-Oxoproline | 10.742 | 2.599 | 3.57E−05 | 46244.867 |
| FV08 | Cyclo- | −1.031 | 0.398 | 0.010 | 0.357 |
| | hexaneacetic | | | | |
| | acid | | | | |
| FV09 | Lysine | −7.442 | 2.061 | 3.04E-04 | 0.001 |
| FV13 | Phenylalanine | 11.839 | 2.959 | 6.31E−05 | 138602.684 |
| FV15 | Serotonin | −2.617 | 0.994 | 0.008 | 0.073 |
| FV20 | Kynurenine | −3.030 | 0.801 | 1.54E−04 | 0.048 |
| FV23 | Arabinosyl- | −1.413 | 0.593 | 0.017 | 0.243 |
| | hypoxanthine | | | | |
| FV29 | 3-hydroxy- | −2.278 | 0.805 | 0.005 | 0.103 |
| | decanoyl | | | | |
| | carnitine | | | | |

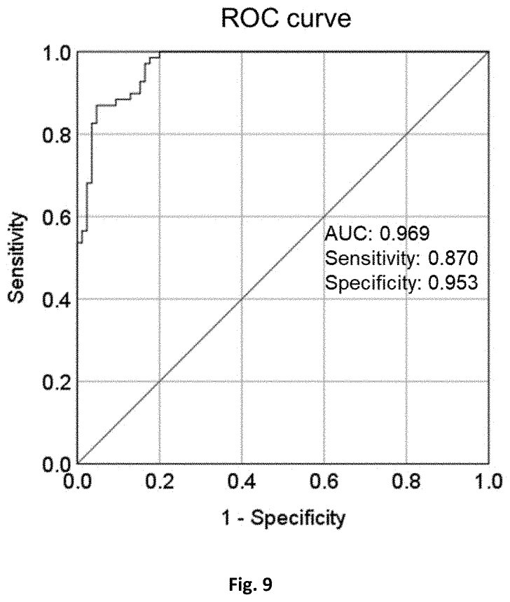

The equation of model C was: Logit(P)=In[P/((1−P)]=10.742×FV05−1.031×FV08−7.442×FV09+11.839×FV13−2.617×FV15−3.030×FV20−1.413×FV23−2.278×FV29−6.905, wherein the cut-off value of P was 0.629, and when P>0.629, it indicated that a woman with nodules was a patient with lung cancer. As shown in FIG. 9, ROC analysis was conducted, the AUC was 0.969, and the sensitivity and specificity were 0.870 and 0.953, respectively, indicating that the model C could be used for differential diagnosis of benign pulmonary nodules and malignant tumors in women.

3. The model for differential diagnosis of lung cancer and benign pulmonary nodules by a combination of all differential metabolites and establishment thereof

Based on the relative abundance of the differential metabolite in lung cancer and pulmonary nodules from Table 3, a model D for differential diagnosis with all differential metabolites between lung cancer and benign pulmonary nodules was established by using binary logistic regression (MetaboAnalyst software), and 10-fold Cross-Validation was adopted. The variables and parameters of model D were listed in Table 14 below:

TABLE 14
List of variables and parameters of model D

| | | | | | Odds |
|---|---|---|---|---|---|
| | | Model | Standard | Significant | ratio |
| No. | Model variable | coefficient | error | p | (OR) |
| / | Constant | 7.810 | 4.974 | 0.116 | / |
| V01 | 2-Ketobutyric acid | −17.026 | 7.874 | 0.031 | 4.03E−08 |
| V02 | Succinic acid | 17.418 | 8.036 | 0.030 | 3.67E+07 |
| | semialdehyde | | | | |
| V03 | Acetophenone | 0.200 | 0.252 | 0.428 | 1.220 |
| V04 | 5-Oxoproline | 6.450 | 1.992 | 0.001 | 632.780 |
| V05 | N-Acetyl-L-alanine | 1.479 | 1.732 | 0.393 | 4.390 |
| V06 | Hypoxanthine | 3.762 | 1.445 | 0.009 | 43.040 |
| V07 | Cyclohexane- | −0.337 | 0.198 | 0.088 | 0.710 |
| | acetic acid | | | | |
| V08 | Xanthine | −0.096 | 1.072 | 0.929 | 0.910 |
| V09 | Dihydroxy- | −0.681 | 0.372 | 0.067 | 0.510 |
| | benzoic acid | | | | |
| V10 | Ethyl | −2.144 | 0.758 | 0.005 | 0.120 |
| | 3-oxohexanoate | | | | |
| V11 | Hippuric acid | 0.654 | 1.684 | 0.698 | 1.920 |
| V12 | 3-Chlorotyrosine | −0.833 | 1.558 | 0.593 | 0.430 |
| V13 | Arabinosyl- | −10.388 | 3.203 | 0.001 | 3.08E−05 |
| | hypoxanthine | | | | |
| V14 | Docosahexaenoic | −1.051 | 0.340 | 0.002 | 0.350 |
| | acid | | | | |
| V15 | Pyruvic acid | −1.526 | 1.180 | 0.196 | 0.220 |
| V16 | Lactic acid | 1.505 | 1.942 | 0.438 | 4.500 |
| V17 | Hydroxybutyric | 1.806 | 1.027 | 0.079 | 6.090 |
| | acid | | | | |
| V18 | Dihydrothymine | 0.519 | 0.454 | 0.253 | 1.680 |
| V19 | Serotonin | −2.051 | 0.663 | 0.002 | 0.130 |
| V20 | Ecgonine | −0.860 | 0.670 | 0.199 | 0.420 |
| V21 | Homo-L-arginine | −0.552 | 0.783 | 0.481 | 0.580 |
| V22 | Hexanoylcarnitine | −3.683 | 2.311 | 0.111 | 0.030 |
| V23 | alpha-Eleostearic | 0.091 | 0.353 | 0.797 | 1.100 |
| | acid | | | | |
| V24 | 2-Octenoylcarnitine | −0.721 | 0.549 | 0.189 | 0.490 |
| V25 | Octanoylcarnitine | 1.430 | 1.629 | 0.380 | 4.180 |
| V26 | 3-hydroxy- | 0.572 | 2.242 | 0.799 | 1.770 |
| | octanoylcarnitine | | | | |
| V27 | 2-trans,4-cis- | 1.466 | 0.990 | 0.139 | 4.330 |
| | Decadienoyl- | | | | |
| | carnitine | | | | |
| V28 | 3-hydroxy- | −1.097 | 2.424 | 0.651 | 0.330 |
| | decanoylcarnitine | | | | |
| V29 | 3-hydroxy- | 0.272 | 0.948 | 0.774 | 1.310 |
| | dodecanoyl- | | | | |
| | carnitine | | | | |
| V30 | Creatinine | −0.315 | 2.478 | 0.899 | 0.730 |
| V31 | 1-Methyl- | −1.120 | 0.488 | 0.022 | 0.330 |
| | nicotinamide | | | | |
| V32 | Glutamine | −2.830 | 2.480 | 0.254 | 0.060 |
| V33 | Lysine | −2.850 | 1.577 | 0.071 | 0.060 |
| V34 | 7-Methylguanine | 0.993 | 1.908 | 0.603 | 2.700 |
| V35 | Citrulline | 2.321 | 1.557 | 0.136 | 10.190 |
| V36 | Choline Sulfate | −0.710 | 0.269 | 0.008 | 0.490 |
| V37 | Acetylcarnitine | −0.616 | 1.516 | 0.685 | 0.540 |
| V38 | Kynurenine | −1.711 | 0.573 | 0.003 | 0.180 |
| V39 | Inosine | 9.051 | 3.429 | 0.008 | 8530.730 |
| V40 | 4-oxo-Retinoic acid | −1.520 | 0.527 | 0.004 | 0.220 |
| V41 | cis-5-Tetra- | 0.302 | 0.773 | 0.696 | 1.350 |
| | decenoylcarnitine | | | | |
| V42 | Linoleylcarnitine | −1.688 | 0.514 | 0.001 | 0.180 |
| V43 | Bilirubin | −0.739 | 0.585 | 0.206 | 0.480 |
| V44 | Diethylamine | −0.152 | 3.060 | 0.960 | 0.860 |
| V45 | Trimethylamine | −0.282 | 0.591 | 0.634 | 0.750 |
| | N-oxide | | | | |
| V46 | 2-Pyrrolidone | −0.085 | 0.739 | 0.909 | 0.920 |

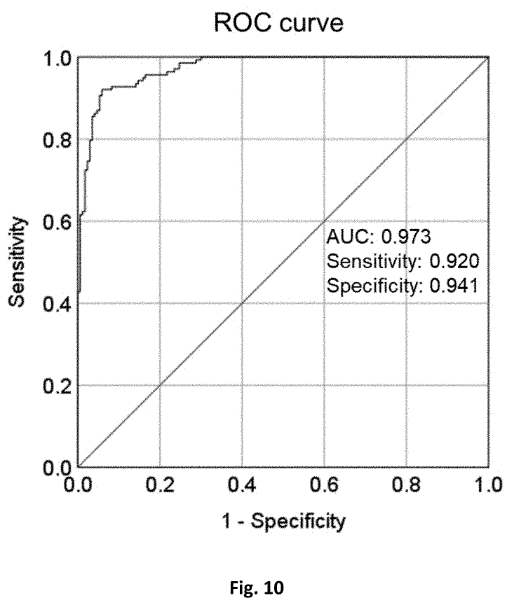

The equation of the model D was: Logit(P)=In[PA(1−P)]=−17.026×V01+17.418×V02+0.2×V03+6.45×V04+1.479×V05+3.762×V06−0.337×V07−0.096×V08−0.681×V09−2.144×V10+0.654×V11−0.833×V12−10.388×V13−1.051×V14−1.526×V15+1.505×V16+1.806×V17+0.519×V18−2.051×V19−0.86×V20−0.552×V21−3.683×V22+0.091×V23−0.721×V24+1.43×V25+0.572×V26+1.466×V27−1.097×V28+0.272×V29−0.315×V30−1.12×V31−2.83×V32−2.85×V33+0.993×V34+2.321×V35−0.71×V36−0.616×V37−1.711×V38+9.051×V39−1.52×V40+0.302×V41−1.688×V42−0.739×V43−0.152×V44−0.282×V45−0.085×V46+7.81, wherein the cut-off value of P was 0.21, ROC analysis was conducted and as shown in FIG. 10, the AUC was 0.973, and the sensitivity and specificity were 0.920 and 0.941, respectively, which indicated that the model could be used for differential diagnosis of benign pulmonary nodules and malignant tumors. According to the aforementioned screened differential metabolites (Tables 2 to 7), different differential metabolites could be selected to establish a variety of prediction models, all of which might have diagnostic values, and accordingly, the combination of differential metabolites screened out by them also had a diagnostic value.

### EXAMPLE 6

### Application of Model for Differential Diagnosis between Lung Cancer and Benign Pulmonary Nodules

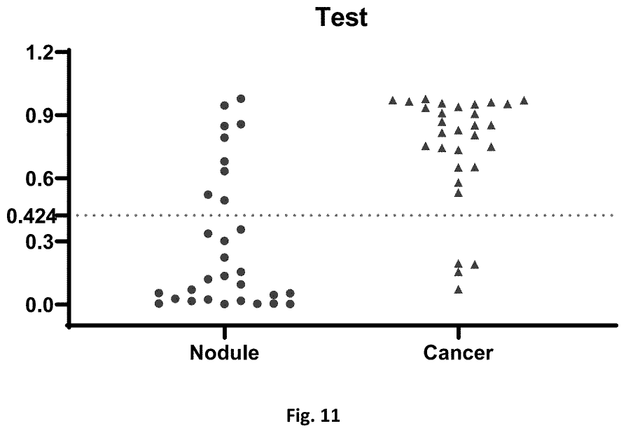

We utilized Model A of Example 5 to predict 30 cases of lung cancer and benign pulmonary nodules that were randomly selected inside and outside the hospital and not participating in establishment of the model. As shown in FIG. 11, the results showed that the prediction accuracy of model A for lung cancer reached 86.7%, and that for benign nodules reached 70%. The results showed that the model for differential diagnosis of lung cancer and benign pulmonary nodules as established by us had higher sensitivity and specificity, and could effectively conduct differential diagnosis of lung cancer and benign pulmonary nodules.

The results here were only preliminary prediction results. If the sample size was increased, the prediction results might be more accurate, but this did not deny that these markers found in the invention were biomarkers that could be used for diagnosing whether suffering from lung cancer.

Screening and Detection of Additional Novel Markers

### EXAMPLE 7

### Collection of Serum Samples

Serum samples were collected from patients of different genders and ages and healthy people. In this study, samples of people aged between 38-78 were collected, including three groups of serum samples from patients with lung cancer (136 cases), patients with benign pulmonary nodules (170 cases) and healthy people (174 cases).

### EXAMPLE 8

### Extraction of Serum Metabolites

Serum metabolites were extracted by a three-phase extraction method of methyl tert-butyl ether:methanol:water (10:3:2.5, v/v/v). The specific operation was as follows: (1) the serum sample was placed on ice and completely thawed, 50 uL of the sample was taken into a 1.5 mL EP tube, added with 225 μL of frozen methanol, and subjected to vortex for 30 seconds; (2) it was added with 750 μL of frozen MTBE, subjected to vortex for 30 seconds, and shaken on ice at 400 rpm for 1 hour; (3) it was then added with 188 μL of pure water and subjected to vortex for 1 minute; (4) it was centrifuged at 15,000 rcf for 10 minutes at 4° C.; and (5) upon centrifugation, 125 μL of the subnatant was taken into an EP tube and spin-dried with a vacuum freeze dryer, and all the dry samples of serum metabolites were stored in a refrigerator at −80° C. until testing.

Considering that there might be batch errors in sample pretreatment, in this study, processing of each batch of experimental samples was conducted simultaneously with the processing of one Reference serum for subsequent data correction. The Reference serum sample was prepared by mixing sera from 100 healthy people (healthy people referred to the people whose blood pressure, blood sugar and blood routine were all normal and that had no hepatitis B virus, and of which the physical examination results showed no obvious diseases, so that they did not need to see a doctor for treatment currently). The men and women from which the sera of 100 healthy people were derived were of the equal number, and were aged between 40-55. The subjects needed to fast overnight and forbid taking drugs 72 hours before blood collection, and individuals with past disease history and body mass indexes (BM's) outside the 95th percentile were excluded. The mixed serum was sub-packaged in 50 μL per portion and stored in a refrigerator at −80° C.

### EXAMPLE 9

### Detection of Extracted Serum Metabolites and Data Preprocessing

(1) Reconstitution of serum metabolites: the dry extract of serum metabolites was added with 120 μL of a reconstitution solvent (acetonitrile: water=4:1), subjected to vortex for 5 minutes, and then centrifuges at 4° C. for 15,000×g for 10 minutes, and 100 μL of the supernatant was taken into a liner tube to prepare a sample to be tested.

(2) QC sample: each 10 μL of the serum samples to be tested from patients with lung cancer, patients with benign pulmonary nodules and healthy people was taken, subjected to vortex, and mixed evenly with shaking to prepare a QC sample.

(3) Sample detection method: detection was conducted with liquid chromatography-high resolution mass spectrometry (LC-HRMS).

I. Liquid Chromatography Conditions

Chromatographic Column: BEH Amide (100×2.1 mm, 1.7 μm).

Mobile phase: in positive mode, phase A was acetonitrile; water=95:5 (10 mM ammonium acetate, 0.1% formic acid), and phase B was acetonitrile: water=50:50 (10 mM ammonium acetate, 0.1% formic acid); and in negative mode, phase A was acetonitrile: water=95:5 (10 mM ammonium acetate, pH =9.0, adjusted by aqueous ammonia), and phase B was acetonitrile: water=50:50 (10 mM ammonium acetate, pH=9.0, adjusted by aqueous ammonia).

The elution gradient was shown in the table below:

TABLE 15
Elution gradient of LC-HRMS mobile phase

| Time (min) | Flow rate (mL/min) | Phase A | Phase B |
|---|---|---|---|
| 0.0 | 0.30 | 98 | 2 |
| 0.50 | 0.30 | 98 | 2 |
| 12.0 | 0.30 | 50 | 50 |
| 14.0 | 0.30 | 2 | 98 |
| 16.0 | 0.30 | 2 | 98 |
| 16.1 | 0.30 | 98 | 2 |
| 20.0 | 0.30 | 98 | 2 |

II. Mass Spectrometry Conditions

The model of a mass spectrometer was Q Exactive (Thermo Fisher Scientific Company, USA), and qualitative analysis was carried out by employing an electrospray ion source (ESI), a positive and negative Fullscan mode (Full Scan) and a data dependent scan mode (ddMS2). The spray voltage was +3,800/−3,200 V; the atomization temperature was 350° C.; high-purity nitrogen was used as sheath gas and auxiliary gas, and the parameters were set to 40 arb and 10 arb; respectively; the temperature of ion transfer tube was 320° C.; the mass scanning range was 70-1,050 m/z; the primary scan resolution was 70,000 FWHM, and the secondary scan resolution was 35,000 FWHM.

III. Injecting Method

Before each detection, six syringe volumes of the QC sample were injected to stabilize the detection system. The serum sample was injected in a random manner, in which testing of one syringe volume of the QC sample was inserted every injection of 10 syringe volumes of the serum samples. The first syringe and last syringe in the detection sequence were both the QC sample. Finally, the QC sample was subjected to full scanning and segmented scanning by ddMS2 for compound identification.

(4) Data Preprocessing

I. Raw Data Matrix

The raw data of each sample included total ion current data and mass spectrum data (as shown in FIG. 2). All the raw data of the sample was imported into Compound Discovery software to get the information of m/z ions and retention time, and the database (mzCloud and Chemspider) was searched to get the identification results of the compound. Further, according to the information of m/z ions and retention time, the chromatographic integration of each sample was carried out by using Tracefinder software, so as to obtain more accurate peak area information. Finally, a two-dimensional data matrix containing characteristic ions (a combination of m/z ions and retention time) and contents thereof (peak area) was obtained for each sample.

II. Excision and Interpolation of Data Missing Values

There were often data missing values in the original data matrix of metabonomics, which were mainly related to the detection of background noise, peak extraction and peak alignment methods of mass spectrometry, etc. Too many zero or missing values would bring difficulties to downstream analysis. Therefore, characteristic ions with missing values greater than 50% in all samples were generally excised, and the missing values of other compounds were interpolated. In this study, MetaboAnalyst 5.0 analysis software was used for processing the missing values, and 1/5 of the minimum value was selected for interpolation.

III. Data Correction and Filtering

A large amount of sample pretreatment was inevitably limited by the throughput of experimental treatment, so it was necessary to carry out sample pretreatment in batches. However, due to the multifarious types of metabolites, large differences in physical and chemical properties, and expensive isotope internal standards, it was difficult to choose an appropriate isotope internal standard that could meet the full coverage. Aiming at this problem, this study selected a reference serum that is processed simultaneously with the batch processing as a natural “like internal standard” to correct batch errors caused by pretreatment. That was, the original data of the experimental samples of each pretreatment batch was normalized based on the data of the Reference serum of the corresponding batch to obtain the relative abundance of each characteristic ion, and the characteristic ion with RSD>30% in the QC sample was deleted to obtain the final analysis data matrix.

### EXAMPLE 10

### Samples were Grouped by Partial Least Squares Discriminant Analysis, and Differential Metabolites of Different Groups Were Screened According to Fold Change and Significance Analysis

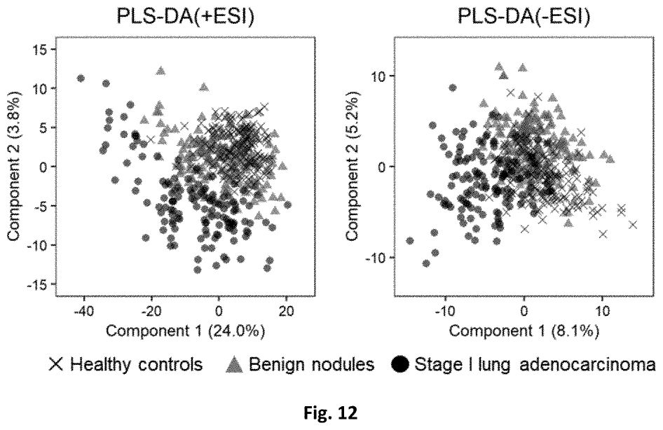

Metabonomics generally adopted the combination of univariate analysis and multivariate statistical analysis to screen differential metabolites, in which the univariate analysis mainly included significance analysis (p value or FDR value) and Fold change of characteristic ions in different groups, while the multivariate statistical analysis mainly included principal component analysis (PCA), partial least squares discriminant analysis (PLS-DA) and orthogonal partial least squares discriminant analysis (OPLS-DA). Before statistical analysis, the data should be properly normalized, transformed and scaled. In this study, MetaboAnalyst 5.0 analysis software was used for statistical analysis, and data normalization by the sum, Log transformation and Auto scaling were carried out. Partial least squares discriminant analysis (PLS-DA) was performed on the three groups of lung cancer, benign pulmonary nodules and healthy people (as shown in FIG. 12), and obvious grouping results were obtained.

According to the screening criteria of the differential metabolite: (1) Fold change>1.2 or <0.83; (2) when FDR<0.05, i.e. Fold change>1.2 or <0.83 and FDR<0.05, it was judged that there was a significant difference in the metabolite between the two groups, and the metabolite was the differential metabolite between the two groups.

It should be noted that when using the method of serum metabolomics to screen differential metabolites, the screened differential metabolites would be affected by many factors, including: the sample size, such as differences in sample sizes and sources of the patients with lung cancer, the patients with benign pulmonary nodules and healthy people in this application, and the like, which each could affect the final results; sample treatment methods, wherein for example different substances would be obtained by using different extraction solvents, which would also lead to different detection results; liquid chromatography and mass spectrometry conditions, wherein the compounds detected under different liquid chromatography and/or mass spectrometry conditions were different; and data analysis methods, wherein differential metabolites obtained by employing different statistical analysis methods would also be different. Furthermore, the effect of the combination of these influencing factors would be more complicated, so it was impossible to predict the results of the final screened differential metabolites. Particularly, in the method of screening lung cancer biomarkers based on serum metabonomics in the present application, the missing value was processed by using MetaboAnalyst 5.0 analysis software, and 1/5 of the minimum value was selected for interpolation. Under the aforementioned screening criteria, even if the number of samples was further increased on the basis of the existing sample size, the obtained differential metabolites hardly changed, which indicated that the screening method in the present application was relatively stable and the obtained differential metabolites had high representativeness. The main differential metabolites found in the present application were shown in the table below.

TABLE 16
Differential metabolites between patients with lung cancer (LA) and healthy people (HC), and
between patients with lung cancer (LA) and patients with benign pulmonary nodules (BN).

| | | | LA vs HC | | LA vs BN | |
|---|---|---|---|---|---|---|
| No. | Name | Class | FC | FDR | FC | FDR |
| 1 | 2-trans,4-cis-Decadienoylcarnitine | Acyl carnitine | 0.59 | 1.07E−13 | 0.74 | 4.15E−05 |
| 2 | Octanoylcarnitine | Acyl carnitine | 0.62 | 3.97E−10 | 0.75 | 4.03E−06 |
| 3 | Decanoylcarnitine | Acyl carnitine | 0.62 | 2.00E−09 | 0.73 | 6.74E−06 |
| 4 | 2-Octenoylcarnitine | Acyl carnitine | 0.69 | 3.22E−05 | 0.69 | 8.57E−06 |
| 5 | Hexanoylcarnitine | Acyl carnitine | 0.69 | 2.95E−07 | 0.82 | 3.89E−04 |
| 6 | 3-hydroxydodecanoyl carnitine | Acyl carnitine | 0.62 | 3.60E−11 | 0.6 | 1.24E−07 |
| 7 | 3-hydroxydecanoyl carnitine | Acyl carnitine | 0.68 | 5.10E−12 | 0.66 | 1.79E−09 |
| 8 | 3-hydroxyoctanoyl carnitine | Acyl carnitine | 0.72 | 5.67E−10 | 0.69 | 3.80E−09 |
| 9 | Ecgonine | Alkaloid | 0.67 | 1.92E−09 | 0.66 | 1.66E−08 |
| 10 | Trimethylamine N-oxide | Amine | 0.52 | 4.92E−09 | 0.69 | 7.48E−06 |
| 11 | 1-Methylnicotinamide | Amine | 0.7 | 3.46E−10 | 0.67 | 1.05E−07 |
| 12 | 3-Chlorotyrosine | Amino acid | 0.46 | 1.09E−08 | 0.6 | 2.89E−06 |
| 13 | Homo-L-arginine | Amino acid | 0.74 | 1.18E−07 | 0.76 | 1.93E−06 |
| 14 | Serotonin | Amino acid | 0.81 | 3.00E−04 | 0.83 | 9.96E−04 |
| 15 | Alanine | Amino acid | 1.23 | 8.28E−06 | 1.24 | 6.18E−06 |
| 16 | alpha-Eleostearic acid | Fatty acid | 0.7 | 2.52E−09 | 0.71 | 1.06E−06 |
| 17 | Ethyl 3-oxohexanoate | Fatty acid | 0.8 | 1.37E−05 | 0.78 | 2.51E−05 |
| 18 | Inosine | Nucleoside | 0.41 | 4.34E−12 | 0.38 | 4.34E−12 |
| 19 | Arabinosylhypoxanthine | Nucleoside | 0.47 | 5.51E−11 | 0.41 | 4.34E−11 |
| 20 | Hippuric acid | Organic acid | 0.52 | 9.60E−09 | 0.64 | 1.93E−06 |
| 21 | Cyclohexaneacetic acid | Organic acid | 0.76 | 2.38E−03 | 0.8 | 7.30E−05 |
| 22 | Ethyl 3-oxohexanoate | Organic acid | 1.24 | 2.23E−07 | 1.3 | 3.59E−10 |
| 23 | 2-Ketobutyric acid | Organic acid | 1.37 | 9.01E−04 | 1.35 | 2.17E−03 |
| 24 | Pyruvic acid | Organic acid | 1.44 | 7.14E−08 | 1.39 | 3.43E−06 |
| 25 | Hypoxanthine | Purine | 1.27 | 1.82E−09 | 1.28 | 1.79E−09 |
| 26 | Xanthine | Purine | 1.28 | 3.08E−07 | 1.28 | 2.77E−06 |
| 27 | N6,N6,N6-Trimethylysine | Amino acid | 0.81 | 2.73E−04 | / | / |
| 28 | Kynurenine | Amino acid | / | / | 0.78 | 4.10E−05 |
| 29 | cis-5-Tetradecenoylcarnitine | Acyl carnitine | / | / | 0.77 | 4.87E−04 |
| 30 | Docosahexaenoic acid | Fatty acid | / | / | 0.78 | 1.50E−05 |
| 31 | Choline Sulfate | Organic acid | / | / | 0.72 | 7.65E−07 |
| 32 | Dihydrothymine | Pyrimidine | / | / | 1.3 | 1.62E−03 |
| 33 | 17-Hydroxypregnenolone sulfate | Sulfated steroid | / | / | 1.22 | 6.73E−03 |
| 34 | Pregnenolone sulfate | Sulfated steroid | / | / | 1.47 | 2.93E−02 |
| 35 | Tiglylcarnitine | Acyl carnitine | 0.82 | 1.56E−04 | / | / |
| 36 | Propionylcarnitine | Acyl carnitine | 0.82 | 3.99E−07 | / | / |
| 37 | 3-hydroxybutyryl carnitine | Acyl carnitine | 0.78 | 2.16E−04 | / | / |
| 38 | Oxindole | Alkaloid | 0.8 | 1.97E−03 | / | / |
| 39 | Nicotine | Alkaloid | 0.83 | 9.43E−03 | / | / |
| 40 | Ergothioneine | Amino acid | 0.79 | 9.12E−04 | / | / |
| 41 | Phenylacetylglutamine | Amino acid | 0.8 | 2.65E−02 | / | / |
| 42 | Citrulline | Amino acid | 0.83 | 3.53E−08 | / | / |
| 43 | Lysine | Amino acid | 0.83 | 6.90E−10 | / | / |
| 44 | Aminocaproic acid | Fatty acid | 1.29 | 4.71E−03 | / | / |
| 45 | Methylimidazoleacetic acid | Organic acid | 0.82 | 1.85E−02 | / | / |

It could be seen from the data in the table that:

(1) The metabolites that had significant differences both between the lung cancer group and the healthy (without nodules) group and between the lung cancer group and the group with benign pulmonary nodules included: 2-trans,4-cis-Decadienoylcarnitine, Octanoylcarnitine, Decanoylcarnitine, 2-Octenoylcarnitine, Hexanoylcarnitine, 3-hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine.

(2) The metabolites that had significant differences only between the lung cancer group and the healthy (without nodules) group, but not between the lung cancer group and the group with benign pulmonary nodules included: N6,N6,N6-Trimethylysine, Tiglylcarnitine, Propionylcarnitine, 3-hydroxybutyrylcarnitine, Oxindole, Nicotine, Ergothioneine, Phenylacetylglutamine, Citrulline, Lysine, Aminocaproic acid, Methylimidazoleacetic acid.

(3) The metabolites that had significant differences only between the lung cancer group and the group with benign pulmonary nodules, but not between the lung cancer group and the healthy (without nodules) group included: Kynurenine, cis-5-Tetradecenoylcarnitine, Docosahexaenoic acid, Choline Sulfate, Dihydrothymine, 17-Hydroxypregnenolone sulfate, Pregnenolone sulfate.

(4) The differential metabolites in the table were classified into types of acyl carnitine, amines, alkaloids, fatty acids, amino acids, etc., which indicated that these types of serum metabolites were highly likely to be related to lung cancer. Moreover, the related metabolic pathways of the differential metabolites in the table and other metabolites in the pathways were also highly likely to be related to lung cancer, so they could be given priority in consideration during the process of finding biomarkers of lung cancer.

### EXAMPLE 11

### Model for Differential Diagnosis of Lung Cancer and Benign Pulmonary Nodules and Establishment thereof

1. The model for differential diagnosis between lung cancer and benign pulmonary nodules or between patients with lung cancer and healthy people by a single differential metabolite and establishment thereof

An ROC curve of each differential metabolite was established, and the quality of the experimental results was judged by the area under the curve (AUC). The AUC less than or equal to 0.5 indicated that the single differential metabolite had no diagnostic value; the AUC greater than 0.5 indicated that the single differential metabolite had a diagnostic value; and the diagnostic value of the single differential metabolite was higher when the AUC was larger.

The respective differential metabolites in Table 16 were analyzed by ROC curve, and the ROC values and related information of them were shown in Tables 17 and 18, respectively:

TABLE 17
ROC values and related information of differential metabolites between lung cancer
samples and healthy (without nodules) samples as obtained by ROC Analysis

| | | | 95% confidence | | | Cut-off |
|---|---|---|---|---|---|---|
| No. | Metabolites | AUC | interval | Sensitivity | Specificity | value |
| 1 | 2-trans,4-cis-Decadienoylcarnitine | 0.746 | 0.698-0.797 | 0.5 | 0.8 | 1.160 |
| 2 | Octanoylcarnitine | 0.692 | 0.631-0.756 | 0.7 | 0.6 | 0.745 |
| 3 | Decanoylcarnitine | 0.688 | 0.622-0.750 | 0.6 | 0.7 | 0.880 |
| 4 | 2-Octenoylcarnitine | 0.627 | 0.560-0.683 | 0.5 | 0.7 | 1.100 |
| 5 | Hexanoylcarnitine | 0.660 | 0.596-0.718 | 0.7 | 0.5 | 0.853 |
| 6 | 3-hydroxydodecanoyl carnitine | 0.713 | 0.651-0.771 | 0.8 | 0.6 | 0.749 |
| 7 | 3-hydroxydecanoyl carnitine | 0.718 | 0.657-0.771 | 0.6 | 0.7 | 0.975 |
| 8 | 3-hydroxyoctanoyl carnitine | 0.692 | 0.634-0.748 | 0.5 | 0.8 | 1.050 |
| 9 | Ecgonine | 0.687 | 0.629-0.744 | 0.8 | 0.5 | 0.664 |
| 10 | Trimethylamine N-oxide | 0.682 | 0.624-0.742 | 0.9 | 0.4 | 0.327 |
| 11 | 1-Methylnicotinamide | 0.681 | 0.617-0.735 | 0.9 | 0.4 | 0.688 |
| 12 | 3-Chlorotyrosine | 0.708 | 0.649-0.757 | 0.8 | 0.5 | 0.127 |
| 13 | Homo-L-arginine | 0.645 | 0.585-0.712 | 0.6 | 0.7 | 0.920 |
| 14 | Serotonin | 0.581 | 0.521-0.643 | 0.5 | 0.6 | 0.977 |
| 15 | Alanine | 0.591 | 0.527-0.657 | 0.5 | 0.7 | 1.170 |
| 16 | alpha-Eleostearic acid | 0.687 | 0.620-0.743 | 0.6 | 0.8 | 1.010 |
| 17 | Ethyl 3-oxohexanoate | 0.686 | 0.626-0.743 | 0.8 | 0.5 | 0.683 |
| 18 | Inosine | 0.733 | 0.677-0.786 | 0.7 | 0.7 | 0.405 |
| 19 | Arabinosylhypoxanthine | 0.748 | 0.697-0.800 | 0.8 | 0.7 | 0.408 |
| 20 | Hippuric acid | 0.711 | 0.650-0.770 | 0.8 | 0.6 | 0.205 |
| 21 | Cyclohexaneacetic acid | 0.629 | 0.568-0.691 | 0.6 | 0.6 | 0.876 |
| 22 | Ethyl 3-oxohexanoate | 0.609 | 0.541-0.674 | 0.5 | 0.7 | 0.993 |
| 23 | 2-Ketobutyric acid | 0.585 | 0.525-0.640 | 0.3 | 0.9 | 0.747 |
| 24 | Pyruvic acid | 0.640 | 0.576-0.702 | 0.4 | 0.8 | 0.968 |
| 25 | Hypoxanthine | 0.630 | 0.578-0.687 | 0.6 | 0.6 | 1.010 |
| 26 | Xanthine | 0.604 | 0.538-0.659 | 0.8 | 0.4 | 1.230 |
| 27 | N6,N6,N6-Trimethylysine | 0.585 | 0.525-0.649 | 0.7 | 0.5 | 0.889 |
| 28 | Tiglylcarnitine | 0.594 | 0.531-0.652 | 0.8 | 0.4 | 0.808 |
| 29 | Propionylcarnitine | 0.612 | 0.558-0.675 | 0.9 | 0.3 | 0.789 |
| 30 | 3-hydroxybutyryl carnitine | 0.606 | 0.533-0.673 | 0.6 | 0.6 | 0.872 |
| 31 | Oxindole | 0.578 | 0.518-0.640 | 0.5 | 0.7 | 1.030 |
| 32 | Nicotine | 0.545 | 0.483-0.608 | 0.6 | 0.5 | 0.792 |
| 33 | Ergothioneine | 0.598 | 0.534-0.665 | 0.9 | 0.3 | 0.448 |
| 34 | Phenylacetylglutamine | 0.556 | 0.494-0.620 | 0.9 | 0.2 | 0.279 |
| 35 | Citrulline | 0.616 | 0.550-0.686 | 0.7 | 0.6 | 0.968 |
| 36 | Lysine | 0.646 | 0.582-0.702 | 0.6 | 0.7 | 1.020 |
| 37 | Aminocaproic acid | 0.612 | 0.548-0.671 | 0.5 | 0.8 | 0.369 |
| 38 | Methylimidazoleacetic acid | 0.556 | 0.493-0.623 | 0.6 | 0.5 | 0.723 |

TABLE 18
ROC values and related information of differential metabolites between lung cancer
samples and samples with benign pulmonary nodules as obtained by ROC Analysis

| | | | 95% confidence | | | Cut-off |
|---|---|---|---|---|---|---|
| No. | Metabolites | AUC | interval | Sensitivity | Specificity | value |
| 1 | 2-trans,4-cis-Decadienoylcarnitine | 0.652 | 0.592-0.708 | 0.7 | 0.5 | 0.761 |
| 2 | Octanoylcarnitine | 0.670 | 0.605-0.722 | 0.7 | 0.6 | 0.761 |
| 3 | Decanoylcarnitine | 0.657 | 0.591-0.718 | 0.7 | 0.6 | 0.692 |
| 4 | 2-Octenoylcarnitine | 0.648 | 0.586-0.700 | 0.6 | 0.7 | 1.070 |
| 5 | Hexanoylcarnitine | 0.623 | 0.554-0.683 | 0.9 | 0.3 | 0.661 |
| 6 | 3-hydroxydodecanoyl carnitine | 0.682 | 0.621-0.735 | 0.8 | 0.5 | 0.674 |
| 7 | 3-hydroxydecanoyl carnitine | 0.711 | 0.641-0.767 | 0.7 | 0.6 | 0.834 |
| 8 | 3-hydroxyoctanoyl carnitine | 0.700 | 0.641-0.761 | 0.7 | 0.6 | 0.826 |
| 9 | Ecgonine | 0.701 | 0.642-0.751 | 0.6 | 0.8 | 0.905 |
| 10 | Trimethylamine N-oxide | 0.651 | 0.586-0.710 | 0.8 | 0.4 | 0.344 |
| 11 | 1-Methylnicotinamide | 0.674 | 0.607-0.731 | 0.7 | 0.5 | 0.828 |
| 12 | 3-Chlorotyrosine | 0.683 | 0.623-0.747 | 0.8 | 0.6 | 0.145 |
| 13 | Homo-L-arginine | 0.654 | 0.597-0.708 | 0.6 | 0.7 | 0.919 |
| 14 | Serotonin | 0.597 | 0.529-0.658 | 0.9 | 0.2 | 0.526 |
| 15 | Alanine | 0.567 | 0.497-0.628 | 0.5 | 0.6 | 1.240 |
| 16 | alpha-Eleostearic acid | 0.674 | 0.614-0.731 | 0.6 | 0.7 | 0.965 |
| 17 | Ethyl 3-oxohexanoate | 0.687 | 0.629-0.746 | 0.5 | 0.7 | 0.935 |
| 18 | Inosine | 0.746 | 0.693-0.794 | 0.7 | 0.7 | 0.400 |
| 19 | Arabinosylhypoxanthine | 0.766 | 0.710-0.819 | 0.7 | 0.7 | 0.505 |
| 20 | Hippuric acid | 0.692 | 0.629-0.754 | 0.8 | 0.5 | 0.206 |
| 21 | Cyclohexaneacetic acid | 0.671 | 0.603-0.723 | 0.5 | 0.8 | 1.160 |
| 22 | Ethyl 3-oxohexanoate | 0.627 | 0.560-0.638 | 0.8 | 0.4 | 1.270 |
| 23 | 2-Ketobutyric acid | 0.561 | 0.493-0.626 | 0.9 | 0.2 | 2.140 |
| 24 | Pyruvic acid | 0.600 | 0.537-0.663 | 0.6 | 0.6 | 1.220 |
| 25 | Hypoxanthine | 0.611 | 0.541-0.670 | 0.6 | 0.6 | 0.970 |
| 26 | Xanthine | 0.566 | 0.502-0.621 | 0.7 | 0.4 | 1.190 |
| 27 | Kynurenine | 0.624 | 0.558-0.688 | 0.5 | 0.7 | 1.330 |
| 28 | cis-5-Tetradecenoylcarnitine | 0.617 | 0.546-0.676 | 0.8 | 0.4 | 0.752 |
| 29 | Docosahexaenoic acid | 0.693 | 0.635-0.754 | 0.7 | 0.6 | 0.932 |
| 30 | Choline Sulfate | 0.666 | 0.608-0.730 | 0.6 | 0.7 | 0.762 |
| 31 | Dihydrothymine | 0.565 | 0.496-0.630 | 0.7 | 0.4 | 1.140 |
| 32 | 17-Hydroxypregnenolone sulfate | 0.533 | 0.468-0.599 | 0.7 | 0.4 | 0.999 |
| 33 | Pregnenolone sulfate | 0.530 | 0.470-0.592 | 0.3 | 0.8 | 0.536 |

The ROC values of the differential metabolites between lung cancer samples and healthy (without nodules) samples in Table 3 were all equal to and greater than 0.5, indicating that these differential metabolites had a certain value for distinguishing the lung cancer samples from the healthy (without nodules) samples, and could be used as biomarkers for the auxiliary diagnosis of lung cancer. The higher AUC value in the table indicated the higher diagnostic value of the single biomarker, and the greater value or reference significance of it that might be obtained when it was used for diagnosis alone or in combination with multiple biomarkers. Meanwhile, for biomarkers with relatively small AUC values, their diagnostic or identifying significance cannot be denied, and use of single ones of them also provided a possibility or reference to a certain extent. Moreover, when multiple biomarkers with relatively small AUC values were combined together, the value of combined diagnosis was often higher, even reaching the accuracy of 80%, 90% and above. Therefore, any biomarker in Table 17 had more or less significance in distinguishing the lung cancer samples from the health (without nodules) samples or auxiliary diagnosis of lung cancer. Similarly, the biomarkers in Table 18 had the same value or significance for the diagnosis or identification of benign pulmonary nodules and lung cancer.

2. The model for identifying lung cancer samples and healthy (without nodules) samples or samples with benign pulmonary nodules by a combination of multiple differential metabolites and establishment thereof

Based on the serum detection values of differential metabolites in the lung cancer samples and the samples with pulmonary nodules in Table 15, a model for differential diagnosis between the lung cancer samples and the healthy (without nodules) samples or between the lung cancer samples and the samples with benign pulmonary nodules was established by utilizing binary logistic regression (LASSO algorithm, R language). The models for distinguishing lung cancer from benign pulmonary nodules included model A, model B, model C and model D. The models for distinguishing individuals with lung cancer from healthy individuals included: model E, model F, model G, model H and model I.

I. The variables and parameters of model A were listed in the table below:

TABLE 19
List of variables and parameters of model A

| No. | Metabolites | Weights | Odds ratio |
|---|---|---|---|
| M1 | Hypoxanthine | 2.29 | 9.87 |
| M2 | Alanine | 1.02 | 2.77 |
| M3 | 2-Ketobutyric acid | 0.64 | 1.90 |
| M4 | 2-trans,4-cis-Decadienoylcarnitine | 0.62 | 1.86 |
| M5 | Xanthine | 0.47 | 1.60 |
| M6 | 17-Hydroxypregnenolone sulfate | 0.42 | 1.52 |
| M7 | Dihydrothymine | 0.38 | 1.46 |
| M8 | Octanoylcarnitine | 0.26 | 1.30 |
| M9 | Ethyl 3-oxohexanoate | 0.05 | 1.05 |
| M10 | Pregnenolone sulfate | 0.03 | 1.03 |
| M11 | 3-Chlorotyrosine | −0.05 | 0.95 |
| M12 | Cyclohexaneacetic acid | −0.12 | 0.89 |
| M13 | Choline Sulfate | −0.16 | 0.85 |
| M14 | Trimethylamine N-oxide | −0.17 | 0.84 |
| M15 | 2-Octenoylcarnitine | −0.36 | 0.70 |
| M16 | 1-Methylnicotinamide | −0.40 | 0.67 |
| M17 | Serotonin | −0.45 | 0.64 |
| M18 | Docosahexaenoic acid | −0.46 | 0.63 |
| M19 | Decanoylcarnitine | −0.47 | 0.63 |
| M20 | alpha-Eleostearic acid | −0.53 | 0.59 |
| M21 | Homo-L-arginine | −0.55 | 0.58 |
| M22 | Pyruvic acid | −0.79 | 0.45 |
| M23 | 3-hydroxydecanoyl carnitine | −0.95 | 0.39 |
| M24 | Ecgonine | −1.02 | 0.36 |
| M25 | Kynurenine | −1.19 | 0.30 |
| M26 | Ethyl 3-oxohexanoate | −1.52 | 0.22 |
| M27 | Arabinosylhypoxanthine | −1.88 | 0.15 |
| | constant | 4.01 | / |

The equation of model A was: In[P/(1−P)]=2.29×M1+1.02×M2+0.64×M3+0.62×M4+0.47×M5+0.42×M6+0.38×M7+0.26×M8+0.05×M9+0.03×M10−0.05×M11−0.12×M12−0.16×M13−0.17×M14−0.36×M15−0.4×M16−0.45×M17−0.46×M18−0.47×M19−0.53×M20−0.55×M21−0.79×M22−0.95×M23−1.02×M24−1.19×M25−1.52 ×M26−1.88×M27+4.01.

The cut-off value of P was 0.455. That was, the serum detection values (relative abundances) of the aforementioned markers was substituted into the equation of model A for calculation. When P>0.455, it was identified as lung cancer; and when P≤0.455, it was identified as benign pulmonary nodules.

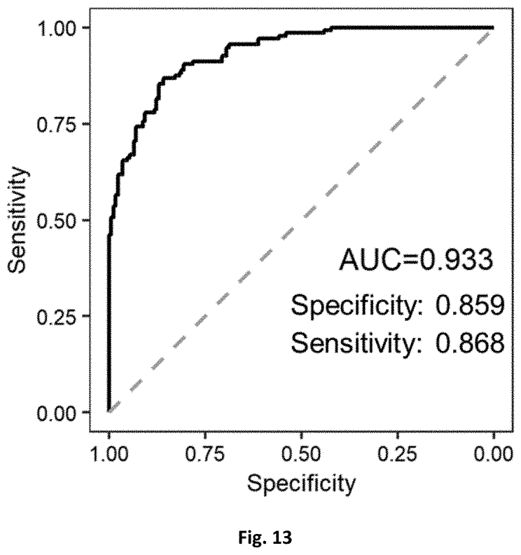

As shown in FIG. 13, ROC analysis was conducted, and the model A had a AUC of 0.933, and specificity and sensitivity of 0.859 and 0.868 respectively.

II. The variables and parameters of model B were listed in the table below:

TABLE 20
List of variables and parameters of model B

| No. | Metabolites | Weights | Odds ratio |
|---|---|---|---|
| M1 | Hypoxanthine | 1.11 | 3.03 |
| M2 | Alanine | 0.25 | 1.28 |
| M3 | 2-Ketobutyric acid | 0.13 | 1.14 |
| M4 | 17-Hydroxypregnenolone sulfate | 0.09 | 1.09 |
| M5 | Dihydrothymine | 0.05 | 1.05 |
| M6 | Trimethylamine N-oxide | −0.01 | 0.99 |
| M7 | Serotonin | −0.02 | 0.98 |
| M8 | 3-Chlorotyrosine | −0.02 | 0.98 |
| M9 | Hippuric acid | −0.04 | 0.96 |
| M10 | Docosahexaenoic acid | −0.11 | 0.90 |
| M11 | 2-Octenoylcarnitine | −0.12 | 0.89 |
| M12 | 1-Methylnicotinamide | −0.19 | 0.83 |
| M13 | Homo-L-arginine | −0.30 | 0.74 |
| M14 | alpha-Eleostearic acid | −0.34 | 0.71 |
| M15 | Kynurenine | −0.45 | 0.64 |
| M16 | 3-hydroxydecanoyl carnitine | −0.46 | 0.63 |
| M17 | Ecgonine | −0.64 | 0.53 |
| M18 | Ethyl 3-oxohexanoate | −0.68 | 0.51 |
| M19 | Arabinosylhypoxanthine | −0.95 | 0.39 |
| | constant | 2.17 | / |

The equation of model B was: In[P/(1−P)]=1.11×M1+0.25×M2+0.13×M3+0.09×M4 +0.05×M5−0.01×M6−0.02×M7−0.02×M8−0.04×M9−0.11×M10−0.12×M11−0.19×M12−0.3×M13−0.34×M14−0.45×M15−0.46×M16−0.64×M17−0.68×M18−0.95×M19+2.17.

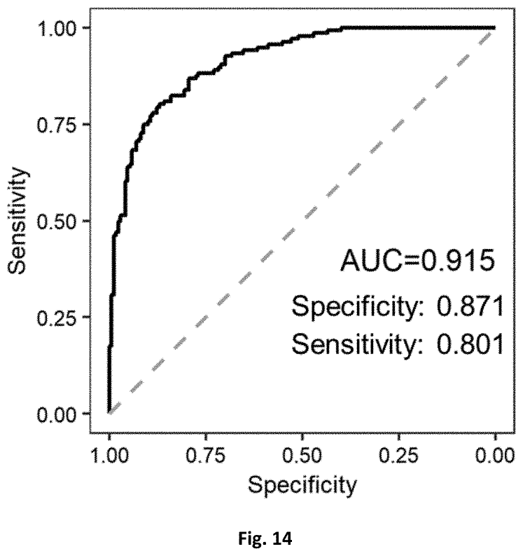

The cut-off value of P was 0.511. That was, the serum detection values (relative abundances) of the aforementioned markers was substituted into the equation of model B for calculation. When P>0.511, it was identified as lung cancer; and when P≤0.511, it was identified as benign pulmonary nodules. ROC analysis was conducted (as shown in FIG. 14), and the model C had a AUC of 0.915, and specificity and sensitivity of 0.871 and 0.801 respectively.

III. The variables and parameters of model C were listed in the table below:

TABLE 21
List of variables and parameters of model C

| No. | Metabolites | Weights | Odds ratio |
|---|---|---|---|
| M1 | Hypoxanthine | 1.73 | 5.64 |
| M2 | Alanine | 0.72 | 2.05 |
| M3 | 2-Ketobutyric acid | 0.31 | 1.36 |
| M4 | 17-Hydroxypregnenolone sulfate | 0.29 | 1.34 |
| M5 | Dihydrothymine | 0.23 | 1.26 |
| M6 | Xanthine | 0.15 | 1.16 |
| M7 | 3-Chlorotyrosine | −0.05 | 0.95 |
| M8 | Cyclohexaneacetic acid | −0.07 | 0.93 |
| M9 | Choline Sulfate | −0.07 | 0.93 |
| M10 | Decanoylcarnitine | −0.09 | 0.91 |
| M11 | Trimethylamine N-oxide | −0.09 | 0.91 |
| M12 | 2-Octenoylcarnitine | −0.16 | 0.85 |
| M13 | PyruMic acid | −0.26 | 0.77 |
| M14 | Docosahexaenoic acid | −0.26 | 0.77 |
| M15 | 1-Methylnicotinamide | −0.27 | 0.76 |
| M16 | Serotonin | −0.29 | 0.75 |
| M17 | Homo-L-arginine | −0.43 | 0.65 |
| M18 | alpha-Eleostearic acid | −0.45 | 0.64 |
| M19 | 3-hydroxydecanoyl carnitine | −0.56 | 0.57 |
| M20 | Ecgonine | −0.75 | 0.47 |
| M21 | Kynurenine | −0.87 | 0.42 |
| M22 | Ethyl 3-oxohexanoate | −1.15 | 0.32 |
| M23 | Arabinosylhypoxanthine | −1.41 | 0.24 |
| | constant | 3.07 | / |

The equation of model C was: In[P/(1−P)]=1.73×M1+0.72×M2+0.31×M3+0.29×M4+0.23×M5+0.15×M6−0.05×M7−0.07×M8−0.07×M9−0.09×M10−0.09×M11−0.16×M12−0.26×M13−0.26×M14−0.27×M15−0.29×M16−0.43×M17−0.45×M18−0.56×M19−0.75×M20−0.87×M21−1.15×M22−1.41×M23+3.07.

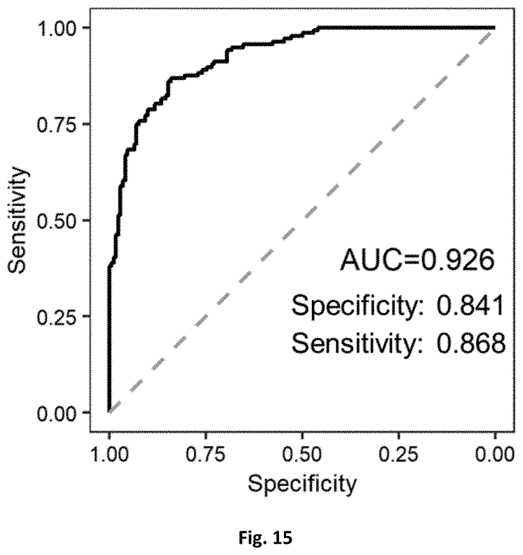

The cut-off value of P was 0.452. That was, the serum detection values (relative abundances) of the aforementioned markers was substituted into the equation of model C for calculation. When P>0.452, it was identified as lung cancer; and when P≤0.452, it was identified as benign pulmonary nodules. ROC analysis was conducted (as shown in FIG. 15), and the model C had a AUC of 0.926, and specificity and sensitivity of 0.841 and 0.868 respectively.

IV. The variables and parameters of model D were listed in the table below:

TABLE 22
List of variables and parameters of model D

| No. | Metabolites | Weights | Odds ratio |
|---|---|---|---|
| M1 | Hypoxanthine | 2.35 | 10.49 |
| M2 | Alanine | 1.03 | 2.80 |
| M3 | 2-Ketobutyric acid | 0.71 | 2.03 |
| M4 | 2-trans,4-cis-Decadienoylcarnitine | 0.68 | 1.97 |
| M5 | Xanthine | 0.48 | 1.62 |
| M6 | Octanoylcarnitine | 0.45 | 1.57 |
| M7 | 17-Hydroxypregnenolone sulfate | 0.42 | 1.52 |
| M8 | Dihydrothymine | 0.39 | 1.48 |
| M9 | Lactic acid | 0.16 | 1.17 |
| M10 | Pregnenolone sulfate | 0.03 | 1.03 |
| M11 | 3-Chlorotyrosine | −0.05 | 0.95 |
| M12 | Cyclohexaneacetic acid | −0.12 | 0.89 |
| M13 | Choline Sulfate | −0.17 | 0.84 |
| M14 | Trimethylamine N-oxide | −0.17 | 0.84 |
| M15 | Hexanoylcarnitine | −0.22 | 0.80 |
| M16 | 2-Octenoylcarnitine | −0.39 | 0.68 |
| M17 | 1-Methylnicotinamide | −0.43 | 0.65 |
| M18 | Serotonin | −0.46 | 0.63 |
| M19 | Docosahexaenoic acid | −0.49 | 0.61 |
| M20 | Decanoylcarnitine | −0.54 | 0.58 |
| M21 | alpha-Eleostearic acid | −0.54 | 0.58 |
| M22 | Homo-L-arginine | −0.57 | 0.57 |
| M23 | Pyruvic acid | −0.89 | 0.41 |
| M24 | 3-hydroxydecanoyl carnitine | −0.97 | 0.38 |
| M25 | Ecgonine | −1.07 | 0.34 |
| M26 | Kynurenine | −1.23 | 0.29 |
| M27 | Ethyl 3-oxohexanoate | −1.56 | 0.21 |
| M28 | Arabinosylhypoxanthine | −1.93 | 0.15 |
| | constant | 4.16 | / |

The equation of model D was: In[P/(1−P)]=2.35×M1+1.03×M2+0.71×M3+0.68×M4+0.48×M5+0.45×M6+0.42×M7+0.39×M8+0.16×M9+0.03×M10−0.05×M11−0.12×M12−0.17×M13−0.17×M14−0.22×M15−0.39×M16−0.43×M17−0.46×M18−0.49×M19−0.54×M20−0.54×M21−0.57×M22−0.89×M23−0.97×M24−1.07×M25−1.23×M26−1.56×M27−1.93×M28+4.16.

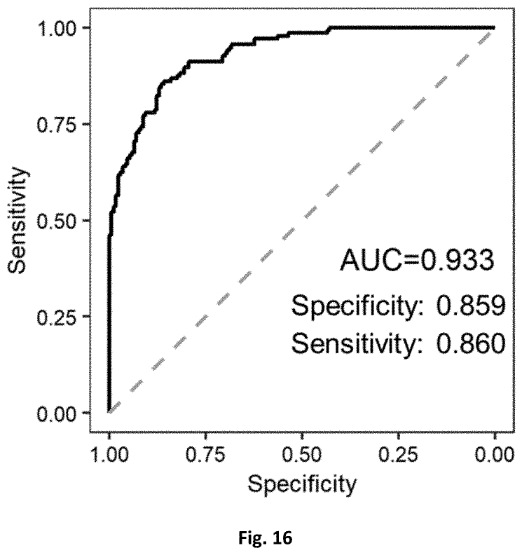

The cut-off value of P was 0.458. That was, the serum detection values (relative abundances) of the aforementioned markers was substituted into the equation of model D for calculation. When P>0.458, it was identified as lung cancer; and when P≤0.458, it was identified as benign pulmonary nodules. ROC analysis was conducted (as shown in FIG. 16), and the model D had a AUC of 0.933, and specificity and sensitivity of 0.859 and 0.860 respectively.

V. The variables and parameters of model E were listed in the table below:

TABLE 23
List of variables and parameters of model E

| No. | Metabolites | Weights | Odds ratio |
|---|---|---|---|
| V1 | Hypoxanthine | 1.41 | 4.10 |
| V2 | Alanine | 0.26 | 1.30 |
| V3 | 2-Ketobutyric acid | 0.04 | 1.04 |
| V4 | 3-hydroxybutyryl carnitine | −0.01 | 0.99 |
| V5 | Nicotine | −0.05 | 0.95 |
| V6 | Hippuric acid | −0.09 | 0.91 |
| V7 | Citrulline | −0.19 | 0.83 |
| V8 | Trimethylamine N-oxide | −0.20 | 0.82 |
| V9 | alpha-Eleostearic acid | −0.32 | 0.73 |
| V10 | 1-Methylnicotinamide | −0.34 | 0.71 |
| V11 | 3-hydroxydecanoyl carnitine | −0.40 | 0.67 |
| V12 | Ecgonine | −0.48 | 0.62 |
| V13 | Ethyl 3-oxohexanoate | −0.55 | 0.58 |
| V14 | 2-trans,4-cis-Decadienoylcarnitine | −0.64 | 0.53 |
| V15 | Arabinosylhypoxanthine | −1.07 | 0.34 |
| V16 | Lysine | −1.58 | 0.21 |
| | constant | 3.44 | / |

The equation of model E was: In[P/(1−P)]=In[P/(1−P)]=1.41×V1+0.26×V2+0.04×V3−0.01×V4−0.05×V5−0.09×V6−0.19×V7−0.2×V8−0.32×V9−0.34×V10−0.4×V11−0.48×V12−0.55×V13−0.64×V14−1.07×V15−1.58×V16+3.44.

The cut-off value of P was 0.520. That was, the serum detection values (relative abundances) of the aforementioned markers was substituted into the equation of model E for calculation. When P>0.520, it was identified as lung cancer; and when P≤0.520, it was identified as healthy people.

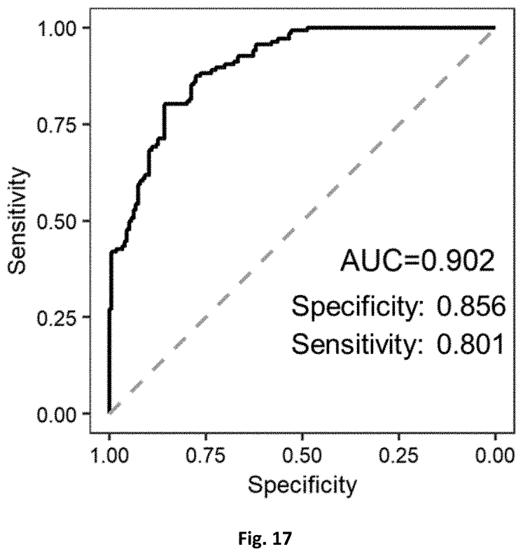

As shown in FIG. 17, ROC analysis was conducted on the samples of model E, the AUC was 0.902, and the sensitivity and specificity were 0.801 and 0.856, respectively, indicating that the model E had high accuracy in distinguishing individuals with lung malignant tumors from healthy individuals.

VI. The variables and parameters of model F were listed in the table below:

TABLE 24
List of variables and parameters of model F

| No. | Metabolites | Weights | Odds ratio |
|---|---|---|---|
| V1 | Hypoxanthine | 1.51 | 4.53 |
| V2 | Alanine | 0.29 | 1.34 |
| V3 | 2-Ketobutyric acid | 0.06 | 1.06 |
| V4 | 3-hydroxybutyryl carnitine | −0.03 | 0.97 |
| V5 | Decanoylcarnitine | −0.03 | 0.97 |
| V6 | Ergothioneine | −0.03 | 0.97 |
| V7 | Nicotine | −0.07 | 0.93 |
| V8 | Hippuric acid | −0.10 | 0.90 |
| V9 | Trimethylamine N-oxide | −0.21 | 0.81 |
| V10 | Citrulline | −0.22 | 0.80 |
| V11 | alpha-Eleostearic acid | −0.33 | 0.72 |
| V12 | 1-Methylnicotinamide | −0.35 | 0.70 |
| V13 | 3-hydroxydecanoyl carnitine | −0.39 | 0.68 |
| V14 | Ecgonine | −0.51 | 0.60 |
| V15 | Ethyl 3-oxohexanoate | −0.59 | 0.55 |
| V16 | 2-trans,4-cis-Decadienoylcarnitine | −0.63 | 0.53 |
| V17 | Arabinosylhypoxanthine | −1.12 | 0.33 |
| V18 | Lysine | −1.69 | 0.18 |
| | constant | 3.65 | / |

The equation of model F was: In[P/(1−P)]=1.51×V1+0.29×V2+0.06×V3−0.03×V4−0.03×V5−0.03×V6−0.07×V7−0.1×V8−0.21×V9−0.22×V10−0.33×V11−0.35×V12−0.39×V13−0.51×V14−0.59×V15−0.63×V16−1.12×V17−1.69×V18+3.65.

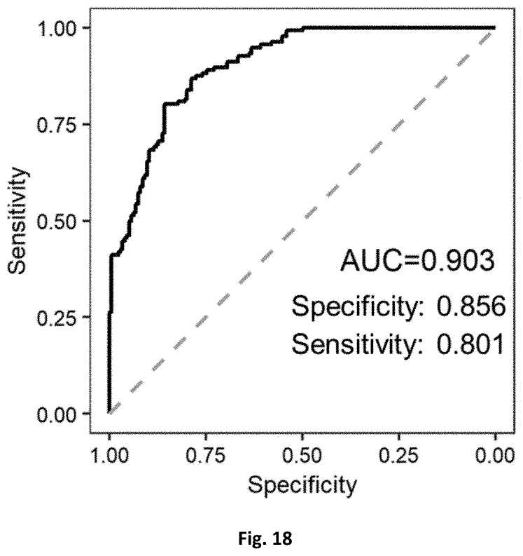

The cut-off value of P was 0.527. That was, the serum detection values (relative abundances) of the aforementioned markers was substituted into the equation of model F for calculation. When P>0.527, it was identified as lung cancer; and when P≤0.527, it was identified as healthy people. ROC analysis was conducted (as shown in FIG. 18), and the model F had a AUC of 0.903, and specificity and sensitivity of 0.856 and 0.801 respectively.

VII. The variables and parameters of model G were listed in the table below:

TABLE 25
List of variables and parameters of model G

| No. | Metabolites | Weights | Odds ratio |
|---|---|---|---|
| V1 | Hypoxanthine | 1.67 | 5.31 |
| V2 | Alanine | 0.34 | 1.40 |
| V3 | 2-Ketobutyric acid | 0.10 | 1.11 |
| V4 | Aminocaproic acid | 0.01 | 1.01 |
| V5 | 3-hydroxybutyryl carnitine | −0.08 | 0.92 |
| V6 | Ergothioneine | −0.08 | 0.92 |
| V7 | Decanoylcarnitine | −0.09 | 0.91 |
| V8 | Nicotine | −0.10 | 0.90 |
| V9 | Hippuric acid | −0.12 | 0.89 |
| V10 | Trimethylamine N-oxide | −0.23 | 0.79 |
| V11 | Citrulline | −0.27 | 0.76 |
| V12 | 3-hydroxydecanoyl carnitine | −0.36 | 0.70 |
| V13 | alpha-Eleostearic acid | −0.36 | 0.70 |
| V14 | 1-Methylnicotinamide | −0.38 | 0.68 |
| V15 | Ecgonine | −0.56 | 0.57 |
| V16 | 2-trans,4-cis-Decadienoylcarnitine | −0.61 | 0.54 |
| V17 | Ethyl 3-oxohexanoate | −0.66 | 0.52 |
| V18 | Arabinosylhypoxanthine | −1.20 | 0.30 |
| V19 | Lysine | −1.89 | 0.15 |
| | constant | 4.00 | / |

The equation of model G was: In[P/(1−P)]=1.67×V1+0.34×V2+0.1×V3+0.01×V4−0.08×V5−0.08×V6−0.09×V7−0.1×V8−0.12×V9−0.23×V10−0.27×V11−0.36×V12−0.36×V13−0.38×V14−0.56×V15−0.61×V16−0.66×V17−1.2×V18−1.89×V19+4.

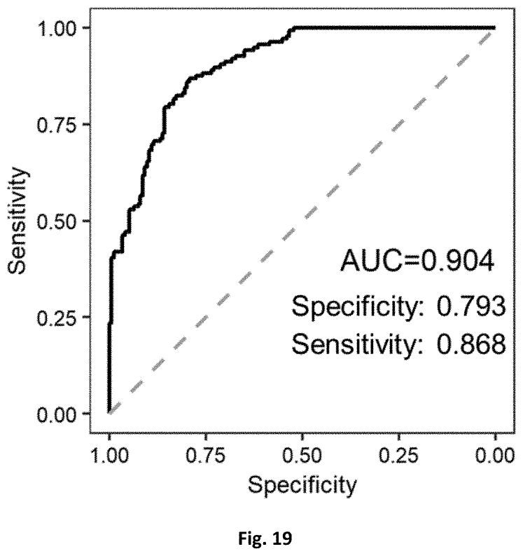

The cut-off value of P was 0.450. That was, the serum detection values (relative abundances) of the aforementioned markers was substituted into the equation of model G for calculation. When P>0.450, it was identified as lung cancer; and when P≤0.450, it was identified as healthy people. ROC analysis was conducted (as shown in FIG. 19), and the model G had a AUC of 0.904, and specificity and sensitivity of 0.793 and 0.868 respectively.

IX. The variables and parameters of model H were listed in the table below:

TABLE 26
List of variables and parameters of model H

| No. | Metabolites | Weights | Odds ratio |
|---|---|---|---|
| V1 | Hypoxanthine | 2.03 | 7.61 |
| V2 | Alanine | 0.47 | 1.60 |
| V3 | 2-Ketobutyric acid | 0.15 | 1.16 |
| V4 | Tiglylcarnitine | 0.09 | 1.09 |
| V5 | N6,N6,N6-Trimethylysine | 0.04 | 1.04 |
| V6 | Aminocaproic acid | 0.03 | 1.03 |
| V7 | Oxindole | 0.01 | 1.01 |
| V8 | Decanoylcarnitine | −0.12 | 0.89 |
| V9 | Nicotine | −0.12 | 0.89 |
| V10 | Ergothioneine | −0.13 | 0.88 |
| V11 | 3-hydroxybutyryl carnitine | −0.14 | 0.87 |
| V12 | Hippuric acid | −0.14 | 0.87 |
| V13 | Trimethylamine N-oxide | −0.27 | 0.76 |
| V14 | Lactic acid | −0.36 | 0.70 |
| V15 | Citrulline | −0.37 | 0.69 |
| V16 | alpha-Eleostearic acid | −0.37 | 0.69 |
| V17 | 3-hydroxydecanoyl carnitine | −0.40 | 0.67 |
| V18 | 1-Methylnicotinamide | −0.43 | 0.65 |
| V19 | 2-trans,4-cis-Decadienoylcarnitine | −0.59 | 0.55 |
| V20 | Ecgonine | −0.63 | 0.53 |
| V21 | Ethyl 3-oxohexanoate | −0.75 | 0.47 |
| V22 | Arabinosylhypoxanthine | −1.37 | 0.25 |
| V23 | Lysine | −2.18 | 0.11 |
| | constant | 4.56 | / |

The equation of model H was: In[P/(1−P)]=2.03×V1+0.47×V2+0.15×V3+0.09×V4+0.04×V5+0.03×V6+0.01×V7−0.12×V8−0.12×V9−0.13×V10−0.14×V11−0.14×V12−0.27×V13−0.36×V14−0.37×V15−0.37×V16−0.4×V17−0.43×V18−0.59×V19−0.63×V20−0.75×V21−1.37×V22−2.18×V23+4.56.

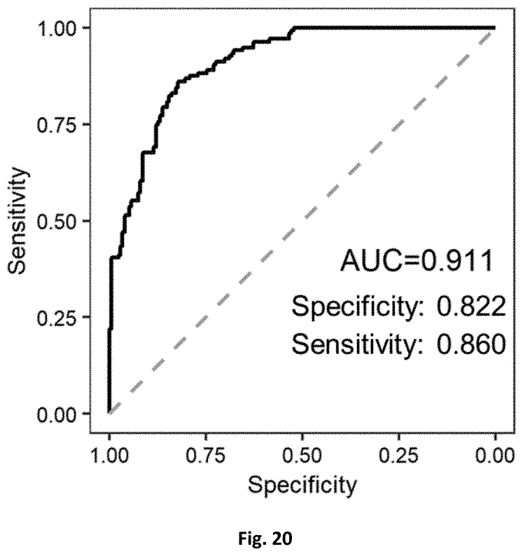

The cut-off value of P was 0.466. That was, the serum detection values (relative abundances) of the aforementioned markers was substituted into the equation of model H for calculation. When P>0.466, it was identified as lung cancer; and when P≤0.466, it was identified as healthy people. ROC analysis was conducted (as shown in FIG. 20), and the model H had a AUC of 0.911, and specificity and sensitivity of 0.822 and 0.860 respectively.

X. The variables and parameters of model I were listed in the table below:

TABLE 27
List of variables and parameters of model I

| No. | Metabolites | Weights | Odds ratio |
|---|---|---|---|
| V1 | Hypoxanthine | 3.08 | 21.76 |
| V2 | Octanoylcarnitine | 1.26 | 3.53 |
| V3 | Alanine | 0.70 | 2.01 |
| V4 | 3-hydroxydodecanoyl carnitine | 0.64 | 1.90 |
| V5 | Xanthine | 0.41 | 1.51 |
| V6 | 2-Ketobutyric acid | 0.40 | 1.49 |
| V7 | Oxindole | 0.38 | 1.46 |
| V8 | Tiglylcarnitine | 0.31 | 1.36 |
| V9 | N6,N6,N6-Trimethylysine | 0.31 | 1.36 |
| V10 | Cyclohexaneacetic acid | 0.10 | 1.11 |
| V11 | Aminocaproic acid | 0.09 | 1.09 |
| V12 | Methylimidazoleacetic acid | 0.09 | 1.09 |
| V13 | Homo-L-arginine | 0.04 | 1.04 |
| V14 | Pyruvic acid | −0.04 | 0.96 |
| V15 | 2-Octenoylcarnitine | −0.04 | 0.96 |
| V16 | Propionylcarnitine | −0.07 | 0.93 |
| V17 | Nicotine | −0.12 | 0.89 |
| V18 | Serotonin | −0.17 | 0.84 |
| V19 | Phenylacetylglutamine | −0.24 | 0.79 |
| V20 | Hippuric acid | −0.24 | 0.79 |
| V21 | Ergothioneine | −0.26 | 0.77 |
| V22 | 3-hydroxybutyryl carnitine | −0.31 | 0.73 |
| V23 | alpha-Eleostearic acid | −0.32 | 0.73 |
| V24 | Inosine | −0.44 | 0.64 |
| V25 | Citrulline | −0.44 | 0.64 |
| V26 | Trimethylamine N-oxide | −0.49 | 0.61 |
| V27 | 3-hydroxyoctanoyl carnitine | −0.53 | 0.59 |
| V28 | 1-Methylnicotinamide | −0.63 | 0.53 |
| V29 | 3-hydroxydecanoyl carnitine | −0.73 | 0.48 |
| V30 | Hexanoylcarnitine | −0.79 | 0.45 |
| V31 | Decanoylcarnitine | −0.81 | 0.44 |
| V32 | Ecgonine | −0.81 | 0.44 |
| V33 | 2-trans,4-cis-Decadienoylcarnitine | −0.85 | 0.43 |
| V34 | Ethyl 3-oxohexanoate | −1.20 | 0.30 |
| V35 | Lactic acid | −1.62 | 0.20 |
| V36 | Arabinosylhypoxanthine | −1.72 | 0.18 |
| V37 | Lysine | −3.68 | 0.03 |
| | constant | 7.11 | / |

The equation of model I was: In[P/(1−P)]=3.08×V1+1.26×V2+0.7×V3+0.64×V4+0.41×V5+0.4×V6+0.38×V7+0.31×V8+0.31×V9+0.1×V10+0.09×V11+0.09×V12+0.04×V13−0.04×V14−0.04×V15−0.07×V16−0.12×V17−0.17×V18−0.24×V19−0.24×V20−0.26×V21−0.31×V22−0.32×V23−0.44×V24−0.44×V25−0.49×V26−0.53×V27−0.63×V28−0.73×V29−0.79×V30−0.81×V31−0.81×V32−0.85×V33−1.2×V34−1.62×V35−1.72×V36−3.68×V37+7.11.

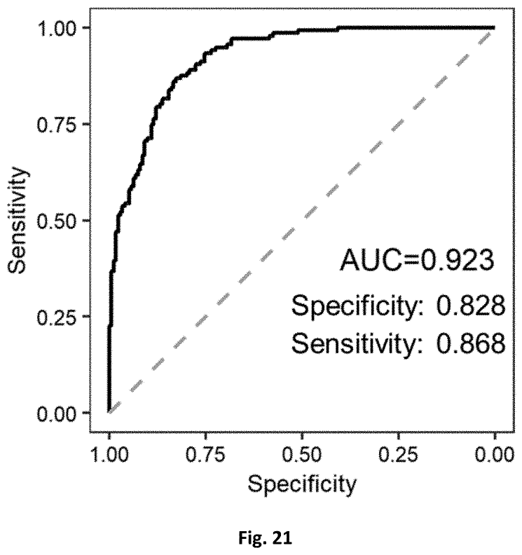

The cut-off value of P was 0.456. That was, the serum detection values (relative abundances) of the aforementioned markers was substituted into the equation of model I for calculation. When P>0.456, it was identified as lung cancer; and when P≤0.456, it was identified as healthy people. ROC analysis was conducted (as shown in FIG. 21), and the model I had a AUC of 0.923, and specificity and sensitivity of 0.828 and 0.868 respectively.

The models A to I described above were just illustrative examples for showing models for auxiliary diagnosis of lung cancer as established by a combination of multiple biomarkers. These models are of great value for the differential diagnosis of lung cancer. It should be noted that the aforementioned models are not exhaustive, and the models established by selecting and combining multiple biomarkers from Table 3 or multiple biomarkers from Table 4 should fall within the scope of the present application, and also had diagnostic values. Meanwhile, it was not limited to the biomarkers in Table 3 or 4, and the biomarkers in Table 3 or 4 could also be selected to establish a model for auxiliary diagnosis of lung cancer together with known biomarkers.

1. A method for detecting whether an individual has lung cancer, comprising: detecting a biomarker in a biosample so as to determine a concentration of the biomarker or relative abundance of the biomarker, wherein the biomarker is selected from one or more of: 1-Methylnicotinamide, 2-Ketobutyric acid, 2-Octenoylcarnitine, 2−Pyrrolidone, 2-trans,4-cis-Decadienoylcarnitine, 3b,16a-Dihydroxyandrostenone sulfate, 3-Chlorotyrosine, 3-hydroxybutyryl carnitine, 3-hydroxydecanoyl carnitine, 3-hydroxydodecanoyl carnitine, 3-hydroxyoctanoyl carnitine, 4-oxo-Retinoic acid, 7-Methylguanine, Acetophenone, Acetylcarnitine, Alanine, alpha-Eleostearic acid, Aminoadipic acid, Arabinosylhypoxanthine, Asparagine, Bilirubin, Carnitine, Choline Sulfate, cis-5-Tetradecenoylcarnitine, Citrulline, Creatinine, Cyclohexaneacetic acid, Diethylamine, Dihydrothymine, Dihydroxybenzoic acid, Docosahexaenoic acid, Ecgonine, Ergothioneine, Ethyl 3-oxohexanoate, Glutamine, Hexanoylcarnitine, Hippuric acid, Homo-L-arginine, Hydroxybutyric acid, Hypoxanthine, Inosine, Isoleucine, Kynurenine, Lactic acid, Leucine, Linoleyl carnitine, Lysine, Methylacetoacetic acid, N6,N6,N6-Trimethylysine, N-Acetyl-L-alanine, Nicotine, Octanoylcarnitine, 5-Oxoproline, Phenylalanine, Pilocarpine, Propionylcarnitine, Pyruvic acid, Serotonin, Succinic acid semialdehyde, Trimethylamine N-oxide, Tyrosine, Uridine, Urocanic acid, Xanthine, 4-Hydroxyphenylacetic acid, Dehydroepiandrosterone sulfate, Androsterone sulfate, Dihydrotestosterone sulfate, Epiandrosterone sulfate, Citric acid, Uric acid, Pantothenic acid, Indole-3-acetic acid, gamma-Butyrobetaine, Calcitriol, all-trans-retinal, 3,4-dihydroxyphenylacetic acid, Caprylic acid, Arachidic acid, Hydrocortisone Valerate, Dopamine, Tryptophan, 3-Hydroxybutyric acid, Arachidonic acid, Decanoylcarnitine, 3-hydroxydodecanoylcarnitine, 3-hydroxydecanoylcarnitine, 3-hydroxyoctanoylcarnitine, Homo-L-arginine, 17-Hydroxypregnenolone sulfate, Pregnenolone sulfate, Tiglylcarnitine, 3-hydroxybutyrylcarnitine, Oxindole, Phenylacetylglutamine, Aminocaproic acid, Methylimidazoleacetic acid.

2. The method according to claim 1, wherein the biomarker is selected from one or more of: alpha-Eleostearic acid, 2-Ketobutyric acid, 2-Octenoylcarnitine, 2-trans, 4-cis-Decadienoylcarnitine, 3-Chlorotyrosine, 3-hydroxydecanoyl carnitine, 3-hydroxydodecanoyl carnitine, 3-hydroxyoctanoyl carnitine, Acetophenone, Arabinosylhypoxanthine, Cyclohexaneacetic acid, Dihydroxybenzoic acid, Docosahexaenoic acid, Ecgonine, Ethyl 3-oxohexanoate, Hexanoylcarnitine, Hippuric acid, Homo-L-arginine, Hypoxanthine, Lactic acid, N-Acetyl-L-alanine, Octanoylcarnitine, 5-Oxoproline, Pyruvic acid, Serotonin, Succinic acid semialdehyde, Xanthine.

3. The method according to claim 1, wherein the biomarker is selected from one or more of: 3-hydroxydodecanoyl carnitine, Arabinosylhypoxanthine, Cyclohexaneacetic acid, Ecgonine, Ethyl 3-oxohexanoate, Hippuric acid, Homo-L-arginine, Hypoxanthine, Octanoylcarnitine, 5-Oxoproline.

4. The method according to claim 1, wherein the detection comprises detecting whether an individual without nodules in the lung has lung cancer.

5. The method according to claim 1, wherein the detection comprises detecting whether an individual with pulmonary nodules has lung cancer.

6. The method according to claim 5, wherein the biomarker is selected from one or more of: 1-Methylnicotinamide, 2-Pyrrolidone, 4-oxo-Retinoic acid, 7-Methylguanine, Acetylcarnitine, Bilirubin, Choline Sulfate, cis-5-Tetradecenoylcarnitine, Citrulline, Creatinine, Diethylamine, Dihydrothymine, Glutamine, Hydroxybutyric acid, Inosine, Kynurenine, Linoleyl carnitine, Lysine, Trimethylamine N-oxide.

7. The method according to claim 5, wherein the biomarker is selected from one or more of: 1-Methylnicotinamide, 2-Octenoylcarnitine, 3-hydroxydecanoyl carnitine, 3-hydroxyoctanoyl carnitine, 4-oxo-Retinoic acid, 7-Methylguanine, Arabinosylhypoxanthine, Cyclohexaneacetic acid, Ecgonine, Ethyl 3-oxohexanoate, Hippuric acid, Homo-L-arginine, Hypoxanthine, Inosine, Lactic acid, Octanoylcarnitine, 5-Oxoproline, Trimethylamine N-oxide.

8. (canceled)

9. (canceled)

10. The methodusc according to claim 19, wherein the biomarker is selected from one or more of: 1-Methylnicotinamide, 2-trans,4-cis-Decadienoylcarnitine, 3-hydroxydecanoyl carnitine, 3-hydroxydodecanoyl carnitine, 3-hydroxyoctanoyl carnitine, 4-oxo-Retinoic acid, 7-Methylguanine, Acetylcarnitine, alpha-Eleostearic acid, Arabinosylhypoxanthine, Cyclohexaneacetic acid, Diethylamine, Docosahexaenoic acid, Ecgonine, Ethyl 3-oxohexanoate, Glutamine, Hippuric acid, Homo-L-arginine, Hypoxanthine, Inosine, Linoleyl carnitine, N-Acetyl-L-alanine, Octanoylcarnitine, 5-Oxoproline, Pyruvic acid, Trimethylamine N-oxide.

11. The method according to claim 19, wherein the biomarker is selected from one or more of: 2-Octenoylcarnitine, 3-hydroxybutyryl carnitine, Aminoadipic acid, Bilirubin, Dihydrothymine, Ergothioneine, Lactic acid, N6,N6,N6-Trimethylysine, Nicotine.

12. The method according to claim 19, wherein the biomarker is selected from one or more of: alpha-Eleostearic acid, 2-Octenoylcarnitine, 2-trans,4-cis-Decadienoylcarnitine, 3 -hydroxydecanoyl carnitine, 3 -hydroxydodecanoyl carnitine, Acetylcarnitine, Bilirubin, Diethylamine, Dihydrothymine, Docosahexaenoic acid, Glutamine, Linoleyl carnitine, N-Acetyl-L-alanine, Pyruvic acid, 3-hydroxybutyryl carnitine, Aminoadipic acid, Ergothioneine, N6,N6,N6-Trimethylysine, Nicotine.

13. The method according to claim 1, wherein the biomarker is selected from one or more of: 3-hydroxybutyryl carnitine, Aminoadipic acid, Ergothioneine, Nicotine.

14. (canceled)

15. (canceled)

16. The method according to claim 1, wherein the biomarker is selected from one or more of: 1-Methylnicotinamide, 2-Ketobutyric acid, 2−Pyrrolidone, 3-Chlorotyrosine, 3-hydroxydecanoyl carnitine, 3-hydroxyoctanoyl carnitine, 4-oxo-Retinoic acid, 7-Methylguanine, Acetophenone, Arabinosylhypoxanthine, Choline Sulfate, Citrulline, Creatinine, Cyclohexaneacetic acid, Ecgonine, Ethyl 3-oxohexanoate, Hexanoylcarnitine, Hippuric acid, Homo-L-arginine, Hypoxanthine, Inosine, Lactic acid, Lysine, Octanoylcarnitine, 5-Oxoproline, Serotonin, Succinic acid semialdehyde, Trimethylamine N-oxide, Xanthine.

17. (canceled)

18. (canceled)

19. The method according to claim 16, wherein the biomarker is Phenylalanine.

20. The method according to claim 1, wherein when it is detected whether an individual with nodules in the lung has lung cancer, the detection method comprises substituting the relative abundance of the biomarker into the following model equation:
Logit(P)=ln[P/((1−P)]=5.553×V04+2.92×V05+2.713×V06−0.332×V07−1.798×V10−7.922×V13−0.593×V14+0.643×V17−2.187×V19−0.992×V20−2.352×V33−1.441×V38+7.214×V39−1.22×V40−1.235×V42+1.61;
wherein V04, V05, V06, V07, V10, V13, V14, V17, V19, V20, V33, V38, V39, V40, V42 are respectively the 5-Oxoproline, N-Acetyl-L-alanine, Hypoxanthine, Cyclohexaneacetic acid, Ethyl 3-oxohexanoate, Arabinosylhypoxanthine, Docosahexaenoic acid, Hydroxybutyric acid, Serotonin, Ecgonine, Lysine, Kynurenine, Inosine, 4-oxo-Retinoic acid, Linoleylcarnitine, and wherein the individual is a woman.

21. method according to claim 1, wherein when it is detected whether an man with nodules in the lung has lung cancer, the detection method comprises substituting the relative abundance of the biomarker into the following model equation:
Logit(P)=ln[P/((1−P)]=6.283×MV02−0.646×MV10−2.758×MV13+1.864×MV15−1.126×MV19−1.145×MV27−3.918×MV30+1.494;
wherein MV02, MV10, MV13, MV15, MV19, MV27, MV30 are respectively the 5-Oxoproline, Nicotine, Ecgonine, N6,N6,N6-Trimethylysine, Arabinosylhypoxanthine, Docosahexaenoic acid, Linoleyl carnitine.

22-43. (canceled)

44. The method according to claim 1, wherein when it is detected whether an individual with nodules in the lung has lung cancer, the relative abundance of the biomarkers is substituted into one or more of the following models:
model A: ln[P/(1−P)]=2.29×M1+1.02×M2+0.64×M3+0.62×M4+0.47×M5+0.42×M6+0.38×M7+0.26×M8+0.05×M9+0.03×M10−0.05×M11−0.12×M12−0.16×M13−0.17×M14−0.36×M15−0.4×M16−0.45×M17−0.46×M18−0.47×M19−0.53×M20−0.55×M21−0.79×M22−0.95×M23−1.02×M24−1.19×M25−1.52 ×M26−1.88×M27+4.01, wherein M1-M27 are respectively relative abundances of Hypoxanthine, Alanine, 2-Ketobutyric acid, 2-trans,4-cis-Decadienoylcarnitine, Xanthine, 17-Hydroxypregnenolone sulfate, Dihydrothymine, Octanoylcarnitine, Lactic acid, Pregnenolone sulfate, 3-Chlorotyrosine, Cyclohexaneacetic acid, Choline Sulfate, Trimethylamine N-oxide, 2-Octenoylcarnitine, 1-Methylnicotinamide, Serotonin, Docosahexaenoic acid, Decanoylcarnitine, alpha-Eleostearic acid, Homo-L-arginine, Pyruvic acid, 3-hydroxydecanoylcarnitine, Ecgonine, Kynurenine, Ethyl 3-oxohexanoate, Arabinosylhypoxanthine;
model B: ln[P/(1−P)]=1.11×M1+0.25×M2+0.13 ×M3+0.09×M4+0.05×M5−0.01×M6−0.02×M7−0.02×M8−0.04×M9−0.11×M10−0.12×M11−0.19×M12−0.3×M13−0.34×M14−0.45×M15−0.46×M16−0.64×M17−0.68×M18−0.95×M19+2.17, wherein M1-M19 are respectively relative abundances of Hypoxanthine, Alanine, 2-Ketobutyric acid, 17-Hydroxypregnenolone sulfate, Dihydrothymine, Trimethylamine N-oxide, Serotonin, 3-Chlorotyrosine, Hippuric acid, Docosahexaenoic acid, 2-Octenoylcarnitine, 1-Methylnicotinamide, Homo-L-arginine, alpha-Eleostearic acid, Kynurenine, 3-hydroxydecanoyl carnitine, Ecgonine, Ethyl 3-oxohexanoate, Arabinosylhypoxanthine;
model C: ln[P/(1−P)]=1.73×M1+0.72×M2+0.31×M3+0.29×M4+0.23×M5+0.15×M6−0.05×M7−0.07×M8−0.07×M9−0.09×M10−0.09×M11−0.16×M12−0.26×M13−0.26×M14−0.27×M15−0.29×M16−0.43×M17−0.45×M18−0.56×M19−0.75×M20−0.87×M21−1.15×M22−1.41×M23+3.07, wherein M1-M23 are respectively relative abundances of Hypoxanthine, Alanine, 2-Ketobutyric acid, 17-Hydroxypregnenolone sulfate, Dihydrothymine, Xanthine, 3-Chlorotyrosine, Cyclohexaneacetic acid, Choline Sulfate, Decanoylcarnitine, Trimethylamine N-oxide, 2-Octenoylcarnitine, PyruMic acid, Docosahexaenoic acid, 1-Methylnicotinamide, Serotonin, Homo-L-arginine, alpha-Eleostearic acid, 3-hydroxydecanoyl carnitine, Ecgonine, Kynurenine, Ethyl 3-oxohexanoate, Arabinosylhypoxanthine;
model D: ln[P/(1−P)]=2.35×M1+1.03×M2+0.71×M3+0.68×M4+0.48×M5+0.45×M6+0.42×M7+0.39×M8+0.16×M9+0.03×M10−0.05×M11−0.12×M12−0.17×M13−0.17×M14−0.22×M15−0.39×M16−0.43×M17−0.46×M18−0.49×M19−0.54×M20−0.54×M21−0.57×M22−0.89×M23−0.97×M24−1.07×M25−1.23×M26−1.56×M27−1.93×M28+4.16, wherein M1-M28 are respectively relative abundances of Hypoxanthine, Alanine, 2-Ketobutyric acid, 2-trans,4-cis-Decadienoylcarnitine, Xanthine, Octanoylcarnitine, 17-Hydroxypregnenolone sulfate, Dihydrothymine, Lactic acid, Pregnenolone sulfate, 3-Chlorotyrosine, Cyclohexaneacetic acid, Choline Sulfate, Trimethylamine N-oxide, Hexanoylcarnitine, 2-Octenoylcarnitine, 1-Methylnicotinamide, Serotonin, Docosahexaenoic acid, Decanoylcarnitine, alpha-Eleostearic acid, Homo-L-arginine, Pyruvic acid, 3-hydroxydecanoyl carnitine, Ecgonine, Kynurenine, Ethyl 3-oxohexanoate, Arabinosylhypoxanthine;
model E: ln[P/(1−P)]=1.41×V1+0.26×V2+0.04×V3−0.01×V4−0.05×V5−0.09×V6−0.19×V7−0.2×V8−0.32×V9−0.34×V10−0.4×V11−0.48×V12−0.55×V13−0.64×V14−1.07×V15−1.58×V16+3.44, V1-V16 are respectively relative abundances of Hypoxanthine, Alanine, 2-Ketobutyric acid, 3-hydroxybutyrylcarnitine, Nicotine, Hippuric acid, Citrulline, Trimethylamine N-oxide, alpha-Eleostearic acid, 1-Methylnicotinamide, 3-hydroxydecanoylcarnitine, Ecgonine, Ethyl 3-oxohexanoate, 2-trans,4-cis-Decadienoylcarnitine, Arabinosylhypoxanthine, Lysine;
model F: ln[P/(1−P)]=1.51×V1+0.29×V2+0.06×V3−0.03×V4−0.03×V5−0.03×V6−0.07×V7−0.1×V8−0.21×V9−0.22×V10−0.33×V11−0.35×V12−0.39×V13−0.51×V14−0.59×V15−0.63 ×V16−1.12×V17−1.69×V18+3.65, wherein V1-V18 are respectively relative abundances of Hypoxanthine, Alanine, 2-Ketobutyric acid, 3-hydroxybutyryl carnitine, Decanoylcarnitine, Ergothioneine, Nicotine, Hippuric acid, Trimethylamine N-oxide, Citrulline, alpha-Eleostearic acid, 1-Methylnicotinamide, 3-hydroxydecanoyl carnitine, Ecgonine, Ethyl 3-oxohexanoate, 2-trans, 4-cis-Decadienoylcarnitine, Arabinosylhypoxanthine, Lysine;
model G: ln[P/(1−P)]=1.67×V1+0.34×V2+0.1×V3+0.01×V4−0.08×V5−0.08×V6−0.09×V7−0.1×V8−0.12×V9−0.23×V10−0.27×V11−0.36×V12−0.36×V13−0.38×V14−0.56×V15−0.61×V16−0.66×V17−1.2×V18−1.89×V19+4, wherein V1-V19 are respectively relative abundances of Hypoxanthine, Alanine, 2-Ketobutyric acid, Aminocaproic acid, 3-hydroxybutyryl carnitine, Ergothioneine, Decanoylcarnitine, Nicotine, Hippuric acid, Trimethylamine N-oxide, Citrulline, 3-hydroxydecanoyl carnitine, alpha-Eleostearic acid, 1-Methylnicotinamide, Ecgonine, 2-trans,4-cis-Decadienoylcarnitine, Ethyl 3-oxohexanoate, Arabinosylhypoxanthine, Lysine;
model H: ln[P/(1−P)]=2.03×V1+0.47×V2+0.15×V3+0.09×V4+0.04×V5+0.03×V6+0.01×V7−0.12×V8−0.12×V9−0.13×V10−0.14×V11−0.14×V12−0.27×V13−0.36×V14−0.37×V15−0.37×V16−0.4×V17−0.43×V18−0.59×V19−0.63×V20−0.75×V21−1.37×V22−2.18×V23+4.56, wherein V1-V22 are respectively relative abundances of Hypoxanthine, Alanine, 2-Ketobutyric acid, Tiglylcarnitine, N6,N6,N6-Trimethylysine, Aminocaproic acid, Oxindole, Decanoylcarnitine, Nicotine, Ergothioneine, 3-hydroxybutyryl carnitine, Hippuric acid, Trimethylamine N-oxide, Lactic acid, Citrulline, alpha-Eleostearic acid, 3-hydroxydecanoyl carnitine, 1-Methylnicotinamide, 2-trans,4-cis-Decadienoylcarnitine, Ecgonine, Ethyl 3-oxohexanoate, Arabinosylhypoxanthine, Lysine; or
model I: ln[P/(1−P)]=3.08×V1+1.26×V2+0.7×V3+0.64×V4+0.41×V5+0.4×V6+0.38×V7+0.31×V8+0.31×V9+0.1×V10+0.09×V11+0.09×V12+0.04×V13−0.04×V14−0.04×V15−0.07×V16−0.12×V17−0.17×V18−0.24×V19−0.24×V20−0.26×V21−0.31×V22−0.32×V23−0.44×V24−0.44×V25−0.49×V26−0.53×V27−0.63×V28−0.73×V29−0.79×V30−0.81×V31−0.81×V32−0.85×V33−1.2×V34−1.62×V35−1.72×V36−3.68×V37+7.11, wherein V1-V37 are respectively relative abundances of Hypoxanthine, Octanoylcarnitine, Alanine, 3-hydroxydodecanoyl carnitine, Xanthine, 2-Ketobutyric acid, Oxindole, Tiglylcarnitine, N6,N6,N6-Trimethylysine, Cyclohexaneacetic acid, Aminocaproic acid, Methylimidazoleacetic acid, Homo-L-arginine, Pyruvic acid, 2-Octenoylcarnitine, Propionylcarnitine, Nicotine, Serotonin, Phenylacetylglutamine, Hippuric acid, Ergothioneine, 3-hydroxybutyryl carnitine, alpha-Eleostearic acid, Inosine, Citrulline, Trimethylamine N-oxide, 3-hydroxyoctanoyl carnitine, 1-Methylnicotinamide, 3-hydroxydecanoyl carnitine, Hexanoylcarnitine, Decanoylcarnitine, Ecgonine, 2-trans,4-cis-Decadienoylcarnitine, Ethyl 3-oxohexanoate, Lactic acid, Arabinosylhypoxanthine, Lysine.

45-50. (canceled)

51. The method according to claim 1, wherein the biomarker is selected from one or more of: Decanoylcarnitine, 17-Hydroxypregnenolone sulfate, Pregnenolone sulfate, Tiglylcarnitine, Oxindole, Phenylacetylglutamine, Aminocaproic acid, Methylimidazoleacetic acid.

52. The method according to claim 1, wherein the biomarker is selected from one or more of: 2-trans,4-cis-Decadienoylcarnitine, Octanoylcarnitine, Decanoylcarnitine, 2-Octenoylcarnitine, Hexanoylcarnitine, 3 -hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine.

53. The method according to claim 1, wherein the biomarker is selected from one or more of: 2-trans,4-cis-Decadienoylcarnitine, Octanoylcarnitine, Decanoylcarnitine, 2-Octenoylcarnitine, Hexanoylcarnitine, 3 -hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine, Kynurenine, cis-5-Tetradecenoylcarnitine, Docosahexaenoic acid, Choline Sulfate, Dihydrothymine, 17-Hydroxypregnenolone sulfate, Pregnenolone sulfate.

54. The method according to claim 1, wherein the biomarker is selected from one or more of: 2-trans,4-cis-Decadienoylcarnitine, Octanoylcarnitine, Decanoylcarnitine, 2-Octenoylcarnitine, Hexanoylcarnitine, 3 -hydroxydodecanoylcarnitine, 3-hydroxydecanoylcarnitine, 3-hydroxyoctanoylcarnitine, Ecgonine, Trimethylamine N-oxide, 1-Methylnicotinamide, 3-Chlorotyrosine, Homo-L-arginine, Serotonin, Alanine, alpha-Eleostearic acid, Ethyl 3-oxohexanoate, Inosine, Arabinosylhypoxanthine, Hippuric acid, Cyclohexaneacetic acid, Lactic acid, 2-Ketobutyric acid, Pyruvic acid, Hypoxanthine, Xanthine, N6,N6,N6-Trimethylysine, Tiglylcarnitine, Propionylcarnitine, 3-hydroxybutyrylcarnitine, Oxindole, Nicotine, Ergothioneine, Phenylacetylglutamine, Citrulline, Lysine, Aminocaproic acid, Methylimidazoleacetic acid.
